(19) United States
(12) Patent Application Publication
(10) Pub. No.: US 2014/0088192 A1
(43) Pub. Date: Mar. 27, 2014

(54) SUBCUTANEOUSLY INFUSIBLE LEVODOPA PRODRUG COMPOSITIONS AND METHODS OF INFUSION

(71) Applicants: Ephraim Heller, Wilson, WY (US); Adam Heller, Austin, TX (US)

(72) Inventors: Ephraim Heller, Wilson, WY (US); Adam Heller, Austin, TX (US)

(73) Assignee: SynAgile Corporation, Wilson, WY (US)

(21) Appl. No.: 13/992,105

(22) PCT Filed: Dec. 12, 2011
(86) PCT No.: PCT/US11/64398

§ 371 Date: Dec. 11, 2013

Related U.S. Application Data
(60) Provisional application No. 61/421,902, filed on Dec. 10, 2010.
(60) Provisional application No. 61/431,256, filed on Jan. 10, 2011.
(60) Provisional application No. 61/492,227, filed on Jun. 1, 2011.
(60) Provisional application No. 61/538,449, filed on Sep. 23, 2011.

(51) Int. Cl. A61K 31/216

(52) U.S. Cl. CPC A61K 31/216
USPC 514/538

(57) ABSTRACT

The invention features methods compositions and infusion pumps for infusing levodopa prodrugs (e.g., levodopa esters, levodopa amides, levodopa carboxamides, and levodopa sulfonamides) for the treatment of Parkinson's disease.

### BACKGROUND OF THE INVENTION

The invention relates to compositions, including levodopa esters, for the treatment of Parkinson's disease.

Parkinson's disease (PD) is characterized by the inability of the dopaminergic neurons in the substantia nigra to produce the neurotransmitter dopamine. PD impairs motor skills, cognitive processes, autonomic functions and sleep. Motor symptoms include tremor, rigidity, slow movement (bradykinesia), and loss of the ability to initiate movement (akinesia) (collectively, the “off” state). Non-motor symptoms of PD include dementia, dysphagia (difficulty swallowing), slurred speech, orthostatic hypotension, seborrheic dermatitis, urinary incontinence, constipation, mood alterations, sexual dysfunction, and sleep issues (e.g., daytime somnolence, insomnia).

After more than 40 years of clinical use levodopa therapy remains the most effective method for managing PD and provides the greatest improvement in motor function. Consequently, levodopa (LD) administration is the primary treatment for PD. Levodopa is usually orally administered. The orally administered levodopa enters the blood and part of the levodopa in the blood crosses the blood brain barrier. It is metabolized, in part, in the brain to dopamine which temporarily diminishes the motor symptoms of PD. As the neurodegeration underlying PD progresses, the patients require increasing doses of levodopa and the fluctuations of brain dopamine levels increase. When too much levodopa is transported to the brain, dyskinesia sets in, (uncontrolled movements such as writhing, twitching and shaking); when too little is transported, the patient re-enters the off state. As PD progresses, the therapeutic window for oral formulations of levodopa narrows, and it becomes increasingly difficult to control PD motor symptoms without inducing motor complications. In addition, most PD patients develop response fluctuations to oral levodopa therapy, such as end of dose wearing off, sudden on/off's, delayed time to on, and response failures.

Besides levodopa, other drugs commonly used for treatment of PD include DDC inhibitors, such as carbidopa and benserazide; dopamine receptor agonists, such as pramipexole, ropinirole, bromocriptine, pergolide, piribedil, cabergoline, lisuride, and apomorphine; MAO-B inhibitors, such as rasagiline and selegiline; COMT inhibitors, such as entacapone and tolcapone; anticholinergics, such as trihexiphenidyl, benztropine, biperiden, and ethopropazine; and amantadine.

Most of the oral levodopa is metabolized before reaching the brain. Peripheral levodopa metabolization to dopamine causes nausea, tremors, and stiffness. Nausea is reduced and bioavailability in the brain is increased by co-administration of DDC-inhibitors, primarily CD or benserazide. CD extends the plasma half-life of levodopa to approximately 90 minutes. These DDC-inhibitors do not substantially cross the blood-brain barrier and thus inhibit only peripheral DDC. The results are reduction in side effects caused by dopamine on the periphery and increase of the concentration of levodopa and dopamine in the brain.

Standard levodopa treatment with oral delivery typically leads to intermittent plasma levodopa levels, which are thought to contribute to motor complications. By contrast, more continuous delivery of levodopa that provides smooth, predictable plasma levels leads to a good therapeutic response with reduced motor complications.

The development of an effective controlled release oral dosage form of levodopa that provides substantially reduced variability in plasma levodopa concentrations and more stable, continuous levodopa delivery to the brain is difficult. Some of the underlying causes of this difficulty, and of the response fluctuations themselves, are believed to be: (a) the short biological half-life of levodopa; (b) erratic gastric emptying, due to effects of PD on the autonomic nervous system; (c) poor absorption of levodopa in the gut in the presence of food, due to competition between levodopa and other amino acids for transport across the intestines; (d) absorption of levodopa taking place only in the duodenum, a short segment of the intestines; and (e) competition between levodopa and other amino acids for active transport from the blood into the brain.

Numerous studies demonstrate that IV infusion of levodopa stabilizes its concentration in plasma and dramatically reduces motor complications and fluctuations (see, for example, Shoulson et al., Neurology 25:1144 (1975); Rosin et al., Arch Neurol. 36:32 (1979); Quinn et al., Lancet. 2:412 (1982); Quinn et al., Neurology. 34:1131 (1984); Nutt et al., N Engl J. Med. 310:483 (1984); Hardie et al., Br J Clin Pharmac. 22:429 (1986); and Hardie et al., Brain. 107:487 (1984)).

Likewise, many studies show similarly favorable results upon continuous levodopa infusion directly into the duodenum, using an ambulatory infusion pump (Duodopa therapy). Studies of Duodopa therapy confirm >50% reductions in time spent in the “off” state and time spent with severe dyskinesias. These studies also demonstrate significant improvement in quality of life of the patients (see, for example, Bredberg et al., Eur J Clin Pharmacol. 45:117 (1993); Kurth et al., Neurology 43:1698 (1993); Nilsson et al., Acta Neurol Scand. 97:175 (1998); Syed et al., Mov Disord. 13:336 (1998); Nilsson et al., Acta Neurol Scand. 104:343 (2001); Nyholm et al., Clin Neuropharmacol. 26:156 (2003); Nyholm et al., Neurology. 65:1506 (2005); and Nyholm et al., Clin Neuropharmacol. 31:63 (2008); Antonini et al., Mov Disord. 22:1145 (2007)).

Chronic subcutaneous infusion of drugs such as insulin and pain medications is widely practiced. Such systems are safe for chronic use by patients outside the hospital, convenient, and relatively low cost. It would be desirable to be able to also deliver levodopa or a levodopa prodrug subcutaneously.

The practicality of subcutaneous levodopa infusion depends on the liquid volume that must be infused for the typical daily dose of 0.3-2 g of levodopa. The subcutaneous infusion of large volumes can cause persistent swelling and edema.

Levodopa is poorly soluble in aqueous solutions near neutral pH. For example, at 25° C. and at pH 5 the solubility of levodopa is only about 2.8 g per liter, and at neutral pH it is even less soluble, only about 1.65 g per liter. A patient requiring 1 g levodopa per day would correspondingly require the daily infusion of 0.36 liters of the pH 5 solution and of 0.6 liters of the neutral pH solution. In early studies of IV levodopa infusion, volumes of over 2 L of solution (saline or dextrose and water) per day with less than 1 mg/mL of levodopa were often administered making this administration very cumbersome. The acidity of the infusion substance can create an increased risk of thrombophlebitis, and to reduce this risk, central venous access was often utilized.

The two most widely tested levodopa prodrugs are its methyl ester, known as Melevodopa or LDME, and its ethyl ester, known as Etilevodopa or LDEE (see, for example, Stocchi et al., Mov Disord 25:1881 (2010); Stocchi et al., Clin Neuropharmacol 33:198 (2010); Djaldetti et al., Clin Neuropharmacol 26:322 (2003); and Blindauer et al., Arch Neurol 63:210 (2006)). LDME and LDEE can be unstable in solution, making them difficult to store.

The invention features stable compositions that can permit subcutaneous administration of levodopa, or a levodopa prodrug, for the treatment of Parkinson's disease.

### ABBREVIATIONS AND DEFINITIONS

The term “CD” refers to Carbidopa.

The term “carbidopa prodrug” refers to carbidopa esters, carbidopa amides, and salts thereof, such as the hydrochloride salt of carbidopa ethyl ester, carbidopa methyl ester, or carbidopa amide.

The term “COMT” refers to catechol-O-methyl transferase.

The term “DDC” refers to DOPA decarboxylase.

The term “hyaluronic acid” refers to hyaluronic acid and salts thereof.

The term “IV” refers to intravenous.

The term “LD” refers to levodopa, also known as L-DOPA, or a salt thereof.

The term “LD₅₀” refers to the median lethal oral dose of an LD prodrug in rats at 48 hours (e.g., the dose of LD prodrug required to kill half the rats within 48 hours after ingestion of the LD prodrug).

The term “LDA” refers to an LD prodrug that is a levodopa amide of formula (III):

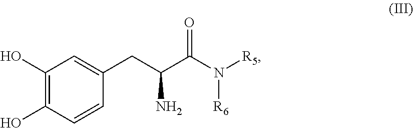

or a pharmaceutically acceptable salt thereof. In formula (III), each of R₅ and R₆ is, independently, selected from H, C₁₋₆ alkyl, C₂₋₆ alkenyl, C₂₋₆ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. In particular preferred embodiments, R₅ is H or CH₃, and R₆ is CH₃, CH₂CH₃, CH₂CH₂CH₃, benzyl, 2-deoxy-2-glucosyl, or CH₂CH₂NH₂. LDAs are hydrolyzed in vivo to form LD and an amine or ammonium salt. The LDAs of the invention and their hydrolysis products have an LD₅₀ in rats of greater than 3 millimoles/kg. The LDA can be administered, for example, in its free base form, or as an acid addition salt.

The term “LDC” refers to an LD prodrug that is a levodopa carboxamide of formula (II):

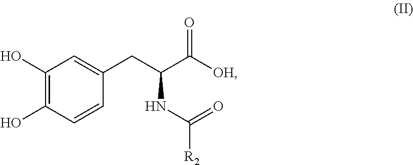

or a pharmaceutically acceptable salt thereof. In formula (II), R₂ is selected from C₁₋₆ alkyl, C₂₋₆ alkenyl, C₂₋₆ alkynyl, C₆₋₁₂ aryl, C₇₋₁₄ alkaryl, C₃₋₁₀ alkheterocyclyl, and C₁₋₇ heteroalkyl. In particular preferred embodiments, R₂ is CH₂CH₃, CH(OH)CH₃, CH₂CH₂COOH, CH₂CH₂CH₃, benzenepropenyl, phenyl, or (CHOH)₄CH₂OH. LDCs are hydrolyzed in vivo to form LD and a carboxylate or carboxylic acid. The LDCs of the invention and their hydrolysis products have an LD₅₀ in rats of greater than 3 millimoles/kg. The LDC can be administered, for example, in its neutral form, or as an alkali metal or alkaline earth salt.

The term “LDE” refers to an LD prodrug that is a levodopa ester of formula (I):

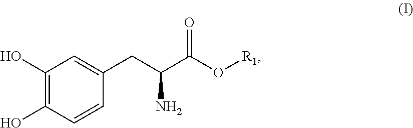

or a pharmaceutically acceptable salt thereof. In formula (I), R₁ is selected from C₁₋₆ alkyl, C₂₋₆ alkenyl, C₂₋₆ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. In particular preferred embodiments, OR₁ is OCH₃, OCH₂CH₃, OCH₂CH₂CH₃, OCH(CH₃)₂, OCH₂CH₂CH₂CH₃, OCH(CH₃)CH₂CH₃, O-benzyl, O-cyclohexyl, OCH₂CH₂OH, OCH₂CH(CH₃)OH, an LD ester of sorbitol, an LD ester of mannitol, an LD ester of xylitol, or an LD ester of glycerol. LDEs are hydrolyzed in vivo to form LD and an alcohol. The LDEs of the invention and their hydrolysis products have an LD₅₀ in rats of greater than 3 millimoles/kg. The LDE can be administered, for example, in its free base form, or as an acid addition salt.

The term “LDS” refers to an LD prodrug that is a levodopa sulfonamide of formula (IV):

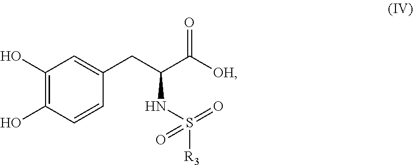

or a pharmaceutically acceptable salt thereof. In formula (IV), R₃ is selected from C₁₋₆ alkyl, C₂₋₆ alkenyl, C₂₋₆ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. In particular preferred embodiments, R₃ is CH₃, or 4-methylbenzyl. LDSs are hydrolyzed in vivo to form LD and a sulfonate. The LDSs of the invention and their hydrolysis products have an LD₅₀ in rats of greater than 3 millimoles/kg. The LDS can be administered, for example, in its neutral form, or as an alkali metal or alkaline earth salt.

The term “LDEE” refers to levodopa ethyl ester, or a salt thereof.

The term “LDME” refers to levodopa methyl ester, or a salt thereof.

The term “LD prodrug” refers to a pharmaceutical composition suitable for infusion, preferably for subcutaneous or intramuscular infusion, forming LD upon its hydrolysis. Examples include LDA, LDE, LDC, LDS, LDEE, and LDME, and their salts. The salts are usually formed, in the cases of LDEs and LDCs, by neutralizing their basic amines with an acid; and, in the cases of LDAs and LDSs, by neutralizing their carboxylic acids or sulfonic acids with a base.

The term “MAO-B” refers to monoamine oxidase-B.

As used herein, “neutral amino acid” refers to an amino acid having only one carboxylic acid and only one amine function. Although phenolic amino acids like LD and OMD are partly ionized to anions and hydrated protons at neutral pH, they are classified as neutral.

The term “PD” refers to Parkinson's disease.

The term “PEG” refers to polyethylene glycol.

As used herein, the term “pH” refers to the pH measured using a pH meter having a glass electrode connected to an electronic meter.

The term “polybasic acid” means an acid having two or more ionizable functions and acid salts of these acids. Examples of polybasic acids include citric acid, succinic acid and phosphoric acid and examples of their acid salts include monosodium citrate, monosodium succinate and monosodium phosphate.

The term “s.c.” refers to subcutaneous.

The term “administration” or “administering” refers to a parenteral method (e.g., infusion, injection, transdermal delivery, or buccal delivery) of giving a dosage of LD or LD prodrug (e.g., LDA, LDE, LDC, or LDS) to a subject. The dosage form of the invention is preferably administered intramuscularly or subcutaneously, optionally using an infusion pump.

As used herein, “aqueous” refers to formulations of the invention including greater than 10% or 20% (w/w) water and, optionally, a cosolvent (e.g., glycerol or ethanol).

As used herein, “coinfused” refers to two or more pharmaceutically active agents, formulated together, or separately, and infused simultaneously, either to the same site (e.g., infused via the same infusion cannula or needle), or adjacent sites (e.g., infused via separate infusion cannulae or needles within 1 cm of each other).

As used herein “continuous administration” or “continuous infusion” refers to both uninterrupted administration/infusion and frequent administration/infusion. In the case of frequent administration/infusion, the frequency is typically at least once per hour, preferably at least twice per hour, more preferably at least four times per hour, and most preferably at least six times per hour. Typical daily durations of continuous administration or infusion typically exceed 12 hours, and are usually 16 hours or 24 hours. The rate of administration or infusion may be reduced during intended sleep periods, optionally to nil.

As used herein, the terms “effective particle size” and “particle size” are used interchangeably and refer to a mixture of particles having a distribution in which 50% of the particles are below and 50% of the particles are above a defined measurement. The “effective particle size” refers to the volume-weighted median diameter as measured by a laser/light scattering method or equivalent, wherein 50% of the particles, by volume, have a smaller diameter, while 50% by volume have a larger diameter. The effective particle size can be measured by conventional particle size measuring techniques well known to those skilled in the art. Such techniques include, for example, sedimentation field flow fractionation, photon correlation spectroscopy, light scattering (e.g., with a Microtrac UPA 150), laser diffraction, and disc centrifugation.

By “fatty acid salt” is meant a fatty acid addition salt of LD or LD prodrug (e.g., LDE, or LDC) in which the anion is a carboxylate, R—C(O)O—, in which R is a saturated or partially-saturated straight chain or branched hydrocarbon group having between 8 and 26 carbon atoms. Fatty acid salts are derived from fatty acids including, without limitation, those occurring naturally in the brain, or found in blood lipids like triglycerides or cholesterol esters. For example, fatty acids having 16 carbon atoms and 0, 1 or 2 double bonds (C16:0; C16:1 and C16:2), those with 18 carbon atoms and 1, 2 or 3 double bonds (C18:1; C18:2; and C18:3), those with 20 carbon atoms and 1, 2 or 4 double bonds (C20:1; C20:2; and C20:4) and those with 22 carbon atoms and 4, 5 or 6 double bonds (C22:4; C22:5 and C22:6). The fatty acids can be substituted or unsubstituted. Exemplary substituents include hydroxyl, halide, methyl, ethyl, propyl, isopropyl, butyl, and pentyl groups. Desirably, the fatty acid salt is 4, 7, 10, 13, 16, 19 docosahexanoate, oleate, ricinoleate, octanoate, alpha-linoleate, eicosapentaenoate, docosahexaenoate, linoleate, gamma linoleate, palmitoleate, dihomogamma linoleate, arachidonate, myristate, palmitate, and stearate.

As used herein, “infused” or “infusion” includes infusion into any part of the body, including the stomach, intestines, abdominal cavity, muscles, fat, dermis, or subcutaneous tissue.

As used herein, “fluid liquid crystal” refers to a liquid dosage form of the invention that includes an ordered phase. The presence of a liquid crystal phase can be identified optically (e.g., via optical properties, such as birefringence).

As used herein, “liquid salt form” refers to a salt of an LD prodrug that is a liquid at 25° C. The liquid salt can be a thermodynamically stable liquid, or it can be a liquid that is thermodynamically metastable and, for example, because of its high viscosity, it does not readily crystallize. When metastable, it is preferred that the liquid salt be stored at about 4° C. or less, where its viscosity is usually higher than it is at 25° C. The liquid is typically clear, although it may contain particles with a particle size smaller than about 1 μm.

As used herein, “liquidus” refers to the temperature of a mixture not having a sharp melting point. The liquidus is the temperature where practically the entire mixture is liquid. While above the liquidus temperature the mixture is usually clear, below the liquidus temperature it usually includes light-scattering crystallites.

As used herein, “Newtonian fluid” refers to a liquid dosage form of the invention that flows regardless of the forces acting on it (e.g., continues to exhibit fluid properties no matter how fast it is stirred or mixed).

As used herein, “non-aqueous” refers to formulations of the invention including less than 10% (w/w) water (e.g., less than 5%, 3%, 2%, 1.5%, 1%, 0.5%, or less than 0.1% (w/w) of the formulation is water).

As used herein, the term “shelf life” means the shelf life of the inventive LD prodrug product sold for use by consumers, during which period the product is suitable for use by a subject. The shelf life of the LD prodrugs of the invention can be greater than 3, 6, 12, 18, or preferably 24 months. The shelf life may be achieved when the product is stored frozen (e.g., at about −18° C.), stored refrigerated (at about 5±3° C., for example at about 4±2° C.), or stored at room temperature (e.g., at about 25° C.). The LD prodrug product sold to consumers may be the solution ready for infusion, or it may be its components. For example, the LD prodrug product for use by consumers may be the dry solid LD prodrug and, optionally, the solution used for its reconstitution; or the LD prodrug stored in an acidic solution and, optionally, a neutralizing basic solution; etc.

As used herein, the term “operational life” means the time period during which the infusion solution containing the LD prodrug is suitable for infusion into a subject, under actual infusion conditions. The operational life of the LD prodrugs of the invention can be greater than 12 hours, 24 hours, 48 hours, 72 hours, 96 hours (4 days), or 7 days. It typically requires that the product is not frozen or refrigerated. The product is often infused at room temperature (e.g., about 25° C.), at body temperature (about 37° C.), or in-between (e.g., 30° C.).

As used herein, “stable” refers to formulations of the invention which are “oxidatively stable” and “hydrolytically stable.” Stable formulations exhibit a reduced susceptibility to chemical transformation (e.g., oxidation and/or hydrolysis) prior to infusion into a subject. Stable dry or liquid formulations are those having a shelf life during which less than 10%, 5%, 4%, 3%, 2% or less than 1% of the LD prodrug (e.g., LDA, LDE, LDC, or LDS) is oxidized or hydrolyzed when stored for a period of 3, 6, 12, 18, or 24 months. In general, the solutions of the stable formulations remain clear, meaning that they have no substantial visible precipitate, after their storage. Stable liquid formulations have an operational life during which less than 10%, 5%, 4%, 3%, 2% or less than 1% of the LD prodrug (e.g., LDA, LDE, LDC, or LDS) is oxidized or hydrolyzed over a period of 8 hours, 12 hours, 16 hours, 24 hours, 48 hours, 72 hours, 96 hours, or 7 days. An “oxidatively stable” formulation exhibits a reduced susceptibility to oxidation during its shelf life and/or its operational life, during which less than 10%, 5%, 4%, 3%, or less than 2% of the LD prodrug (e.g., LDA, LDE, LDC, or LDS) is oxidized. A “hydrolytically stable” formulation exhibits a reduced susceptibility to hydrolysis during its shelf life and/or operational life in which less than 20%, 10%, 5%, 4%, 3%, 2% or less than 1% of the LD prodrug (e.g., LDA, LDE, LDC, or LDS) is hydrolyzed.

As used herein, “substantially free LD precipitate” refers to formulations of the invention that are clear and without visible precipitates of LD.

As used herein, “substantially free of oxygen” refers to compositions of the invention packaged in a container for storage or for use wherein the packaged compositions are largely free of oxygen gas (e.g., less than 10%, or less than 5%, of the gas that is in contact with the composition is oxygen gas) or wherein the partial pressure of the oxygen is less than 15 torr, 10 torr, or 5 torr. This can be accomplished by, for example, replacing a part or all of the ambient air in the container with an inert atmosphere, such as nitrogen, carbon dioxide, argon, or neon, or by packaging the composition in a container under a vacuum.

As used herein, “substantially free of water” refers to compositions of the invention packaged in a container (e.g., a cartridge) for storage or for use wherein the packaged compositions are largely free of water (e.g., less than 2%, 1%, 0.5%, 0.1%, 0.05%, or less than 0.01% (w/w) of the composition is water). This can be accomplished by, for example, drying the constituents of the formulation prior to sealing the container.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to ameliorate the disease and improve the subject's condition. The term “treating” also comprises treating a subject to delay progression of a disease or its symptoms. Thus, in the claims and embodiments, treating is the administration to a subject either for therapeutic or prophylactic purposes.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups.

By “C₁₋₆ alkyl” is meant a branched or unbranched hydrocarbon group having from 1 to 6 carbon atoms. A C₁₋₆ alkyl may be substituted or unsubstituted, may optionally include monocyclic or polycyclic rings. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups.

C₁₋₆ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂₋₆ alkenyl” is meant a branched or unbranched hydrocarbon group containing one or more double bonds and having from 2 to 6 carbon atoms. A C₂₋₆ alkenyl may be substituted or unsubstituted, may optionally include monocyclic or polycyclic rings. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₁₂ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

By “C₂₋₆ alkynyl” is meant a branched or unbranched hydrocarbon group containing one or more triple bonds and having from 2 to 12 carbon atoms. A C₂₋₆ alkynyl may be substituted or unsubstituted, may optionally include monocyclic or polycyclic rings. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₆ alkynyls include, without limitation, ethynyl, 1-propynyl, 2-propynyl, 1-butynyl, 2-butynyl, and 3-butynyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl or heteroalkyl substituted by an aryl group (e.g., benzyl, phenethyl, phenoxyethyl, or 3,4-dichlorophenethyl) having from 7 to 14 carbon atoms.

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, saccharide radicals, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, saccharide radicals.

By “C₃₋₁₀ alkheterocyclyl” is meant an alkyl or heteroalkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

### SUMMARY OF THE INVENTION

The invention features pharmaceutical compositions, devices and methods for the management of PD. Specifically, it features compositions, devices and methods for maintaining plasma LD concentrations in a desired therapeutic range, thereby reducing the motor symptoms, non-motor symptoms, and response fluctuations associated with PD.

This invention features stable aqueous solutions in which the concentration of a levodopa prodrug can be high enough to allow subcutaneous administration of the typical daily dose of about 5 millimoles to Parkinson's disease patients, usually in a volume of less than about 20 mL, preferably less than 15 mL. It also features LD prodrug compositions that can be subcutaneously infused, without nodule formation, and that are sufficiently stable at body temperature to allow their infusion. The compositions are generally solutions of levodopa esters, carboxamides, sulfonamides or amides. These can be rapidly hydrolyzed by enzymes of the body to levodopa and respectively to an alcohol. The invention also features stable aqueous and non-aqueous compositions in which the concentration of a levodopa prodrug can be high enough to allow intragastric, intraduodenal or intrajejunal administration of the typical daily dose of about 5 millimoles to Parkinson's disease patients, usually in a volume of less than about 5 mL, preferably less than 3 mL. They form infusible solutions that can be stable at about 37° C. for at least 16 hours, 1 day or 2 days. The compositions are generally solutions of salts of levodopa esters, carboxamides, sulfonamides or amides. The LD-prodrugs can be rapidly hydrolyzed by enzymes of the body to levodopa and respectively to an alcohol, a carboxylate salt, e.g., a sodium carboxylate; a sulfonate salt, e.g., a sodium sulfonate; or an ammonium salt, e.g., an ammonium chloride.

Advanced PD patients require daily typically about 1±0.5 g, or about 5.0±2.5 millimoles of LD. The LD-prodrug solutions of this invention are concentrated, such that a daily subcutaneously or intramuscularly infused liquid volume can typically be less than 20 mL, less than 15 mL, less than 10 mL or less than 5 mL per infusion site. Optionally, one or more skin-attached patch pumps is/are used for the infusion. The subcutaneously or intramuscularly infused solutions can be stored refrigerated for a period that can be longer than a year. The stored LD-prodrug solutions, typically have a pH of 2.5±0.5. Prior to their subcutaneous or intramuscular infusion, their pH is increased typically to 5.0±0.5, the ready-to-infuse solutions being typically stable at 37° C. for at least 24 hrs. Even more concentrated and therefore smaller daily volumes, typically of less than 5 mL or preferably less than 3 mL, can be intragastrically, intraduodenally or intrajejunally infused in a subject requiring about 1 g or about 5 millimoles of LD per day. The solutions can be hydrolytically and oxidatively stable. They can be stored refrigerated for periods typically longer than a year. The most preferred solutions are those including LDEE salts.

The invention features a method for treating Parkinson's disease in a subject with an infusion of LD prodrug by subcutaneously infusing into the subject an LD prodrug solution at such a rate that: (a) the circulating plasma concentration of the LD prodrug during the infusion does not exceed 100 ng/mL; and (b) a circulating plasma LD concentration greater than 400 ng/mL is continuously maintained for a period of at least 8 hours during the infusion. In certain embodiments, the LD prodrug solution is subcutaneously infused at such a rate that a circulating plasma LD concentration greater than 800 ng/mL, 1,200 ng/mL, or 1,600 ng/mL (e.g., from 300 to 1,200, from 400 to 800, or from 1,000 to 2,000 ng/mL, depending upon the condition of the subject) is continuously maintained for a period of at least 2 hours, 3 hours, 4 hours, or 8 hours during the infusion. In particular embodiments, the LD prodrug solution is subcutaneously infused at such a rate that a circulating plasma LD concentration greater than 400 ng/mL, 800 ng/mL, 1,200 ng/mL, or 1,600 ng/mL (e.g., from 300 to 1,200, from 400 to 800, or from 1,000 to 2,000 ng/mL, depending upon the condition of the subject) is achieved within 60 minutes of the initiation of the infusion. The LD prodrug solution can be subcutaneously infused at such a rate that the circulating plasma concentration of the LD prodrug during the infusion does not exceed 50 ng/mL, 30 ng/mL, or 10 ng/mL. The LD prodrug solution can be subcutaneously infused at such a rate that a circulating plasma LD concentration less than 7,500 ng/mL, 5,000 ng/mL, 2,500 ng/mL, or 2,000 ng/mL is continuously maintained for a period of at least 8 hours during the infusion. In particular embodiments, the subject receives an average daily dose of less than 20 mL, 18 mL, 16 mL, 14 mL, 12 mL, 10 mL, 9 mL, 8 mL, 7 mL, 6 mL, or 5 mL of the infusible LD prodrug solution. The LD prodrug solution can be subcutaneously infused at such a rate that the circulating LD plasma concentration varies by less than +/−20%, +/−15%, or +/−10% from its mean for a period of at least 1 hour, 2 hours, 3 hours, or 4 hours. The method can further include the administration of an effective amount of carbidopa or carbidopa prodrug (e.g, administered orally, transcutaneously by a skin-adhered dermal patch, or by infusion). Carbidopa can be administered, e.g., by subcutaneous co-infusion, as a solution of one of its highly water soluble prodrug salts, exemplified by carbidopa ethyl ester hydrochloride, by carbidopa methyl ester hydrochloride or by carbidopa amide hydrochloride. The molar amount of the co-administered carbidopa prodrug can be between one-tenth and one-half of the molar amount of LD, preferably about ¼±⅛th of the molar amount of LD. Preparations of the carbidopa prodrugs, recognized to be L-DOPA decarboxylase inhibitors, are described, for example, in U.S. Pat. Nos. 3,895,052 and 7,101,912, and Patent Publication Nos. DE2062285A and FR2052983A1. In one particular embodiment, the LD prodrug solution includes a greater than 0.3M LD prodrug (e.g., 0.4±0.1, 0.5±0.1, 0.6±0.1, 0.7±0.1, 0.8±0.2, 1.0±0.3, 1.5±0.5, 2.0±0.5, 0.6±0.3, 0.75±0.25, 1.0±0.5, 1.5±0.5, 2.0±0.5, 2.5±0.5, 3.0±0.5, 3.5±0.5, or greater than 3.5 moles per liter moles per liter) and is substantially free of precipitated solid LD when stored for 24 hours at about 25° C. The LD prodrug can be selected from LDAs, LDEs, LDCs, LDSs, and salts thereof. In one particular embodiment, the LD prodrug is LDEE, LDME, or a salt thereof. In particular embodiments, the LD prodrug and the carbidopa or a carbidopa prodrug are coinfused as separate solutions, or are contained in a single solution and infused into the subject. The co-infused, or orally administered, carbidopa or a carbidopa prodrug can be administered in a systemically sub-therapeutic amount (e.g., in an amount sufficient to reduce swelling, inflammation, erythema, or nodule formation at the site of administration) or, optionally, administered in a systemically therapeutic amount in order to reduce the systemic L-DOPA decarboxylase activity on the blood side of the blood brain barrier, e.g. in the kidneys, liver and red blood cells, so as to inhibit the decarboxylase and to increase thereby the half-life of L-DOPA.

The daily infused molar amount of the LD prodrug dose can be less than 0.8, 1.0, 1.2, 1.4, 1.6, 1.8, 2.0 or 2.2 times of the orally taken molar amount of LD.

The infused LD prodrug solution can have a pH of from 4.0 to 6.0 (e.g., 4.2 to 5.0, 4.5±0.3, 4.4±0.2, 4.5±0.5 or 5.0±0.5) and includes from 0.3 M to 4.0 M LDEE (e.g., 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M LDEE). In particular embodiments, the LD prodrug solution includes a buffer, such as citrate, succinate, pyrophosphate, or phosphate buffer. The LD prodrug solution can be subcutaneously infused into the subject via one or more ambulatory infusion pumps. In particular embodiments, the infusion is via two or more infusion pumps. In still other embodiments, the infusion is via a two-compartment infusion pump. In certain embodiments, the method further includes the steps of: (i) providing a solution including greater than 0.3M LD prodrug (e.g., 0.4±0.1, 0.5±0.1, 0.6±0.1, 0.7±0.1, 0.8±0.2, 1.0±0.3, 1.5±0.5, 2.0±0.5, 0.6±0.3, 0.75±0.25, 1.0±0.5, 1.5±0.5, 2.0±0.5, 2.5±0.5, 3.0±0.5, 3.5±0.5, or greater than 3.5 moles per liter moles per liter) and having a pH of 2.3±0.7 (e.g., 1.8±0.3, 2.1±0.3, 2.5±0.3, or 2.7±0.3), wherein less than 3% of the LD prodrug is hydrolyzed when stored at 5±3° C. (e.g., at about 4° C.) for a period of 3 months or longer; (ii) raising the pH of the solution to 4.5±1.0 (e.g., 4.5±0.3, 5.0±0.5, 4.4±0.2, 4.5±0.5, or 4.0±0.5), adjusted, for example, with a salt of citric acid; pyrophosphoric acid, succinic acid, or phosphoric acid, to form the LD prodrug solution while optionally also diluting the solution with a volume of water of less than the volume of the stored LD solution; or of less than twice the volume of the stored LD solution; or of less then three times the volume of the stored LD solution; or of less than four times the volume of the stored LD solution; and (iii) infusing at least a portion of the LD prodrug solution into the subject. Step (iii) is optionally performed within 72 hours, 48 hours, or 24 hours of performing step (ii). The method can alleviate a motor or non-motor complication in a subject afflicted with Parkinson's disease, such as tremor, akinesia, bradykinesia, dyskinesia, dystonia, cognitive impairment, and disordered sleep. The LD prodrug solution is optionally coinfused with hyaluronidase and/or an analgesic (e.g., salicylic acid, or a salt thereof; indomethacin; ibuprofen; amiloride; diclofenac; or calcium salts), or, optionally, an analgesic is topically administered to the subject at the site of injection. The LD prodrug solution can be infused proximate a large muscle (e.g., the diaphragm, trapezius, deltoid, pectoralis major, triceps brachii, biceps, gluteus maximus, sartorius, biceps femoris, rectus femoris, and gastrocnemius) at a depth between 4 mm and 15 mm below the epidermis of the subject.

The invention features a method of treating a patient with Parkinson's disease, including subcutaneously infusing into the patient an aqueous solution including a LD prodrug (such as LDEE) at one or more infusion sites (e.g., one, two, three, four, or more infusion sites), wherein the volume infused at a single infusion site is less than 20 mL (e.g., between 5-20 mL, or 7-12 mL) per 24 hour period; the amount of drug delivered at all infusion sites is less than 10 millimoles (e.g., between 0.25-10 millimoles, or 0.4-0.6 millimoles) per 24 hour period; and the pH of the aqueous solution is between 4.0-6.0 (e.g., 4.0-5.3). For example, 1-10, 1-5, 1-3, 1-2, or 2-3.5 millimoles of LD prodrug can be infused at a single infusion site during a single infusion. It has been empirically determined that infusing LDEE under these conditions reduces the incidence of pain, inflammation, swelling, and subcutaneous nodule formation, while providing adequate operational stability.

The invention also features a kit including: (i) a first container including a sterile aqueous solution, and (ii) a second container including a sterile, dry, reconstitutable solid, wherein either the first container or the second container includes LDEE or a salt thereof. The kit further includes (iii) instructions for combining the contents of the first container with the contents of the second container to form a solution suitable for subcutaneous infusion into a subject and for infusing said solution into a subject for the treatment of Parkinson's disease; wherein said solid fully dissolves in said solution in less than 5 minutes at 25° C.; said infusible solution includes LDEE, or a salt thereof, and has a pH of from 4.0 to 6.0; wherein less than 3% of the LDEE is hydrolyzed when said first container and said second container are stored at 5±3° C. for a period of 3 months. Optionally, subsequent to storage of said first container and the second container at 5±3° C. for a period of 3 months and then forming the infusible solution, the infusible solution remains substantially free of precipitated LD when kept at about 37° C. for at least 24 hours. The pH of the infusible solution may be from about pH 4.0 to pH 5.0, or from pH 5.0 to pH 5.5.

In one embodiment, the sterile, dry, reconstitutable solid of the kit may include LDEE. In another embodiment, the aqueous solution of the kit, stored in a first container that is substantially impermeable to oxygen and under an atmosphere substantially free of oxygen, can include 0.3 M to 4.0 M LDEE or LDEE.HCl and have a pH from 1.0 to 3.5; the second container of the kit can include sterile a solid base.

This invention also features a method of forming, in 5 minutes or less at about 25° C., an infusible, preferably subcutaneously infusible, solution by mixing solid LDE or LDA stored in a first container with an aqueous solution of HCl of a concentration of less than 2 M, 1.5 M, 1M, 0.75M, 0.6 M or 0.5 M, the HCl solution stored in a second container, the HCl solution also including a polybasic acid at a concentration of less than about 1/10^{th} the concentration of the HCl; such that the pH of the infusible solution formed upon mixing all or part of the contents of the two containers is 5.5±0.5, 5.0±0.5 or 4.5±0.5, the solution remaining clear, i.e., precipitate-free, when kept at about 25° C. for 48 hours or longer, or at 37° C. for 16, 24 or 48 hours or longer. Exemplary LDEs include LDEE and LDME. Exemplary polybasic acids include citric acid or phosphoric acid and their acid salts.

This invention also features a kit including solid LDE or LDA in one container; and a second container including aqueous HCl of a concentration less than 2 M, 1.5 M, 1 M, 0.75 M, 0.6 M or 0.5 M HCl and additionally including a polybasic acid at a concentration of less than 1/10^{th} the concentration of the HCl; such that the pH of the infusible, preferably subcutaneously infusible, solution formed upon mixing in 5 minutes or less at about 25° C. part or all the contents of the two containers produces a solution of pH 5.5±0.5, 5.0±0.5 or 4.5±0.5, which remains clear, i.e., precipitate-free, when kept at about 25° C. for more than 48 hours or longer, or at 37° C. for more than 16, 24 or 48 hours, the kit also including instructions for mixing the components. Exemplary LDEs include LDEE and LDME. Exemplary polybasic acids include citric acid and phosphoric acid and their acid salts.

This invention further features a method of forming an infusible, preferably subcutaneously infusible, solution by dissolving in 5 minutes or less at about 25° C. solid LDE or LDA and a solid salt of a polybasic acid of an at least tenfold lesser molar amount than the molar amount of the LDE or the LDA stored in a first container; by adding to the solid mixture HCl of a concentration of less than 2 M, 1.5 M, 1M, 0.75M, 0.6 M or 0.5 M stored in a second container, such that the pH of the resulting solution is 5.5±0.5, 5.0±0.5 or 4.5±0.5, and the solution remains clear, i.e., precipitate-free, when kept at about 25° C. for more than 48 hours or longer or at 37° C. for more than 16 hours. Exemplary LDEs include LDEE and LDME. Exemplary polybasic acid salts include trisodium citrate, disodium citrate, trisodium phosphate or disodium phosphate.

This invention additionally features a kit including solid LDE or LDA and a solid salt of a polybasic acid of an at least tenfold lesser molar amount than the molar amount of the LDE or the LDA in a first container; and HCl of a less than 2 M, 1.5 M, 1M, 0.75M, 0.6 M or 0.5 M concentration in a second container; such that mixing in 5 minutes or less at about 25° C. part or all of the contents of the two containers results in an infusible, preferably subcutaneously infusible, solution of pH 5.5±0.5, 5.0±0.5 or 4.5±0.5, the solution remaining clear, i.e., precipitate-free, when kept at about 25° C. for more than 48 hours or longer or at 37° C. for more than 16 hours, the kit also including instructions for mixing part or all the contents of the two containers and for infusing the solution. Exemplary LDEs include LDEE and LDME. Exemplary polybasic acid salts include trisodium citrate, disodium citrate, trisodium phosphate or disodium phosphate.

The invention features a composition including: (i) a first container including a sterile aqueous solution containing about 0.3M to 4.0M (e.g., 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M) LDEE hydrochloride salt and having a pH of from 1.0 to 3.5 (e.g., 2.3±0.7, 1.8±0.3, 2.1±0.3, 2.5±0.3, or 2.7±0.3), wherein less than 3% of the LDEE is hydrolyzed when the first container is stored at 5±3° C. (e.g., about 4° C.) for a period of 3 months; and (ii) a second container including a sterile basic compound (e.g., trisodium citrate, or any other base described herein) either dissolved in solution or as a solid, reconstitutable base, wherein the combined contents of the first container and the second container form a solution suitable for subcutaneous infusion into a subject, having a pH of from 4.0 to 6.0 (e.g., 4.2 to 5.0, 4.5±0.3, 4.4±0.2, 4.5±0.5 or 5.0±0.5), including greater than or equal to about 0.3 M LDEE (e.g., 0.4±0.1, 0.5±0.1, 0.6±0.3, 1.0±0.3, 1.5±0.5, 2±0.5, or 2.5±0.5 M LDEE), and substantially free of LD precipitate. In particular embodiments, the first container remains substantially free of precipitated LD solids for at least 12 months when stored at about 5±3° C. (e.g., about 4° C.). In still other embodiments, the solution suitable for subcutaneous infusion remains substantially free of precipitated solid LD for at least 48 hours when stored at about 25° C. In yet other embodiments, the solution suitable for subcutaneous infusion remains substantially free of precipitated solid LD for at least 8 hours, e.g., for 16 hours, or for 24 hours or for 48 hours when stored at about 37° C. In particular embodiments, the solution suitable for subcutaneous infusion remains substantially free of precipitated solid LD when thawed after being stored frozen (e.g., at about −18° C. or at about −3° C.) for at least 3 months, 6 months, 12 months, 18 months, or 24 months.

In a related aspect, the invention features a method for treating Parkinson's disease in a subject by (i) providing a first container including a sterile aqueous solution containing about 0.3 M M to 4.0M LDEE hydrochloride salt (e.g., 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M LDEE hydrochloride salt) and having a pH of from 1.0 to 3.5 (e.g., 2.3±0.7, 1.8±0.3, 2.1±0.3, 2.5±0.3, or 2.7±0.3), wherein less than 3% of the LDEE is hydrolyzed when the first container is stored at 5±3° C. (e.g., about 4° C.) for a period of 3 months; (ii) providing a second container including a sterile basic compound (e.g., sodium citrate, or any other base described herein) either dissolved in solution or as a solid, reconstitutable base; (iii) combining the contents of the first container and the second container form a solution suitable for subcutaneous infusion into a subject, having a pH of from 4.0 to 6.0 (e.g., 4.2 to 5.0, 4.5±0.3, 4.4±0.2, 4.5±0.5, or 5.0±0.5), including greater than or equal to about 0.3 M LDEE (e.g., 0.4±0.1, 0.5±0.1, 0.6±0.1, 0.7±0.1, 0.8±0.2, 1.0±0.3, 1.5±0.5, 2±0.5, 1.5±0.5, 2±0.5, or 2.5±0.5 M LDEE), and substantially free of LD precipitate; and (iv) subcutaneously infusing into the subject the solution suitable for subcutaneous infusion.

The invention also features a kit including: (i) a first container including a sterile aqueous solution containing about 0.3 M M to 4.0M LDEE hydrochloride salt (e.g., 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M LDEE hydrochloride salt) and having a pH of from 1.0 to 3.5 (e.g., 2.3±0.7, 1.8±0.3, 2.1±0.3, 2.5±0.3, or 2.7±0.3), wherein less than 3% of the LDEE is hydrolyzed when the first container is stored at 5±3° C. (e.g., about 4° C.) for a period of 3 months; (ii) a second container including sterile basic compound (e.g., sodium citrate, or any other base described herein) either dissolved in solution or as a solid, reconstitutable base; and (iii) instructions for combining the contents of the first container with the contents of the second container to form a solution suitable for subcutaneous infusion into a subject and infusing the solution into a subject for the treatment of Parkinson's disease.

The invention features a container substantially impermeable to oxygen, the container including an atmosphere substantially free of oxygen and including a sterile aqueous solution containing about 0.3 M to 4.0M LDEE hydrochloride salt (e.g., 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M LDEE hydrochloride salt) and having a pH of from 1.0 to 3.5 (e.g., 2.3±0.7, 1.8±0.3, 2.1±0.3, 2.5±0.3, or 2.7±0.3), wherein less than 3% of the LDEE is hydrolyzed when the first container is stored at 5±3° C. (e.g., about 4° C.) for a period of 3 months.

Instead of forming the infused LD prodrug solution by mixing a more acidic aqueous solution and a basic solution, a solution of about pH 4.0±0.5 could be both stored and infused. The LD prodrug solution could be buffered at pH 4.0±0.5 LD, for example with citrate. It could be, for example, an LDE solution, such as an LDEE solution, having a concentration of at least 0.5 M, 1 M, 1.5 M, 2 M, 2.5 M, 3 M. It could be stored refrigerated at 5±3° C., for example at about 4±2° C., for at least 3 months and it could also be infused for 16 hours or longer at ambient temperature, for example at 25±3° C., or even at body temperature, near about 37° C.

The invention features a pharmaceutical composition including an aqueous liquid containing greater than 0.3 M (e.g., 0.3 to 0.6, 0.6 to 1.4, 1.4 to 2.5, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, wherein less than 3% of the LD prodrug is hydrolyzed when the pharmaceutical composition is stored at 5±3° C. (e.g., about 4° C.) for a period of 3 months. In certain embodiments, the aqueous liquid has a pH of from 1.0 to 3.5 (e.g., 2.3±0.7, 1.8±0.3, 2.1±0.3, 2.5±0.3, or 2.7±0.3). The pharmaceutical composition can further include a pharmaceutically acceptable excipient, such as a crystal growth inhibitor, hyaluronic acid, and/or antioxidants. In particular embodiments, the LD prodrug is a hydrochloride salt. In still other embodiments, the liquid has a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 200 cP, or 200 cP to 2,000 cP). The pharmaceutical composition can be substantially free of oxygen. In particular embodiments, the liquid includes a polycarboxylate (e.g., hyaluronic acid, succinylated gelatin, poly(acrylic acid), poly(methacrylic acid), poly(glutamic acid), poly(aspartic acid), poly(maleic acid), poly(malic acid), or poly(fumaric acid)). In still other embodiments, the LD prodrug is an acid addition salt of hydrochloric acid, sulfuric acid, or phosphoric acid. In certain embodiments the pharmaceutical composition is a liquid that is supersaturated in LD. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C. In particular embodiments, the solubility of LD in the pharmaceutical composition is at least 5 g, 10 g, or 15 g per liter at about 25° C. In a related aspect, the invention features a method for treating Parkinson's disease in a subject with an infusion of LD prodrug by (i) providing a pharmaceutical composition described above; (ii) raising the pH of the pharmaceutical composition to 4.0 to 6.0 (e.g., 4.2 to 5.0, 4.5±0.3, 4.4±0.2, 4.5±0.5 or 5.0±0.5) to form an LD prodrug solution; and (iii) within 48 hours, 24 hours, or 12 hours of performing step (ii), infusing at least a portion of the LD prodrug solution into the subject in an amount sufficient to treat Parkinson's disease. In another related aspect, the invention features a method for preparing an infusible LD prodrug solution including the steps of: (i) providing an aqueous liquid containing greater than 0.3 M (e.g., 0.3 to 0.6, 0.6 to 1.4, 1.4 to 2.5, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug and a pH of from 1.0 to 3.5 (e.g., 2.3±0.7, 1.8±0.3, 2.1±0.3, 2.5±0.3, or 2.7±0.3), or a salt thereof, wherein less than 3% of the LD prodrug is hydrolyzed when said pharmaceutical composition is stored at 5±3° C. for a period of 3 months; (ii) raising the pH of the an aqueous liquid to 4.0-6.0 (e.g., 4.2 to 5.0, 4.5±0.3, 4.4±0.2, 4.5±0.5 or 5.0±0.5) to form an infusible LD prodrug solution by combining the aqueous liquid with a base in either reconstitutable solid dosage form or in component solution form (e.g., a base including sodium citrate, or any other base described herein); and (iii) inserting the infusible LD prodrug solution into an infusion pump, wherein the infusible LD prodrug solution remains substantially free of precipitated LD when kept at about 25° C. for at least 24 hours. In one embodiment, the aqueous liquid includes a pharmaceutical composition described above. In still another aspect, the invention features a composition including: (i) a first container including a sterile aqueous solution containing about 0.3 M to 4.0M (e.g., 0.3 to 0.6 M, 0.6 to 1.4, 1.4 to 2.5, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug and having a pH of from 1.0 to 3.5 (e.g., 2.3±0.7, 1.8±0.3, 2.1±0.3, 2.5±0.3, or 2.7±0.3), wherein less than 3% of the LD prodrug hydrolyzed when the first container is stored at 5±3° C. for a period of 3 months; and (ii) a second container including a sterile base either dissolved in solution or as a solid, reconstitutable base (e.g., a base including sodium citrate, or any other base described herein), wherein the combined contents of the first container and the second container form a solution suitable for subcutaneous infusion into a subject, having a pH of from 4.0 to 6.0 (e.g., 4.2 to 5.0, 4.5±0.3, 4.4±0.2, 4.5±0.5, or 5.0±0.5), including greater than or equal to about 0.3 M, e.g., greater than 0.3, 0.4, 0.5, 0.6, 1.0, or 1.5 M LD prodrug, and substantially free of LD precipitate. In one embodiment, the first container includes a pharmaceutical composition described above.

The invention features a pharmaceutical composition suitable for infusion into a subject including an aqueous liquid containing greater than 0.3 M (e.g., 0.3 to 0.6, 0.6 to 1.4 to 2.5, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, wherein the pharmaceutical composition remains substantially free of LD precipitate for at least 24 hours, e.g., for 48 hours or for 72 hours, when stored at about 25° C., or for at least 8 hours, e.g., for 16 hours or 24 hours or 48 hours when stored at about 37° C. In particular embodiments, the aqueous liquid has a pH of from 4.0 to 6.0 (e.g., 4.2 to 5.0, 4.5±0.3, 4.4±0.2, 4.5±0.5, or 5.0±0.5). The pharmaceutical composition can further include a pharmaceutically acceptable excipient, such as a crystal growth inhibitor, hyaluronic acid, and/or antioxidants. In particular embodiments, the LD prodrug is a hydrochloride salt. In still other embodiments, the liquid has a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 200 cP, or 200 cP to 2,000 cP). The pharmaceutical composition can be substantially free of oxygen. In particular embodiments, the liquid includes a polycarboxylate (e.g., hyaluronic acid, succinylated gelatin, poly(acrylic acid), poly(methacrylic acid), poly(glutamic acid), poly(aspartic acid), poly(maleic acid), poly(malic acid), or poly(fumaric acid)). In still other embodiments, the LD prodrug is an acid addition salt of hydrochloric acid, sulfuric acid, or phosphoric acid. In certain embodiments the pharmaceutical composition is a liquid that is supersaturated in LD. In certain embodiments the pharmaceutical composition can remain substantially free of LD precipitate for at least 12 hours, 24 hours, 48 hours, or 72 hours when stored at about 25° C. In some embodiments the pharmaceutical composition can remain substantially free of LD precipitate for at least 8 hours, 16 hours, for example for 24 hours or for 48 hours, when stored at about 37° C. In particular embodiments, the pharmaceutical composition can be substantially free of precipitated solid LD when thawed after being stored frozen (e.g., at about −18° C. or at about −3° C.) for at least 3 months, 6 months, 12 months, 18 months, or 24 months. In still other particular embodiments the solubility of LD in the pharmaceutical composition is at least 5 g, 10 g, or 15 g per liter at about 25° C. In a related aspect, the invention features a method for treating Parkinson's disease in a subject by infusing into the subject a pharmaceutical composition described above in an amount sufficient to treat Parkinson's disease.

The invention features a pharmaceutical composition suitable for infusion into a subject including an aqueous liquid containing greater than 50 weight % of an LD prodrug salt, and containing less than about 40 weight % of water, buffered at a pH between pH 4.0 and pH 5.0, remaining essentially free of LD precipitate after being stored at 5±3° C. (for example at 4±2° C.) for at least 3 months (for example for at least 4 months or 6 months) and/or for at least 8, 16, 24 or 48 hours at about 37° C. An example of such a composition is a buffered, optionally citrate buffered, 2.7 M or greater concentration LDEE.HCl aqueous solution.

The invention features a stable pharmaceutical composition suitable for infusion into a subject, optionally in the jejunum of a subject including greater than 0.3 M (e.g., 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, dissolved in a non-aqueous liquid, wherein the pharmaceutical composition remains substantially free of LD precipitate for at least 24 hours when stored at about 25° C. The non-aqueous liquid can be a lipid (e.g., a triglyceride, a cholesterol ester, sesame oil, castor oil, or cottonseed oil), an alcohol (e.g., ethanol, glycerol or propylene glycol), N-methylpyrrolidone, or a mixture thereof. The aqueous solution can be, e.g., a solution of glucose, glycerol, poly(ethylene glycol), the weight % of the exemplary glucose or glycerol or poly(ethylene glycol) being greater than 10%, 20%, 30%, 40%, 50%. The pharmaceutical composition can include an antioxidant (e.g., bisulfite, propofol, ibuprofen, salicylic acid or salicylic acid salt, a salt of ascorbic acid (such as sodium ascorbate), p-aminophenol, acetamol, a t-butyl ortho-substituted phenol, or any antioxidant described herein). In one embodiment, the pharmaceutical composition can include a fatty acid salt of the LD prodrug. In certain embodiments, the liquid has at about 20° C. a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 100 cP, 50 cP to 500 cP, 250 cP to 750 cP, 500 cP to 1,000 cP, 750 cP to 2,000 cP, or 50 cP to 1,500 cP). In certain embodiments the pharmaceutical composition can remain substantially free of LD precipitate for at least 12 hours, 24 hours, 48 hours, or 72 hours when stored at about 25° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In still other particular embodiments the solubility of LD in the pharmaceutical composition is at least 5 g, 10 g, or 15 g per liter at about 25° C. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C. In a related aspect, the invention features a method for treating Parkinson's disease in a subject by infusing into the subject a pharmaceutical composition described above in an amount sufficient to treat Parkinson's disease.

The invention features a stable pharmaceutical composition suitable for infusion into a subject including greater than 0.3 M (e.g., 0.5±0.2, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, dissolved in a liquid carrier including water and a lipid, wherein the pharmaceutical composition remains substantially free of LD precipitate for at least 24 hours when stored at about 25° C. In particular embodiments, the liquid carrier includes an emulsion or liposomes. The pharmaceutical composition can include an antioxidant (e.g., bisulfite, propofol, ibuprofen, salicylic acid or salicylic acid salt, a salt of ascorbic acid (such as sodium ascorbate), p-aminophenol, acetamol, a t-butyl ortho-substituted phenol, or any antioxidant described herein). In one embodiment, the pharmaceutical composition can include a fatty acid salt of the LD prodrug. In certain embodiments, the pharmaceutical composition has at about 20° C. a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 100 cP, 50 cP to 500 cP, 250 cP to 750 cP, 500 cP to 1,000 cP, 750 cP to 2,000 cP, or 50 cP to 1,500 cP). In certain embodiments the pharmaceutical composition can remain substantially free of LD precipitate for at least 12 hours, 24 hours, 48 hours, or 72 hours when stored at about 25° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In still other particular embodiments the solubility of LD in the pharmaceutical composition is at least 5 g, 10 g, or 15 g per liter at about 25° C. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C. In a related aspect, the invention features a method for treating Parkinson's disease in a subject by infusing into the subject a pharmaceutical composition described above in an amount sufficient to treat Parkinson's disease.

The invention features a pharmaceutical composition including a liquid salt of levodopa methyl ester. In particular embodiments, the liquid salt is a fatty acid salt, such as, without limitation, levodopa methyl ester oleate, levodopa methyl ester octanoate, levodopa methyl ester alpha-linoleate, levodopa methyl ester eicosapentaenoate, levodopa methyl ester docosahexaenoate, levodopa methyl ester linoleate, levodopa methyl ester gamma linoleate, levodopa methyl ester palmitoleate, levodopa methyl ester dihomogamma linoleate, levodopa methyl ester arachidonate, levodopa methyl ester myristate, levodopa methyl ester palmitate, and levodopa methyl ester stearate.

The invention features a pharmaceutical composition including a liquid salt of an LDC. In particular embodiments, the liquid salt is a fatty acid salt, such as, without limitation, a fatty acid salt selected from oleate, octanoate, alpha-linoleate, eicosapentaenoate, docosahexaenoate, linoleate, palmitoleate, gamma linoleate, dihomogamma linoleate, arachidonate, myristate, palmitate, and stearate salts, and mixtures thereof.

The invention features a pharmaceutical composition including a liquid salt of an LDE, wherein the salt is not levodopa ethyl ester oleate, levodopa ethyl ester ricinoleate, levodopa ethyl ester palmitate, or levodopa ethyl ester valerate. In particular embodiments, the liquid salt is a fatty acid salt, such as, without limitation, a fatty acid salt selected from oleate, octanoate, alpha-linoleate, eicosapentaenoate, docosahexaenoate, linoleate, palmitoleate, gamma linoleate, dihomogamma linoleate, arachidonate, myristate, palmitate, and stearate salts, and mixtures thereof.

The invention features a stable pharmaceutical composition suitable for infusion into a subject, optionally into the stomach or duodenum or jejunum of a subject, including greater than 0.3 M (e.g., 0.6±0.3, 0.75±0.25, 1.0±0.5, 1.5±0.5, 2.0±0.5, 2.5±0.5, 3.0±0.5, 3.5±0.5, or greater than 3.5 moles per liter) LD prodrug fatty acid salt, wherein the pharmaceutical composition is substantially free of LD precipitate for at least 12 hours, 24 hours, or 48 hours when stored at about 25° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In particular embodiments, the pharmaceutical composition includes greater than 40 weight % (e.g., 40-60%, 50-70%, 60-90%, or 80-98%) (w/w) carboxylate salt of an LD prodrug dissolved in a liquid carrier. The liquid carrier can be a lipid (e.g., a triglyceride, a cholesterol ester, sesame oil, castor oil, or cottonseed oil), an alcohol (e.g., ethanol, glycerol or propylene glycol), N-methylpyrrolidone, or a mixture thereof. In particular embodiments the liquid carrier further includes an antioxidant. The liquid carrier can include water and a lipid. In particular embodiments, the liquid carrier includes an emulsion or liposomes. In certain embodiments, the pharmaceutical composition has at about 20° C. a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 100 cP, 50 cP to 500 cP, 250 cP to 750 cP, 500 cP to 1,000 cP, 750 cP to 2,000 cP, or 50 cP to 1,500 cP). In certain embodiments the pharmaceutical composition can remain substantially free of LD precipitate for at least 12 hours, 24 hours, 48 hours, or 72 hours when stored at about 25° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In particular embodiments the solubility of LD in the pharmaceutical composition is at least 5 g, 10 g, or 15 g per liter at about 25° C. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C.

In a related aspect, the invention features a method for treating Parkinson's disease in a subject by infusing into the subject a pharmaceutical composition including a liquid salt or fatty acid salt described above in an amount sufficient to treat Parkinson's disease.

The invention further features a method for treating Parkinson's disease in a subject by infusing into the subject a pharmaceutical composition including levodopa ethyl ester oleate, levodopa ethyl ester ricinoleate, levodopa ethyl ester palmitate, or levodopa ethyl ester valerate in an amount sufficient to treat Parkinson's disease.

The invention features a stable pharmaceutical composition suitable for injection into a subject including an aqueous liquid with LD prodrug particles, or a salt thereof, suspended therein, the LD prodrug particles having an effective particle size of from 20 nm to 1.0 μm (e.g., an effective particle size of from 20 nm to 0.5 μm, 100 nm to 1 μm, 150 nm to 800 nm, or from 150 nm to 600 nm). The LD prodrug particles include an LD prodrug, or a salt thereof.

In a related aspect, the invention features a method for treating Parkinson's disease in a subject by infusing into the subject a pharmaceutical composition including LD prodrug particles in an amount sufficient to treat Parkinson's disease.

In any of the above pharmaceutical compositions, the LD prodrug can be selected from LDAs, LDEs, LDCs, LDSs, and salts thereof. In particular embodiments, the LD prodrug is LDEE, LDME, or a salt thereof, such as LDEE hydrochloride salt.

The invention features a container including a pharmaceutical composition containing an LD prodrug described herein. In certain embodiments, the container includes an atmosphere substantially free of oxygen.

The invention features a container including a material that is substantially impermeable to oxygen, the container containing a reconstitutable solid including an LD prodrug, or a salt thereof, wherein the container is substantially free of oxygen and wherein the reconstitutable solid, when reconstituted, is suitable for subcutaneous infusion. The invention also features a container including a material that is substantially impermeable to oxygen, the container containing liquid including an LD prodrug, or a salt thereof, wherein the container is substantially free of oxygen and wherein the liquid is suitable for subcutaneous infusion. In particular embodiments, the container can further include a pharmaceutically acceptable excipient, such as a viscosity enhancing agent, an anti oxidant, and/or a preservative. For example, the container can further include from 0.5 to 4.0% (w/w) hyaluronic acid, or any other viscosity enhancing agent described herein; and/or the container can further include an antioxidant (e.g., bisulfite, propofol, ibuprofen, salicylic acid or salicylic acid salt, a salt of ascorbic acid (such as sodium ascorbate), p-aminophenol, acetamol, a t-butyl ortho-substituted phenol, or any antioxidant described herein).

In particular embodiments, the LD prodrug in the container is an LDE, or a salt thereof, such as an acid addition salt of LDEE (e.g., LDEE hydrochloride). In certain embodiments, the container is designed to hold less than 30 mL, 25 mL, 20 mL, 15 mL, 10 mL, 5 mL, 3 mL of a liquid including from 0.3 M to 4.0 M LD prodrug, or a salt thereof, (e.g., 0.4±0.1, 0.5±0.1, 0.6±0.1, 0.7±0.1, 0.8±0.2, 1.0±0.3, 0.8±0.3, 1.0±0.5, 1.5±0.5, 2.0±0.5, 2.5±0.5, 3.0±0.5, or 3.5±0.5 moles per liter) and having a pH of from 1.0 to 3.5 (e.g., 2.3±0.7, 1.8±0.3, 2.1±0.3, 2.5±0.3, or 2.7±0.3), wherein the container is substantially free of oxygen.

In a related aspect, the invention features a method for treating Parkinson's disease in a subject, the method including: (i) dissolving the solid contents of the container of the invention in buffering agent containing water to form an aqueous solution having a pH of from 4.0 to 6.0 (e.g., 4.2 to 5.0, 4.5±0.3, 4.4±0.2, 4.5±0.5 or 5.0±0.5) and an LD prodrug concentration of from 0.3 M to 4.0 M (e.g., 0.4±0.1, 0.5±0.1, 0.6±0.1, 0.7±0.1, 0.8±0.3, 1.0±0.5, 1.5±0.5, 2.0±0.5, 2.5±0.5, 3.0±0.5, or 3.5±0.5 moles per liter); and (ii) infusing the aqueous solution into the subject in an amount sufficient to treat Parkinson's disease. The buffered water can include a pharmaceutically acceptable potassium and/or a sodium salt of a dibasic, tribasic or tetrabasic acid, such as a salt of citric acid; pyrophosphoric acid, succinic acid, or phosphoric acid (e.g., trisodium citrate, tetrasodium pyrophosphate, disodium succinate, or trisodium phosphate). In particular embodiments, the LD prodrug is levodopa ethyl ester. In still other embodiments, the solution infused into the subject is substantially free of precipitated solids; has a pH of from 4.0 to 6.0 (e.g., 4.2 to 5.0, 4.5±0.3, 4.4±0.2, 4.5±0.5, or 5.0±0.25), and includes greater than 0.3 M (e.g., 0.5±0.2, 1.0±0.5 or 1.5±0.5, 2.0±0.5, 2.5±0.5, 3.0±0.5, 3.5±0.5, or greater than 3.5 moles per liter) levodopa ethyl ester.

In any of the above methods for treating Parkinson's disease, the method can be used to alleviate a motor or non-motor complication in a subject afflicted with Parkinson's disease, such as tremor, akinesia, bradykinesia, dyskinesia, dystonia, cognitive impairment, and disordered sleep. The method can further include administration of an effective amount of an anti-emetic agent (e.g., nicotine, lobeline sulfate, pipamazine, oxypendyl hydrochloride, ondansetron, buclizine hydrochloride, cyclizine hydrochloride, dimenhydrinate, scopolamine, metopimazine, or diphenidol hydrochloride). For example, the methods can include administering an effective amount of carbidopa or carbidopa prodrug (e.g., orally or by infusion). Examples of carbidopa prodrugs include its esters, such as carbidopa ethyl ester and carbidopa methyl ester, and carbidopa amide, the highly water soluble hydrochloride salts of which are preferred as carbidopa prodrugs. In either of the above methods, the pharmaceutical composition can administered by subcutaneous infusion or intramuscular infusion. For example, the pharmaceutical composition can be infused proximate a large muscle (e.g., the diaphragm, trapezius, deltoid, pectoralis major, triceps brachii, biceps, gluteus maximus, sartorius, biceps femoris, rectus femoris, and gastrocnemius) at a depth between 4 mm and 15 mm below the epidermis of the subject (e.g., 5 mm to 15 mm or 5 mm to 12 mm beneath the epidermis of the subject). In particular embodiments the pharmaceutical composition is coinfused with hyaluronidase and/or with an analgesic (e.g., salicylic acid, or a salt thereof; indomethacin; ibuprofen; amiloride; diclofenac; or calcium salts), or an analgesic is topically administered to the subject at the site of infusion.

The invention features an ambulatory infusion pump system for the treatment of Parkinson's disease including: (i) a pharmaceutical composition of the invention in a drug reservoir; and (ii) a cannula or needle in fluid communication with the drug reservoir for infusing the pharmaceutical composition into a subject. In particular embodiments, the pump system is a patch pump including an adhesive for adherence of the patch pump directly or indirectly to the skin of a subject. In one embodiment, the ambulatory infusion pump system can further include software, memory, a data processing unit, and information input/output capability, wherein the system is able to input, store and recall data including one or more of the subject's symptoms or drug responses related to Parkinson's disease, such symptoms selected from the group of tremor, hyperkinesia, dystonia, akinesia, bradykinesia, tremor, turning on, turning off, delayed time to on, and response failure. In a particular embodiment, the ambulatory infusion pump system can further include software, memory, a data processing unit, and user input capability to input into the system information related to the ingestion of a meal, and the system thereafter adjusts the rate of infusion of the pharmaceutical composition. For example, the pump system can be programmed to increase the rate of infusion after a meal including protein. In still other embodiments, the ambulatory infusion pump system can further include software, memory, a data processing unit, and information input/output capability, wherein the system is able to automatically increase the rate of infusion of the pharmaceutical composition, by a factor of two or more, at a preset time in the morning or after a period of at least four hours. In still another embodiment, the ambulatory infusion pump system can further include a data processing unit; and a motion sensor electrically connected to, or in RF communication with, the data processing unit to detect movement of the subject, wherein the system recommends a change in the infusion rate in response to the data from the motion sensor.

The invention features an ambulatory pump system for the treatment of Parkinson's disease including: (i) a first reservoir containing an acidic aqueous solution including from 0.3 M to 4.0 M (e.g., 0.4±0.1, 0.5±0.1, 0.6±0.1, 0.7±0.1, 0.8±0.2, 1.0±0.3, 1.0±0.5, 1.5±0.5, 2.0±0.5, 2.5±0.5, 3.0±0.5, or 3.5±0.5 moles per liter) LDEE, or a salt thereof; (ii) a second reservoir containing a basic aqueous solution; and (iii) a means for combining and a means for infusing the acidic aqueous solution and the basic aqueous solution into a subject (e.g., a cannula and/or needle in fluid communication with the first drug reservoir and the second drug reservoir for combining and infusing the acidic aqueous solution and the basic aqueous solution into a subject, optionally with a mixing chamber). In particular embodiments, the first reservoir contains an acidic aqueous solution having a pH of from 1.0 to 3.5 (e.g., 2.3±0.7, 1.8±0.3, 2.1±0.3, 2.5±0.3, or 2.7±0.3), and the second reservoir contains a basic aqueous solution having a pH of greater than 7.0 (e.g., greater than 7.5, 8.0, or 8.5). The acidic aqueous solution can include a pharmaceutical composition described herein. In particular embodiments, the basic aqueous solution includes a pharmaceutically acceptable potassium and/or a sodium salt of a dibasic, tribasic or tetrabasic acid, such as a salt of citric acid; pyrophosphoric acid, succinic acid, or phosphoric acid (e.g., trisodium citrate, tetrasodium pyrophosphate, disodium succinate, or trisodium phosphate).

The invention features a kit including (i) a pharmaceutical composition of the invention; and instructions for administering the composition to a subject for the treatment of Parkinson's disease.

The invention features a method for using the pharmaceutical composition of the invention, the method including the step of visually inspecting the composition prior to use to determine whether the pharmaceutical composition is suitable for infusion into a subject, wherein a transparent solution is suitable for infusion and a colored or opaque solution is not suitable for infusion. The pharmaceutical composition can be packed in a kit or container that is configured to permit visual inspection of the pharmaceutical composition.

The invention features a hydrolytically stable, oxidatively stable, pharmaceutical composition for reconstitution, including dry solid LDEE free base crystallites in a container including substantially no water and oxygen.

The invention features a compound of formula (II):

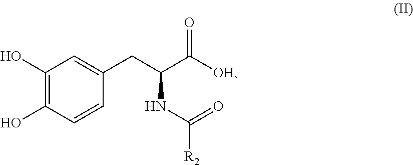

or a salt thereof, wherein R₂ is as defined herein. In particular embodiments, R₂ is CH₂CH₃, CH(OH)CH₃, CH₂CH₂COOH, CH₂CH₂CH₃, benzenepropenyl, phenyl, or (CHOH)₄CH₂OH.

The invention features a compound of formula (III):

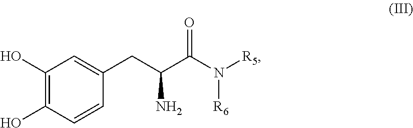

or a salt thereof, wherein R₅ and R₆ are as defined herein. In particular embodiments, R₅ is H or CH₃, and R₆ is CH₃, CH₂CH₃, CH₂CH₂CH₃, benzyl, 2-deoxy-2-glucosyl, or CH₂CH₂NH₂.

The invention features a compound of formula (IV):

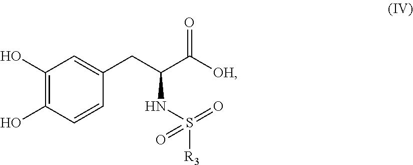

or a salt thereof, wherein R₃ is as defined herein. In particular embodiments, R₃ is CH₃, or 4-methylbenzyl.

The invention features a pharmaceutical composition including an aqueous liquid containing an LD prodrug, or a salt thereof, and water, wherein the weight percent of water in the pharmaceutical composition is less than the weight percent of said LD prodrug, or a salt thereof (e.g., by mass the ratio of LD prodrug, or a salt thereof, to water is from 1.05:1.0 to 1.25:1.0; 1.15:1.0 to 1.55:1.0; 1.25:1.0 to 1.75:1.0; 1.75:1.0 to 2.0:1.0; 1.85:1.0 to 3.0:1.0; or from 2.0:1.0 to 4.0:1.0).

The invention also features a pharmaceutical composition including an aqueous liquid containing an LD prodrug, or a salt thereof, and water, wherein the aqueous liquid has a density greater than 1.15 g/mL (e.g., a density of from 1.15 to 1.45, 1.25 to 1.65, or 1.35 to 1.95 g/mL) at about 25° C.

The invention further features a pharmaceutical composition including great than 0.25 M LD prodrug, or a salt thereof (e.g., 0.4±0.1, 0.5±0.1, 0.6±0.1, 0.7±0.1, 0.8±0.2, 1.0±0.3, 1.0±0.5, 1.5±0.5, 2.0±0.5, 2.5±0.5, 3.0±0.5, or 3.5±0.5 M LD prodrug); greater than 0.05 M carbidopa prodrug (e.g., 0.06±0.1, 0.7±0.1, 0.8±0.1, 0.9±0.1, 1.0±0.2, or 1.5±0.5 M carbidopa prodrug), or a salt thereof, and water. The pharmaceutical composition can optionally further include a vasoconstrictor.

In any of the above methods, the LD prodrug, or a salt thereof, can be infused intragastrically, intraduodenally or intrajejunally through a tube of less than about 3 mm, 2 mm, 1.5 mm, 1.0 mm outer diameter, and/or an internal diameter of less than 1 mm, 0.7 mm, 0.35 mm for a period of greater than or equal to about 12 hours, 24 hours, 48 hours, 72 hours, and most preferably 96 hours.

In any of the above methods, the LD prodrug, or a salt thereof, can be infused simultaneously at two or more infusion sites.

In any of the above methods, the LD prodrug, or a salt thereof, can be co-infused with a vasoconstrictor in an amount sufficient to reduce local swelling, inflammation, or nodule formation. The vasoconstrictor can be, without limtiation, a corticosteroid vasoconstrictor (e.g., dexamethasone, beclomethasone, clobetasol, betamethasone, fluocinolone, or derivatives, such as esters, thereof); or an adrenergic vasoconstrictor (e.g., neo-synephrine, phenylepinephrine, or oxymetazoline). In particular embodiments the vasoconstrictor is dexamethasone. The concentration of dexamethasone in the infused solution can be between about 1 μg/mL and about 4 mg/mL, and its preferred concentration is between about 10 μg/mL and about 1 mg/mL.

In any of the above pharmaceutical compositions, the pharmaceutical composition can include a vasoconstrictor in an amount sufficient to reduce local swelling, inflammation, or nodule formation when administered to a subject using a method described herein. The vasoconstrictor can be, without limtiation, a corticosteroid vasoconstrictor (e.g., dexamethasone, beclomethasone, clobetasol, betamethasone, fluocinolone, or derivatives, such as esters, thereof); or an adrenergic vasoconstrictor (e.g., neo-synephrine, phenylepinephrine, or oxymetazoline).

The invention further features a pharmaceutical composition including great than 0.25 M LD prodrug, or a salt thereof (e.g., 0.4±0.1, 0.5±0.1, 0.6±0.1, 0.7±0.1, 0.8±0.2, 1.0±0.3, 1.0±0.5, 1.5±0.5, 2.0±0.5, 2.5±0.5, 3.0±0.5, or 3.5±0.5 M LD prodrug); a vasoconstrictor (e.g., a corticosteroid vasoconstrictor or an adrenergic vasoconstrictor); and water. In particular embodiments, the vasoconstrictor is the corticosteroid vasoconstrictor dexamethasone. For example, the pharmaceutical composition can include between about 1 μg/mL and about 4 mg/mL dexamethasone (e.g., between 1 μg/mL and 0.5 mg/mL, 10 μg/mL and 1 mg/mL, 0.5 mg/mL and 2 mg/mL, or 1 mg/mL and 4 mg/mL). The pharmaceutical composition can optionally further include carbidopa, or a prodrug thereof.

Other features and advantages of the invention will be apparent from the following Detailed Description and the claims.

### DETAILED DESCRIPTION

The invention features pharmaceutical compositions, devices, systems, kits and methods for maintaining plasma LD concentrations in a desired therapeutic target range. The inventions enable PD patients to reduce the variability of their plasma and/or brain LD concentrations, e.g., reducing the magnitude and/or frequency of high or low LD excursions. By controlling the LD concentrations in the body, the durations of periods and/or severities of symptoms of PD, such as off periods and periods with severe dyskinesias, are reduced. The fluctuations are reduced by continuous, frequent and/or programmed, preferably subcutaneous or intramuscular, infusion of a solution of an LD prodrug. The concentration can be high enough to provide for daily infusion of less than 20 mL, 18 mL, 16 mL, 15 mL, 14 mL, 13 mL, 12 mL, 10 mL, 9 mL, 8 mL, 7 mL, 6 mL, 5 mL, 4 mL, 3 mL, or 2 mL of the LD prodrug containing solution. The preferred subcutaneously infused prodrugs include highly soluble LDEs, LDCs LDAs and LDSs and their salts, which can be rapidly hydrolyzed in the body, typically in an enzyme catalyzed reaction, to form LD, yet can be stored at least for the duration of the intended infusion period, for example at least 8 hours, 16 hours, 24 hours, 48 hours, 72 hours, in a container of the infusion system. For example, the infused solution can be an aqueous solution formed by mixing prior to use the storable contents of two containers or chambers. Optionally, one container contains an aqueous solution of the LD prodrug that can be stored with refrigeration at about 5±3° C., for example at about 4±2° C., for more than 3 months, 6 months, 12 months, 18 months, 24 months, 36 months, 48 months. The invention also features infusion pump systems for the administration of the formulations and methods of infusion.

The invention features a pharmaceutically acceptable, LD prodrug (e.g., LDA, LDE, LDC or LDS) formulation which can be stable in solution, which can be delivered via an ambulatory infusion pump, and which can provide continuous dopaminergic stimulation to the PD patient. The LD prodrug can be formulated to prevent rapid hydrolysis prior to its infusion, but can be rapidly hydrolyzed to form LD after its delivery into the body. Preferred LDEs are rapidly hydrolyzed in vivo by esterases. Preferred LDAs, LDCs and LDSs are rapidly hydrolyzed in vivo by amidases. Minimization of the symptoms of LD requires a relatively stable and sufficient plasma LD level, which could be achieved by careful consideration of the formulation of the LD prodrug and the rate of its continuous, or frequent or programmed infusion.

The prodrugs of the invention can be stored in liquid forms or solid forms, which can provide upon mixing of the contents of two containers or chambers an infusible aqueous solution prior to infusion into a subject.

The solubilities of the LDEs, LDCs, LDAs and LDSs can exceed the solubility of LD, the highest solubilities typically being observed for LD prodrug salt forms. For example, the solubilities of salts of LDEE and LDME, such as the salts formed when these bases are neutralized by HCl, are much more soluble than LD. The high solubility of LDEE.HCl allows for aqueous solutions of greater than 2.5 M in concentration. For example, a concentration of 3.0±0.5 M and a pH between about 1.0 and 3.5 that can be stored for long periods of time. The concentrated solutions can be stored and optionally diluted with up-adjusting their pH to be subcutaneously or intramuscularly infused, providing for continuous infusion therapy of PD. The infused volumes are typically much smaller than those intragastrically or intrajejunally infused.

The LD prodrugs are hydrolyzed to LD, which can be much less soluble in water or in aqueous solutions in the pH range suitable for subcutaneous or intramuscular infusion. The shelf life of the stored and of the infused (operational) LD prodrug solutions is usually determined by their hydrolysis, leading to LD precipitation, which can be faster than the other degradation processes, such as oxidation, particularly when oxygen is substantially excluded. For this reason, a major problem with the LD prodrug formulations, particularly of the aqueous formulations, is their hydrolytic instability. The rate of hydrolysis is pH and temperature dependent. Because the LD is poorly soluble, and because the concentration of the LD prodrug in the small-volume subcutaneously or intramuscularly infused solution is necessarily high, even hydrolysis of a small fraction of an LD prodrug or prodrug salt may result in the precipitation of LD from the solution. The presence of a large amount of LD precipitate is unacceptable, as it may lead to a dosing error and because it may block or reduce the flow in the infusion system.

At a particular temperature and pH, the LDEs formed of LD and of different alcohols are hydrolyzed prior to their infusion at different rates. For example, the rate of hydrolysis of LDEE near pH 7 and at about 37° C. can be about four times faster than the rate of hydrolysis of LDME. The rate of hydrolysis of LDE salts also depends on the anion, i.e., on the acid forming the LDE salt. The rate of hydrolysis of the salt formed of LDEE and acetic acid is about 3 times faster at about pH 4.5 at about 23° C. than that of LDEE.HCl, the salt formed of LDEE and HCl. The rate of hydrolysis at a particular pH and temperature also depends on the buffering agent, being slower at about pH 3 in citrate or phosphate buffered solutions than in the acetate buffered solution. The hydrolysis of LDE salts, such as LDEE.HCl, is strongly pH dependent. It is usually fast near neutral pH and decreases as the pH decreases until about pH 2; below about pH 1 it increases as the pH is further decreased. In strongly acidic solutions, e.g., of about pH 0.5 or less, the rate of hydrolysis is even faster.

Although precipitation of LD can be retarded or prevented by diluting the concentrated prodrug solution, such dilution defeats administration in the small volume required for subcutaneous or intramuscular administration. The shelf life can be very short for the typical LD prodrug aqueous solution at about neutral pH at an ambient temperature (e.g., 25° C.). To minimize hydrolysis and LD precipitation, the LDE salt can be stored in its dry solid form, and dissolved in water or in an aqueous solution prior to use. Alternatively and preferably, the LDE salt can be dissolved, and stored as an aqueous solution at a pH and at a temperature where the rate of hydrolysis is slow. The shelf life increases as the pH is lowered from neutral to the range from about pH 6 to about pH 5, increases further when the pH is lowered to the range from about pH 5 to pH 4, increases further when it is lowered from about pH 4 to about pH 3, and can be particularly long at about pH 2.3±0.7, for example at about pH 2.3. The operational life, meaning the life of the infused solution, is similarly pH dependent. The pH of a subcutaneously infused solution can be generally greater than about 4.0. The preferred operational pH range is between about 4.0 and about 6.0, the range between about 4.0 and 5.3 being more preferred; for example the pH of the infused solution can be 4.5±0.5 or 4.2±0.3. To extend the shelf life, the solution may be optionally stored at a temperature below about 25° C., for example it may be refrigerated at about 5±3° C. There would be no LD precipitation in an exemplary 2.5±0.5 M aqueous LDEE.HCl solution having a pH between about 1.5 and about 3.5 stored at about 5±3° C. (e.g., about 4° C.) for more than 1 year, or in an exemplary 2.5±0.5 M aqueous LDEE.HCl solution having a pH of 2.5±0.5 stored at about 5±3° C. (e.g., about 4° C.) for about 3 years. Upon raising the pH of the solution after 18 months of refrigerated storage to about 4.2±0.3 it would still remain precipitate free after more than 2 days at an operational temperature of 37° C., and for more than about 3 days at an operational temperature of 30° C.

Optionally, the aqueous LDE or LDC solutions may be stabilized by forming salts with polycarboxylic acids, the number of carboxylic acid functions exceeding the number of LDE or LDC amine functions. The aqueous liquid formulations of the invention can include an LDE or LDC formulated with one or more polycarboxylic acids (e.g., hyaluronic acid, succinylated gelatin, poly(acrylic acid), poly(methacrylic acid), poly(glutamic acid), poly(aspartic acid), poly(maleic acid), poly(malic acid), poly(fumaric acid), or a combination thereof. Other than for neutralizing the basic LDE or LDC, the polycarboxylic acid and/or its sodium salt can also be added to alter the viscosity of the solution, or as a crystal growth inhibitor, of an LD prodrug formulation (e.g., LDA, LDE, LDC or LDS).

For hyaluronic acid formulations, the preferred molecular weight average of the hyaluronic acid is from about 5,000 to about 2,000,000 Daltons (e.g., from 5,000 to 1,000,000; 5,000 to 850,000; 5,000 to 500,000; 50,000 to 850,000; 50,000 to 500,000; or from 150,000 to 850,000 Daltons). The formulation with hyaluronic acid can stabilize the LD prodrug (e.g., LDA, LDE, LDC or LDS) against rapid oxidation and/or hydrolysis, and can also produce a slow and controlled release when complexed (as a salt) to the hyaluronic acid. The concentration of the LD prodrug (e.g., LDA, LDE, LDC or LDS) complexed to the hyaluronic acid in the injectable slow-release solution would be typically between 0.5 and 2 moles per liter of the complexed LD ester (e.g., 0.5±5%, 1.0±5%, 1.5±4%, 2.0±5% moles per liter). The concentration for LD prodrug (e.g., LDA, LDE, LDC or LDS) complexed to other polycarboxylic acids can be the same. In general, in the LD prodrug (e.g., LDE or LDC) salt complexes with hyaluronic acid the ratio of hyaluronate carboxyl moiety to drug is greater than 1 (e.g., from about 1.1 to about 2.0, or from about 1.2 to about 1.8). For clarity, these are also the LDE molecule: hyaluronic mer ratios and the LDC molecule: hyaluronic mer ratios.

The inclusion of polycarboxylic acid in the formulations of the invention can reduce the local pain at the infusion site. Pain receptor controlled ion gates are opened by either high proton concentrations or by hyperosmotic solutions. Unlike monomeric acids, polymeric acids provide an internally strongly protonating environment, because of the high concentration of acidic functions within the macromolecule, yet they are much less ionized, because upon increasingly dissociating they acquire an increasingly negative charge, moderating the further release of protons. Also, when the LD prodrug (e.g., LDA, LDE, LDC or LDS) is polymer bound, the osmotic pressure is less than it would be if the LD prodrug (e.g., LDA, LDE, LDC or LDS) molecules were unbound.

The LD prodrug (e.g., LDA, LDE, LDC and LDS) formulations of the invention can be designed to enhance stability by reducing the rates of their hydrolysis, which usually dominates their degradation. While the dominant degradation process in the presence of water is hydrolysis, the LD prodrugs can also be oxidized by dissolved or gaseous oxygen. Exclusion of oxygen prevents such degradation. The products of the oxidation are not useful prodrugs, and in the absence of frequent monitoring (e.g., by HPLC or mass spectroscopy), oxidation makes accurate dosing difficult or impossible. The oxidation process is an autoxidation, meaning that a radical intermediate accelerates the oxidation, making it autocatalytic. The rate of oxidation can be reduced by several methods. One approach is to substantially exclude oxygen or reduce its partial pressure. The second is to include antioxidants, particularly pharmaceutically acceptable radical scavengers. Because ionization of catechol functions is an early step of the oxidation reaction sequence, oxidation can also be slowed or prevented by dissolving the LDA, LDE, LDS or LDC in a lipid, in which the catechol functions remain un-ionized because of the low dielectric constant. The third is to maintain a mildly acidic environment of a pH between about 2.3 and about 5.0, for example of about pH 4.5±0.5 or 4.2±0.3.

The daily required amounts of LD for PD management are generally between about 1.5 millimoles and 10 millimoles, typically between about 2.5 and 7.5 millimoles, and most often of about 5 millimoles. At the realized LD prodrug concentrations of >0.3M, >0.4M, >0.5M, >0.6M, >0.8M, >1.0 M, >1.5 M, >2 M, >2.5 M, >2.7 M in aqueous solutions or emulsions, the volumes can be small and can be infused subcutaneously or intramuscularly. Such high concentrations, particularly in non-aqueous solutions, and in emulsions, also allow reduction of the infused volume when the infusion is intragastric, intrajejunal or intraperitoneal.

The subcutaneous or intramuscular infusion of excessively acidic solutions is painful, triggering pain signals from acid sensing ion-flux gating neuronal receptors, and can also damage cells of the infused tissue, triggering an inflammatory reaction. Furthermore, the concentrated infused LD prodrug solutions can be hypertonic. Because infusion of excessively hypertonic solutions may also cause pain and cellular damage, the present invention features aqueous and non-aqueous compositions and methods for which infusion associated pain, cellular damage and inflammation can be reduced or avoided.

The aqueous liquid formulations of the invention can include the LD prodrug (e.g., LDA, LDE, LDC or LDS) formulated as a viscous liquid by including one or more viscosity enhancing agents in the formulation. The viscosity enhancing agents may also prevent or retard crystallization and precipitation of LD, e.g., from partially hydrolyzed LDA, LDE or LDC solutions. It can provide long lived, precipitate free, supersaturated LD prodrug solutions.

The viscosity of the infusible LD prodrug (e.g., LDA, LDE, LDC or LDS) compositions can typically be between about 1.2 cP and about 2,000 cp (e.g., between about 2 cp and 50 cP), when the viscosity is measured by glass capillary (Oswald) viscometer, or a falling sphere viscometer, or by a Brookfield viscometer, such as model LVDV-E of Brookfield Engineering Laboratories (11 Commerce Boulevard, Middleboro, Mass. 02346-1031 USA). The viscosity can by adjusted by an added sugar or a polyol, such as glucose, glycerol, sucrose, mannitol, or a polymer, such as hyaluronic acid, gelatin, collagen, dextran, albumin, polyethylene glycol (e.g., polyethylene glycol 3350), glycogen, succinylated gelatin, polylactic acid, polyglycolic acid, and DL-lactic and glycolic acids copolymers. The viscosity enhancing polymer can have a molecular weight average of between about 5,000 and about 2,000,000 Daltons. For example, the pharmaceutical compositions of the invention can include from 0.1 to 2.0% (w/w) hyaluronic acid having an average molecular weight of from 1×10⁶ to 2×10⁶ Daltons.

In general, the viscosities of the hyaluronic acid solutions increase with the hyaluronic acid concentration and with the molecular weight of the hyaluronic acid. In general, the preferred infused solutions including LD prodrug (e.g., LDA, LDE, LDC or LDS) have viscosities of less than about 10⁴ centipoise, preferably less than about 10³ centipoise, preferably between about 1.2 cp and about 2×10² cp at about 25° C. when the viscosity is measured with a glass capillary viscometer or by a falling sphere viscometer.

The aqueous liquid formulations of the invention can include the LD prodrug (e.g., LDA, LDE, LDC or LDS) formulated with one or more crystallization inhibitors, such as a sugar (e.g., hydroxyethyl starch, dextran, albumin, polyethylene glycol, mannitol, glucose), hyaluronic acid, succinylated gelatin, or other polycarboxylic acids.

The crystallization inhibitor (e.g., hyaluronic acid) can reduce the size of the precipitated LD crystallites or prevent the precipitation of crystallites of LD from its supersaturated solution. The size of precipitated crystallites is defined by the ratio of two rates: the rate of formation of crystal nuclei, the nucleation rate; and the rate of diffusion of the precipitated solute to the crystallites. Because the viscosity of the polymeric acid solutions and also of the concentrated saccharide (e.g., glucose) solutions is high, increasing with concentration and molecular weight, the dimensions of the precipitated crystallites, if any, can be made small enough to allow the pumping of their suspensions. In addition, the preferred adsorption of macromolecules on growing faces of crystallites prevents or reduces access of molecules of the precipitated solute, often fully preventing, or slowing, growth of the crystallites to dimensions where their surface/volume ratio is high enough for thermodynamic stability, the high surface energy de-stabilizing small crystallites (i.e., slowing the rate of nucleation or preventing nucleation).

Carbidopa Prodrugs

The invention also features a formulation including a carbidopa prodrug (e.g., carbidopa ester or carbidopa amide) which can be stable in solution, which can be delivered via an ambulatory infusion pump, and which can increase the LD half-life in the PD patient and/or reduce the daily LD or LD prodrug dose. It can be optionally co-dissolved and/or co-infused with the LD-prodrug. When co-infused with the LD prodrug the carbidopa prodrug can reduce the total daily infused LD dose. The carbidopa prodrug can be formulated to prevent rapid hydrolysis prior to its infusion, but is rapidly hydrolyzed to form carbidopa after its delivery into the body. Preferred carbidopa esters are rapidly hydrolyzed in vivo by esterases and preferred carbidopa amides are rapidly hydrolyzed in vivo by amidases. The prodrugs of the invention can be stored in liquid forms or solid forms, which can provide upon mixing of the contents of two containers or chambers an infusible aqueous solution prior to infusion into a subject.

The solubilities of the carbidopa esters and carbidopa amides can exceed the solubility of carbidopa, the highest solubilities typically being observed for carbidopa prodrug salt forms. For example, the solubilities of salts of carbidopa ethyl ester and carbidopa methyl ester, such as the salts formed when these bases are neutralized by HCl, are much more soluble than carbidopa. For example, the high solubility of carbidopa ethyl ester hydrochloride allows for aqueous solutions of high concentration. The concentrated solutions can be subcutaneously or intramuscularly infused, or they can be intragastrically or intrajejunally infused.

The carbidopa prodrugs are hydrolyzed to carbidopa, which can be much less soluble in water or in aqueous solutions in the pH range suitable for subcutaneous or intramuscular infusion. The shelf life of the stored and of the infused (operational) carbidopa prodrug solutions is usually determined by their hydrolysis, leading to carbidopa precipitation, which can be faster than the other degradation processes, such as oxidation, particularly when oxygen is substantially excluded. For this reason, a major problem with the carbidopa prodrug formulations, particularly of the aqueous formulations, is their hydrolytic instability. The rate of hydrolysis is pH and temperature dependent. Because the carbidopa is poorly soluble, and because the concentration of the carbidopa prodrug in the small-volume subcutaneously or intramuscularly infused solution is necessarily high, even hydrolysis of a small fraction of a carbidopa prodrug or prodrug salt may result in the precipitation of carbidopa from the solution. The presence of a large amount of carbidopa precipitate is unacceptable, as it may lead to a dosing error and because it may block or reduce the flow in the infusion system.

At a particular temperature and pH, the carbidopa esters formed of carbidopa and of different alcohols are hydrolyzed at different rates. For example, it is expected that the rate of hydrolysis of carbidopa methyl ester could be slower than the rate of hydrolysis of carbidopa ethyl ester. The rate of hydrolysis of carbidopa ester salts could also depend on the anion, i.e., on the acid forming the carbidopa ester salt. The rate of hydrolysis of the salt formed of carbidopa ethyl ester and acetic acid could be faster than the hydrolysis of the salt formed of carbidopa ethyl ester and HCl. The rate of hydrolysis at a particular pH and temperature also depends on the buffering agent, being slower at about pH 3 in citrate or phosphate buffered solutions than in the acetate buffered solution. The hydrolysis of carbidopa ester salts, such as carbidopa ethyl ester hydrochloride, is expected to be strongly pH dependent. It is expected to be fast near neutral pH; to decrease as the pH decreases until about pH 2; below about pH 1 it is expected to increase as the pH is further decreased. In strongly acidic solutions, e.g., of about pH 0.5 or less, the expected rate of hydrolysis is even faster.

Although precipitation of carbidopa can be retarded or prevented by diluting the concentrated prodrug solution, excessive dilution defeats administration in the small volume required for subcutaneous or intramuscular administration. The shelf life can be very short for the typical carbidopa prodrug aqueous solution at about neutral pH at an ambient temperature (e.g., 25° C.). To minimize hydrolysis and carbidopa precipitation, the carbidopa ester salt can be stored in its dry solid form, and dissolved in water or in an aqueous solution prior to use. Alternatively, the carbidopa ester salt can be dissolved, and stored as an aqueous solution at a pH and at a temperature where the rate of hydrolysis is slow. The shelf life is expected to increase as the pH is lowered from neutral to the range from about pH 6 to about pH 5, increase further when the pH is lowered to the range from about pH 5 to pH 4, increase further when it is lowered from about pH 4 to about pH 3, and can be particularly long at about pH 2.3±0.7, for example at about pH 2.3. The operational life, meaning the life of the infused solution, is similarly pH dependent. The pH of a subcutaneously infused solution can be generally greater than about 4.0. The preferred operational pH range is between about 4.0 and about 6.0, the range between about 4.0 and 5.3 being more preferred; for example the pH of the infused solution can be 4.5±0.5 or 4.2±0.3. To extend the shelf life, the solution may be optionally stored at a temperature below about 25° C., for example it may be refrigerated at about 5±3° C. No carbidopa precipitation is expected in an exemplary 1.0±0.5 M aqueous carbidopa ethyl ester hydrochloride solution when having a pH between about 1.5 and about 3.5 and stored at about 5±3° C. (e.g., about 4° C.) for more than 1 year, or when having a pH of 2.5±0.5 and stored at about 5±3° C. (e.g., about 4° C.) for about 3 years. Upon raising the pH of the solution after 18 months of refrigerated storage to about 4.2±0.3 it would still remain precipitate free after more than 2 days at an operational temperature of 37° C., and for more than about 3 days at an operational temperature of 30° C.

The daily required amounts of carbidopa for PD management are generally between about 0.3 millimoles and 3 millimoles, typically between about 0.6 and 2.0 millimoles, and most often of about 1-2 millimoles. At concentrations of >0.3 M, >0.4 M, >0.5 M, >0.6 M, >0.8 M, >1.0 M, >1.5 M, >2 M, >2.5 M, >2.7 M in aqueous solutions or emulsions, the volumes can be small and can be infused subcutaneously or intramuscularly. Such high concentrations, also allow reduction of the infused volume when the infusion is intragastric, intrajejunal or intraperitoneal.

Antioxidants

LD and LD prodrugs (e.g., LDA, LDE, LDC or LDS) can be susceptible to oxidative degradation. To minimize oxidative degradation the formulations of the invention optionally contain one or more antioxidants. Antioxidants that can be used in the aqueous formulations of the invention can be selected from thiols (e.g., dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, lactoferrin, citric acid, lactic acid, and malic acid, EDTA, EGTA, and DTPA); or reducing agents, such as sodium metabisulfite, vitamin C, sodium ascorbate, ascorbyl palmitate, Mg ascorbyl phosphate, and ascorbyl acetate), phenols, uric acid, or combinations thereof. The total amount of antioxidant included in the formulations can be from 0.01% to 2% by weight.

For use in the non-aqueous liquid formulations or emulsions, the LD prodrug can be formulated with one or more antioxidants selected from vitamin E; beta-carotene; tert-butylhydroxytoluene, tert-butylhydroxyanisole, ubiquinol, nordihydroguaiaretic acid trihydroxybutyrophenone, benzoates like coniferyl benzoate, terTBHQ (tert-butyl hydroquinone), propylgallate (3,4,5-trihydroxybenzoate), and dodecyl gallate, or a mixture thereof. The total amount of antioxidant included in the formulations can be from 0.01% to 2% by weight.

Particulate Containing Formulations

In an alternative approach, the pharmaceutical formulations described herein can include non-precipitating particles of an LD prodrug (e.g., LDA, LDE, LDC or LDS), or a salt thereof typically having an effective particle size of less than about 1 micron (i.e., nanoparticulate including formulations). These LD prodrug particles can be made by using any method known in the art for achieving the desired particle sizes. Useful methods include, for example, homogenization, supercritical fluid fracture, or precipitation techniques. Exemplary methods are described in U.S. Pat. Nos. 4,540,602; 5,145,684; 5,518,187; 5,718,388; 5,862,999; 5,665,331; 5,662,883; 5,560,932; 5,543,133; 5,534,270; and 5,510,118; 5,470,583, each of which is specifically incorporated by reference.

LD Prodrugs

The invention features compositions, methods, and infusion pumps for infusing an LD prodrug and or its salt. The LDEs are hydrolyzed in vivo to an alcohol; the LDCs are hydrolyzed in vivo to LD and a salt, mostly sodium salt, of a carboxylic acid; the LDAs are hydrolyzed in vivo to LD and an ammonium salt, mostly an ammonium chloride; and the LDS are hydrolyzed in vivo to LD and a sulfonate salt, mostly sodium sulfonate salt. In general, the oral, i.e., ingested LD₅₀ of the produced alcohol or sodium carboxylate, or ammonium chloride, or sodium sulfonate is greater than 3 millimoles/kg.

LDEE can be prepared from LD and ethanol, for example, as described in PCT Publication Nos. WO2003/042136 and WO2000027801; as described in U.S. Pat. Nos. 5,525,631; 6,218,566, and/or 5,354,885; or as described by Marrel et al., European Journal of Medicinal Chemistry, 20:459 (1985), each of which is incorporated herein by reference. Other esters of LD can be prepared from LD and the corresponding alcohol using analogous synthetic methods.

In aqueous LDE salt solutions, the hydrolysis rates generally decrease as the pH decreases, and the shelf life of the LDE salt consequently increases, unless the pH is about 1.0±0.5 or less. Thus at about pH 2.3 the LDEE.HCl solutions are generally more stable than at about pH 3; at pH 3 the LDEE.HCl solution are generally more stable than at about pH 4; at about pH 4 they are generally more stable than at about pH 5; at about pH 5 they are generally more stable than at about pH 6; and at about pH 6 they are generally more stable than they are at about pH 7. In acidic solutions the amines of the LDEs are protonated, making the LDEs cations. The rates of hydrolysis of the protonated LDE cations depend on the charge-balancing anion. In general, the rates of hydrolysis for salts with anions formed by the dissociation of weak acids are greater than those formed by the dissociation of strong acids. For example, the acetate of the protonated LDEE salt may hydrolyze about three times more rapidly than its chloride salt. At neutral pH, LDEE is hydrolyzed within hours or less, making the pH 7 solution unsuitable for most infusion situations. The rates of hydrolysis generally increase with temperature, and may at least about double or about triple for each 10° C. increase, correspondingly decreasing upon cooling. When a buffer is added to maintain a particular pH the anion or anions of the buffer affect the rate of hydrolysis. For example, at a pH of about 2.3, hydrolysis in the presence of acetate buffer is more rapid than in the presence of citrate buffer or phosphate buffer, wherefore the use of citrate buffer or phosphate buffer is preferred.

The infused pharmaceutical compositions may include LDA, LDC and/or LDS. The LDCs can be synthesized using the methods described by Zhou et al., European Journal of Medicinal Chemistry, 45:4035 (2010).

LD prodrugs can be prepared from LD in a process that may include the selective protection and deprotection of the hydroxyl, amine, and/or carboxyl functional groups of the LD. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for hydroxyl groups include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, hydroxyl, and carboxyl functionalities and the conditions required for their removal are provided in detail in T. W. Green and P. G. M. Wuts, Protective Groups in Organic Synthesis (2nd Ed.), John Wiley & Sons, 1991 and P. J. Kocienski, Protecting Groups, Georg Thieme Verlag, 1994.

LD-treated people with advanced PD require typically daily 0.5-1.5 g (2.5-7.5 millimoles) oral LD. The intent is to subcutaneously infuse the prodrug equivalent of the mid-range 1 g LD in a volume of preferably less than 20 mL, 18 mL, 16 mL, 15 mL, 14 mL, 13 mL, 12 mL, 11 mL, 10 mL, 9 mL, 8 mL, 7 mL, 6 mL, 5 mL. Lesser volumes of 4 mL, 3 mL, or less than 2 mL can be used when the infusion is intragastric, intraduodenal or intrajejunal. The respective concentration for 1 g (5 millimoles) equivalent of any LD prodrug is 0.3 M when the infused volume is 20 mL, 0.5 M when the infused volume is 10 mL, 1.0 M when the infused volume is 5 mL. When the infusion is intragastric, intraduodenal or intrajejunal the concentrations can be higher, 1.67 M when the infused volume is 3 mL, 2.5 M when the infused volume is 2 mL, and 3.0 M when the infused volume is 1.67 M.

At the higher concentrations, the weight percentage of water is less than the weight percentage of the prodrug, yet the solutions are clear, homogenous, liquids. For example, the weight % of water in the 2.6 M LDEE.HCl solution is about 42 weight %, and that in the 2.9 M solution is about 36 weight %. Their densities can be high, in excess of 1.15 g/mL at 25.0 for the more concentrated solutions. For example, the density of the 2.6 M LDEE.HCl solution is about 1.17 g/mL and that of the 2.9 M LDEE.HCl solution it is about 1.19 g/mL.

The preferred anion of the LDE salts or the LDC salt is the chloride ion, the only anion present in body fluids at >0.1 M concentration, because infusion of 5 millimoles of its salt does not substantially affect its systemic concentration. For this reason, the preferred anion is chloride, i.e., in the case of LDEE the preferred salt is LDEE.HCl. For the same reason, the preferred cation in the LDA and LDS salts is the sodium cation.

The LD prodrug (e.g., LDE or LDC) may be administered in its free base form or as a pharmaceutically acceptable salt, preferably its chloride salt. It may be administered also as a salt with an anion known to be very rapidly metabolized through cycles, such as the Krebs cycle (e.g., lactate, acetate, citrate, gluconate, malate, malonate, fumarate, succinate, isocitrate, or 1-glycerophosphate). Of these, lactate, present in blood and interstitial fluid at >1 mM concentrations is preferred. In certain instances the formulation of the invention includes a hydrochloride salt of an LD prodrug (e.g., LDA or LDE).

In certain instances, that are particularly relevant to intragastric, intraduodenal or intrajejunal infusion, the formulation is a non-aqueous formulation or emulsion of the invention that includes a carboxylate salt of an LD prodrug (e.g., LDA or LDE). The anions of these salts are typically aliphatic or aromatic carboxylate anions, such as eicosapentaenoate, docosahexaenoate, ricinoleate, alpha-linolenate, gamma linolenate, dihomo-gamma linolenate, arachidonate, linoleate, myristate, oleate, palmitate, palmitoleate, and/or stearate and may have an odd or even number of carbon atoms. The preferred carboxylate anions are aliphatic and have an even number of carbon atoms between 8 and 22, more preferably between 8 and 22, e.g. 12 and/or 14 and/or 16 and/or 18 and/or 20. They are preferably mono-unsaturated, and are more preferably poly-unsaturated. Those with cis double bonds are preferred over those having trans double bonds. Examples of the anions include arachidonate, linoleate, palmitoleate, and/or oleate.

The equilibrium reaction in which the free base LD prodrug is reacted with a carboxylic acid to form an acid addition salt (e.g., via proton transfer from a carboxylic acid to an amine) can result in a mixture that can contain some fraction of free LDE or LDA and free carboxylic acid. Using an excess of carboxylic acid to form the acid addition salt can be advantageous for slightly lowering the local pH and thereby stabilizing the catechol of the ester against oxidation by dissolved oxygen and also for lowering the temperature of the liquidus. Typically the molar excess of the carboxylic acid is about 10 mole % or less.

The shelf lives of the LD prodrug salts are generally limited by hydrolysis in the presence of water, e.g., in aqueous solutions and also in humid atmospheres; and by their air-oxidation. By using liquid LD prodrug salts or salt mixtures (e.g., liquid salts) instead of aqueous solutions the rates of hydrolysis can be greatly reduced, and the concentration of the prodrug or prodrugs is increased, reducing the volume of the liquid that needs to be subcutaneously or intramuscularly infused. The liquid LD prodrug salt formulations can be advantageously difficult to oxidize. The process is an autoxidation, meaning that a radical intermediate accelerates the oxidation, making it autocatalytic. The rate of oxidation can be reduced by several methods. One approach is to exclude oxygen. The second is to include anti-oxidants, particularly pharmaceutically acceptable radical scavengers capturing radical intermediates involved in the oxidation of the catechol functions of the LD prodrugs. These include for example capture by the polyunsaturated aliphatic carboxylates of the composition, by added vitamin E, and by added tert-butylphenols. The third is to increase the viscosity (relative to that of water). At the higher viscosity of the oils (relative to water) the rate of oxidation by dissolved oxygen is reduced. The oxidation is also slowed or prevented in the liquid LD prodrug salts because their dielectric constants are lower than those of aqueous solutions. Ionization of the catechol functions is usually the first step in their oxidation; at the lower dielectric constants of the liquid LD prodrug salts, the catechol functions are less ionized.

The acid addition salts of the invention that are of particular relevance to intragastric, intraduodenal or intrajejunal infusion, can be formulated with lipids, such as carboxylic acids, and mono, di or triglycerides of carboxylic acids, and/or other esters of C₁₂-C₂₂ carboxylic acids, preferably esters melting below 25° C., such as ethyl myristate and ethyl oleate. The preferred carboxylic acids and their glycerol or ethanol esters are those with 12 and/or 14 and/or 16 and/or 18 and/or 20 carbon atoms; monounsaturated are preferred over saturated; polyunsaturated are preferred over monounsaturated; and cis-mono-unsaturated or cis-polyunsaturated acids are preferred over the trans-acids. The lipid excipients are typically mixtures, i.e., like sesame oil, castor oil, or linseed oil, and/or carboxylic acids, like oleic, linoleic, or palmitoleic acid and can be combined with alcohols, such as glycerol.

When the fatty acid LD salts or the oils are metastable liquids, meaning that they are liquids that can eventually crystallize, their crystallization is retarded when the viscosity is increased. For their storage, it is convenient to increase the viscosity by refrigeration at about 5±3° C. (e.g., about 4° C.) the temperature of many refrigerators, or at about −18° C., the temperature of many freezers.

LDEE.HCl and the Stabilization of its Aqueous Solutions

We have found that the solubility of LD increases remarkably with the concentration of LDEE.HCl. For example, we have found that in a citrate buffered solution of about pH 4.5 not containing LDEE.HCl the solubility of LD at 25° C. is about 0.68 g/100 mL or 34 mM. In a citrate buffered solution of about pH 4.5 containing 1.3 M LDEE.HCl the solubility of LD at 25° C. is about 1.0 g/100 mL or 51 mM. In a citrate buffered solution of about pH 4.5 containing about 2.6 M LDEE.HCl the solubility of LD at 25° C. is about 1.7 g/100 mL or 86 mM. Because of the increased solubility of LD in the citrate buffered LDEE.HCl solutions, saturation of the formulated drug solution resulting in undesired LD precipitation is delayed when the LDEE.HCl is hydrolyzed to LD, ethanol, and acid, the acidification further increasing the solubility and delaying precipitation. The hydrolytic stability of concentrated aqueous solutions of LDEE.HCl is best between about pH 2.0 and about pH 3, and it is preferred to store the solutions at pH 2.3±0.7. Such a pH can be maintained for example through co-dissolving citrate, e.g., as trisodium citrate, for example to about 2-50 mM concentration, typically to about 10-40 mM concentration, and preferably to 20-35 mM concentration. For infusion, it is desired, in order to avoid acid-caused pain, to raise the pH at least to pH 4.0±0.5, preferably pH 4.8±0.8, for example, by adding a solution of trisodium citrate. Exemplary estimated storage and operational lives are provided in Table 1 for an about 2.0 M LDEE.HCl solution.

TABLE 1

| | |
|---|---|
| Infused volume (per 1 g LD equivalent), mL | 1.9 |
| Estimated pH 2.3 storage life at 4° C., months | 50 |
| Estimated pH 2.3 storage life at 25° C., months | 2 |
| Estimated pH 4.5 operational life at 30° C., days | 4 |
| Estimated pH 4.5 operational life at 37° C., days | 2 |

The burden of hypertonicity can be reduced by increasing the daily administered volume (for the administration of 1 g LD equivalent) to about 5-20 mL.

Infusion Pumps

The pharmaceutical compositions of the invention, optionally in combinations with other drugs used for the treatment of PD, such as enzyme inhibitors like carbidopa, or a prodrug of carbidopa such as its ethyl ester hydrochloride or its methyl ester hydrochloride, can be infused, preferably subcutaneously or intramuscularly, using an infusion pump, which can optionally be a syringe-type infusion pump. The pump can be configured to automatically infuse continuously or intermittently, and/or administration can be subject-controlled.

Any suitable type of infusion pump may be used to deliver the LD prodrug (e.g., LDA, LDE, LDC or LDS) including liquid composition. These may include implantable and non-implantable pumps, pumps for intramuscular, subcutaneous, percutaneous, or intrathecal delivery, fixed position or ambulatory pumps, patch pumps and carried pumps. These pumps may employ any pump drive mechanism known in the art including syringe, hydraulic, gear, rotary vane, screw, bent axis, axial piston, radial piston, peristaltic, spring-driven, gas-driven, piezo-electric, electroosmotic, and wax expansion. For example, for infusing large volumes, an infusion pump can include a peristaltic pump. Alternatively, for infusing small volumes, an infusion pump can include a computer-controlled motor, turning a screw that pushes the plunger of a syringe.

An intrathecal pump can be used to deliver very small quantities of a pharmaceutical composition directly to the intrathecal space and cerebrospinal fluid of a subject. Intrathecal pumps are typically implanted in the body, with a catheter leading from the pump to the target location. Such pumps typically have a drug reservoir refillable via a drug injection, have a battery life of about six years, and have a capacity of about 20 mL or 40 mL of drug. Their pumps are typically peristaltic. Flow rates vary from about 0.048 mL/day to 24 mL/day. The flow rate accuracy of the pump is typically within +/−14.5% of the programmed flow rate at 0.048-24 mL/day at 37° C., 50% reservoir volume, and 300 meters above sea level. Such pumps are typically programmable and the infusion rates can be modified non-invasively.

Ambulatory drug infusion pumps can be used for subcutaneous or intravenous administration of a pharmaceutical composition of the invention. One example of an ambulatory infusion pump used to treat PD is the Smiths Medical CADD-Legacy 1400 ambulatory pump, which is used to deliver the Duodopa gel. The pump is reusable and works with a disposable cassette containing the drug. The cassette has a 100 mL reservoir containing 20 mg/mL LD and 5 mg/mL carbidopa in a gel; carmellose sodium is used as a thickening agent. The shelf life is 15 weeks when refrigerated, and 24 hours at room temperature. The Duodopa gel is infused from the extracorporeal pump to the duodenum through a catheter that is surgically implanted through the wall of the abdomen in a percutaneous gastrostomy operation.

Some features of the CADD-Legacy pump include a display, cassette detection, occlusion detection, air-in-line detection, on/off key, event memory and programmable infusion rates. The infusion regimen suggested in the Duodopa users guide includes a morning dose (administered when the subject wakes up in order to quickly achieve the concentration required for optimal subject response); a continuous maintenance dose (administered continuously by the pump to maintain a constant circulating concentration); and extra doses (administered if the subject experiences reduced mobility during the day).

Another example of an ambulatory infusion pump is the APO-go pump for infusion of apomorphine, a dopamine agonist. It is indicated for the treatment of disabling motor fluctuations (‘on-off’ phenomena) in subjects with PD. The pump infuses apomorphine 10 mg/mL solution.

A particular class of ambulatory drug infusion pumps, which can be used for the delivery of the pharmaceutical compositions of the invention, are single, two and multi-compartment pumps designed to infuse drugs, for example insulin to patients with diabetes. These can generally be broken down into two groups: skin-attached “patch pumps” and carried pumps. Examples of insulin patch pump designs by various companies include those described in U.S. Pat. Nos. 7,914,499, 7,806,867; 7,740,607; 7,530,968; 7,481,792; 7,771,412; 7,303,549; 7,144,384; 7,137,964; 7,029,455; 7,018,360; 6,960,192; 6,830,558; 6,768,425; 6,749,587; 6,740,059; 6,723,072; 6,699,218; 6,692,457; 6,669,669; 6,656,159; 6,656,158; 6,485,461; 7,815,609; 7,771,391; 7,713,262; 7,713,258; 7,632,247; 7,520,295; 7,517,335; 6,726,655; 6,669,668; 6,428,518; 6,416,496; 6,146,360; and 6,074,366, and U.S. Patent Publication Nos. 20110137287, 20100217191; 20100274218; 20100243099; 20080319416; 20080319414; 20080319394; 20080319384; 20080255516; 20080234630; 20080215035; 20070191702; 20100137784; 20070250007; 20060206054; and 20090320945, each of which is incorporated herein by reference. Examples of carried pump designs by various companies include those described in U.S. Pat. Nos. 6,551,276 and 6,423,035, each of which is incorporated herein by reference. The preferred pumps are inexpensive, optionally single-use, skin attached patch pumps, optionally with two compartments. One, two or more inexpensive patch-pumps can be attached to the skin in order to increase the dose rate or the dose of the LD-prodrug, or to better distribute the infused volume.

An exemplary useful pump is the Crono syringe-type programmable infusion pump of Cané s.r.l. Medical Technology Via Pavia 105/I Rivoli (TO) Italy. Its dimensions are 77×48×29 mm (3×1.9×1.1 inch) and its weight is 115 g, its 3 Volt type 123 A lithium battery included. The capacity of its syringe is 10 or 20 mL. The delivered solution volume can be programmed from 1 to 20 mL for delivery times from 30 minutes to 99 hours, in 15 minutes steps. The accuracy is +/−2%. The occlusion pressure is 4.5±1 bar. The pump is programmable and the data are automatically stored in the pump's memory; in the event of an anomaly, an alarm is provided and an error message is displayed. The pump's functions can be “locked” such that the subject will not accidentally change a function by pushing a button. The pump operates accurately in the 10° C.-45° C., at 30%-75% relative humidity and through the 700 hPa-1060 hPa (hectopascal) atmospheric pressure range.

Yet another exemplary useful pump that can be used in the methods and devices of the invention is an electro-osmotic drug pump, such as that described in PCT Publication No. WO 2011112723; W. Shin et al., Drug Delivery and Translational Research 1:342 (2011); W. Shin et al., Journal of the American Chemical Society 133, 2374 (2011); and in W. Shin et al., Analytical Chemistry 83(12), 5023 (2011).

The pumps preferred are externally worn and infuse subcutaneously or intramuscularly and can infuse solutions of 1 cP, 10 cP, 100 cP, 1000 cP viscosity at about 30.0 at average rates of more than 1 μL per min, preferably at least 2, 5, 10 μL per minutes.

Infusions may be made continuously or intermittently, with sample intermittent infusion intervals being less than or equal to about every 5, 10, 15, 30, 60, 90 or 120 minutes.

Infusion rates may be set to one or more values that equates to a rate of LD prodrug (e.g., LDA, LDE, LDC or LDS) delivery of anywhere between 1-200 mg/hr. For subcutaneous or intramuscular delivery representative rates may be between 10-100 mg/hr. Sample infusion rates may equate to about 10, 20, 30, 40, 50, 60, 70, 80, 90, 100, 125, 150, 175 or 200 mg/hr of LD prodrug (e.g., LDA, LDE, LDC or LDS). For intrathecal delivery lower rates may be used; sample rates may equate to less than or equal to about 10, 5, 1, 0.5 and 0.1 mg/hr of LD prodrug (e.g., LDA, LDE, LDC or LDS).

Pump flow rates depend on the concentration of the LD prodrug (e.g., LDA, LDE, LDC or LDS) in the solution. Convenient flow rates range from less than or equal to about 1.4, 1.2, 1.0, 0.8, 0.6, 0.4, 0.3, 0.2, 0.1 or 0.04 mL/hr. For intrathecal pumps flow rates typically vary from about 0.0048 mL/day to 2.4 mL/day. Sample flow rates equate to less than about 20, 15, 10, 5, 1, 0.5, 0.1, and 0.01 mL/day.

LD prodrug (e.g., LDA, LDE, LDC or LDS) from a single container may be infused s.c. or intramuscularly by the pump for a period of greater than or equal to about 8 hours, 12 hours, 16 hours, 24 hours, 48 hours, 72 hours or 96 hours. The container may contain the equivalent of between 0.25-20 g of LD prodrug (e.g., LDA, LDE, LDC or LDS), or of 1-6 g of LD prodrug (e.g., LDA, LDE, LDC or LDS). Examples of the equivalent amounts of LD prodrug (e.g., LDA, LDE, LDC or LDS) that may be contained in a container are about 0.5±0.2, 1±0.3, 2±0.4, 3±0.5, 4±0.7, 5±0.8, 6±2, 8±2, 10±2, 12±2, 14±2, 16±2, 18±2, or 20±2 g. Implantable pumps may contain greater amounts of drug in their reservoirs.

Any suitable type of infusion pump may be used to subcutaneously or intramuscularly deliver the non-aqueous compositions or the emulsions. These may include implantable and non-implantable pumps, fixed position or ambulatory pumps, patch pumps and carried pumps. The pumps preferred are externally worn and infuse subcutaneously or intramuscularly and can infuse solutions of 1 cP, 10 cP, 100 cP, 1000 cP viscosity at about 30° C. at average rates of more than 0.1 mL/hr, preferably at least 0.2 mL/hr, 0.3 mL/hr, 0.4 mL/hr, 0.5 mL/hr, 0.6 mL/hr. Typical infused LD prodrug dose ranges are from about 10 micromoles per kg of subject weight to about 200 micromoles per kg of subject weight of LD prodrug per day. For example, the typical daily dose for a subject weighing 75 kg is from about 0.75 millimoles to about 15 millimoles of LD prodrug. Infusion rates may be set to one or more values that equates to a rate of LD prodrug delivery of anywhere between about 30 micromoles per hour and about 600 micromoles per hour. For an exemplary solution in which the concentration of the LD prodrug is 0.5 M, these values correspond to average flow rates of 60 and 1,200 microliters per hour respectively. The exemplary dosage/kg of LD prodrug to be administered is likely to depend on such variables as the stage of the PD of the subject, the dose/kg being higher for subjects in more advanced stages of the disease and on the particular formulation of the LD prodrug being used. In continuous operation, the preferred pump flow rate is between about 0.2 mL per hour and about 1.5 mL per hour. In intermittent operation the flow rate depends on the duty cycle. For example, if the pump is on for 10 min and is off for 20 min the pumping rate while the pump is on is between 0.6 mL per hour and about 4.5 mL per hour. LD prodrug from a container may be infused s.c. or intramuscularly by the pump for a period of greater than or equal to about 8 hours, 12 hours, 16 hours, 24 hours, 48 hours, 72 hours or 96 hours. The container may contain between about 1 millimole and about 0.1 mole of LD prodrug. An about 10 mL exemplary container, typically replaced daily, may contain about 7 millimoles of the LD prodrug containing enough prodrug to form about 1350 mg LD when hydrolyzed in-vivo by an esterase.

In one embodiment the flow rate is constant rather than being adjusted by the user or health care provider. Pumps with different constant flow rates are to be provided for users requiring different daily doses of the LD prodrug. In another embodiment the flow rate is constant for all users, and the users are provided with solutions of different LD-prodrug concentrations. Advantages of the fixed flow rate pumps include their low cost and the simplicity of their use.

Pumps of the present invention can include some or all of the following elements: a pump drive mechanism; a subcutaneous or intramuscular infusion set, cannula, or needle; an intrathecal catheter (e.g., for delivering dopamine); an inserter for the subcutaneous or intramuscular infusion set, cannula, or needle; a drug reservoir; a display; an input mechanism (e.g., a keypad or touchscreen); a memory; a remote control; a data processing unit; an alarm; a battery; a transmitter; a receiver; an occlusion sensor; data download or transmission capability; the ability to input disease-related data (e.g., event markers, sensor measurements, meals, exercise, etc.); algorithms to recommend or control drug basal and/or bolus dosing; and an adhesive to attach to the skin or a clip to attach to clothes. The pumps can be configured to obtain data from sensors through a physical or a wireless connection, or even from physical integration of the units. The pump may also be configured to communicate with tremor or movement monitoring accelerometers, computers, cell phones, the internet and various communication networks.

The drug reservoir(s) can be equipped with a septum, which is penetrated to provide fluid contact between the reservoir and the infusion needle. The septum of the reservoir can be made from a polymer having low oxygen permeability, such as polyvinylidene chloride, filler loaded butyl rubber (poly(isobutylene-co-isoprene)), filler loaded chlorobutyl rubber, chlorobutyl rubber, bromobutyl rubber, butyl rubber, chlorosulfonated polyethylene (Hypalon), or amorphous polyethylene terephthalate.

The infusion set's catheter can be constructed to have low permeability to oxygen. The catheter can, optionally, be long, e.g. 60 cm; its ID can be typically less than 1 mm (e.g., about 0.7 mm, about 0.4 mm, or less). Its wall thickness can be less than 1 mm and greater than about 0.2 mm (e.g., between about 0.4 and about 0.6 mm). The catheter can be optionally formed from a polymer, such as polyvinylidene chloride, filler loaded butyl rubber (poly(isobutylene-co-isoprene)), filler loaded chlorobutyl rubber, chlorobutyl rubber, bromobutyl rubber, butyl rubber, chlorosulfonated polyethylene (Hypalon), or amorphous polyethylene terephthalate.

Simple, low cost pumps may be entirely or partially disposable after use. Non-programmable pumps may simply deliver a constant basal infusion rate; optionally, they may also have the ability to deliver a fixed bolus or multiple fixed boli on command.

The pump can include the software, memory and hardware to enable the pump to input, store, recall, display, communicate and/or analyze event markers useful to management of PD. Such event markers can include: (i) intake of the infused medications, including dose and time; (ii) intake of other PD medications, including identification of the drug, dose and time (e.g., such medications may include DDC inhibitors, dopamine receptor agonists, MAO-B agonists, COMT enzyme inhibitors, anticholinergics, amantadine, and/or other drugs); (iii) symptoms and side effects (e.g., on and off times, dose failures, delayed time to on, tremor, dystonia, akinesia, bradykinesia, dyskinesia, tremor, nausea, vomiting, confusion, somnolence, hallucination, insomnia, constipation, dizziness, dysphagia, moods and mood changes, and impulse control disorders); (iv) sensor readings or data; (v) sleep times and/or sleep quality; (vi) meals and meal information, particularly of the protein content of the meal; (vii) defecation information; (viii) and/or exercise information. Such event markers can also record the time of the event and additional information or notes specific to each event, such as its intensity, quality, duration, amount, or character, among other information.

The pump may be programmed to increase the amount of drug infused following meals that contain proteins, after which the blood concentration of neutral amino acids competing with LD for active transport across the blood brain barrier increases.

The pump may be used to infuse the LD prodrug over the entire 24 hour day. Alternatively, in order to reduce the possibility of side effects (e.g., hallucinations) from 24 hour infusion of LD, some physicians may prefer that the pump only infuse the LD prodrug about 12, 14, 16, 18, or 20 hours per day. When the subject goes to bed at night, the infusion may be stopped or reduced significantly, i.e., reduced to less than 50% of the average daytime infusion rate. When the subject wakes up, he or she may initiate the infusion at the regular basal rate or, if the subject is in the off state, at a higher “morning dose” rate, in order to turn on more quickly. The pump can be programmed to begin such morning infusions automatically so that the subject does not need to initiate them. For example, the pump may be programmed to initiate infusion at the regular basal rate or at a higher morning dose rate at a certain hour or a certain amount of time (e.g., 4, 6, or 8 hours) after the infusion was stopped or decreased. If the pump is programmed to initiate such an infusion before the subject typically gets up in the morning, then the subject can get up in the on state rather than in the off state. A morning dose rate is an infusion rate that is greater than 10%, 20%, 30%, 40% or 50% greater than the basal rate or the average daytime infusion rate. When a fixed flow rate pump is used the subject may take an oral morning dose to turn on more quickly.

It may be difficult for a person with PD to input information or commands into the pump due to tremor or dyskinesia. Multistep inputs and those requiring fine motor skills (e.g., navigating through multiple menus on a screen, or using a keypad or a thumbwheel) may be particularly difficult. Consequently, a particularly useful means of providing input to the pump is to have one, two, three, four or more large, dedicated actuators on the pump or a remote control for the subject to easily activate in order to input frequently used or critical functions or information. Examples of such an actuator are one, two, three, four or more large buttons or switches that may be placed on the exterior of the pump or remote control. These buttons or switches may be of any convenient size. Examples include the range of 0.1 to 2.0 inches, the range of 0.25 to 1 inch. Examples of frequently used or critical functions or information may include: deliver bolus; reduce infusion rate; increase infusion rate; or experiencing one or more of dyskinesia, bradykinesia, tremor, off state or on state. Specific examples are: a button to indicate dyskinesia; a button to indicate bradykinesia; a button to indicate rigidity; a button to indicate off state; a button to indicate akinesia; or a button to initiate a bolus. When a fixed flow rate skin-adhered pump is used the flow would start e.g., upon its application to the skin.

The pump can be integrated with a sensor to form a sensor-augmented pump. The pump system can include the software, memory and hardware to enable the pump to input, store, recall, display, communicate and/or analyze sensor data useful to management of PD. Such integration may be physical, in which case the sensor and the pump share some physical components (e.g., a housing, remote control, memory, a display, a power source). Alternatively, such integration may be through data communication in which case the sensor transmits data to the pump, the pump transmits data to the sensor, or both. The sensor can include a transmitter and/or a receiver. The sensor can be a unitary device or may be a system having physically separate components, such as a physically separate sensor component and a display, memory, data communication, analysis or other component. The sensor can be reusable or disposable.

Sensors of the present invention can include any physiological, physical or chemical parameter associated with the subject. Specific examples of sensors and sensed parameters include: (i) motion sensors (e.g., accelerometers to sense movement, stillness, slowness, falling, walking, akinesia, bradykinesia, tremor, restless leg, finger movement and/or leg movement; (ii) the accelerometers may also sense posture, such as whether the subject is standing, sitting or lying down); (iii) pressure transducers or electrodes to sense cardiovascular parameters (e.g., heart rate, electrocardiogram, etc.); (iv) electrodes to sense wakefulness or sleep, and sleep parameters (these may include polysomnography, electroencephalogram, electro-oculogram, and/or electromyogram); (v) pressure sensors to measure blood pressure; (vi) acoustical or electrical sensors to detect snoring and/or sleep apnea; (vii) chemical sensors to test blood, saliva or other body fluids for the presence or concentration of specific medications or analytes (e.g., LD, other PD medications, coumadin, glucose, etc.); (viii) and a sensor to detect the subject's location, for example using input from a global positioning system or local computer or cell phone networks. An example of an accelerometer that can be used in the pump systems of the invention is the Chronos eZ430 wireless watch sold by Texas Instruments.

The pump system can include hardware, software and algorithms that enable the system to recognize a situation and recommend to the subject a one-time adjustment to the drug delivery regimen, e.g., to take a bolus of LD prodrug (e.g., LDA, LDE, LDC or LDS) optionally combined with a carbidopa prodrug (e.g., carbidopa ester or carbidopa amide). The pump system can include hardware, software and algorithms that enable the system to recognize patterns and recommend to the subject changes in his drug delivery regimen. The system can utilize for this purpose data from the stored event markers and data from sensors. The changes may be to the regimen of the drug being infused by the pump or to the regimen of other PD drugs being taken by the subject. For example: (i) if the system determines from user or sensor input that a subject has gone to bed or gone to sleep in the evening it may decrease the LD prodrug (e.g., LDA, LDE, LDC or LDS) infusion rate or stop the infusion altogether; (ii) if the system determines from user or sensor input that a subject has gotten out of bed or woken up in the morning it may provide a bolus of LD prodrug (e.g., LDA, LDE, LDC or LDS), increase the LD prodrug (e.g., LDA, LDE, LDC or LDS) infusion rate, or if the pump infusion had been stopped it may turn the pump infusion back on; (iii) if the subject has frequent or extended off periods then the system may recommend a revised drug infusion regimen with an increase in the LD prodrug (e.g., LDA, LDE, LDC or LDS) basal infusion rate; (iv) if the subject takes a long time to turn on after being off then the system may recommend a revised drug infusion regimen with an increase in the LD prodrug (e.g., LDA, LDE, LDC or LDS) bolus amount; (v) if the subject suffers dyskinesia, nausea or hallucination the system may recommend a revised drug infusion regimen with a decrease in the LD prodrug (e.g., LDA, LDE, LDC or LDS) basal infusion rate; (vi) if the subject suffers dyskinesia, nausea or hallucination the system may recommend that a scheduled LD prodrug (e.g., LDA, LDE, LDC or LDS) bolus be skipped or reduced; (vii) if user or sensor input indicates that the subject is suffering from akinesia the system may recommend that a one-time bolus be provided; (viii) if user or sensor input identifies a tremor the system may recommend that a one-time bolus of LD prodrug (e.g., LDA, LDE, LDC or LDS) be provided; and/or (ix) if the system determines that the subject consistently has a tremor at a certain time of day it may recommend a revised drug infusion regimen with an increase in the LD prodrug (e.g., LDA, LDE, LDC or LDS) infusion rate at that time of day.

The system may be programmed to recommend a one-time increase or decrease in the LD prodrug (e.g., LDA, LDE, LDC or LDS) basal infusion rate, a one-time bolus, or that a subject should skip a scheduled bolus. The system may also recommend a change to the LD prodrug (e.g., LDA, LDE, LDC or LDS) infusion regimen, such as increasing or decreasing the LD prodrug (e.g., LDA, LDE, LDC or LDS) basal infusion rate, increasing or decreasing the amount of a scheduled bolus, adding a new scheduled bolus, deleting a scheduled bolus, or changing the time of a scheduled bolus.

The system may also be programmed to similarly provide for one time increases or decreases, or to change the drug intake regimen, for other PD drugs that are being taken by the subject based on analysis of the event markers and/or input from sensors.

It will be appreciated that the pump system can be programmed to make some or all of these changes automatically, instead of simply recommending the changes to the subject.

The system may also be programmed to adjust the flow rate in order to maintain a steady LD influx in the CNS following a protein-rich meal, and thus avoid the symptoms of low brain LD, such as turning off. For example, LDEE is relatively rapidly hydrolyzed in vivo by abundant esterases. The transport to the brain is active transport, involving neutral amino acid transporters. The LD in the plasma competes with other neutral amino acids in the plasma for transport across the blood-brain barrier. The concentrations of the other neutral amino acids in plasma increase following a protein-containing meal, often reaching their peak 3-5 hours after the meal. It is therefore advantageous to gradually increase in the infused dose rate starting about 1 hour after a protein meal to reach a maximal dose rate at 3-5 hours after the meal, then decrease it, in absence of a second protein-rich meal, to base rate over about 2 hours. Thus, to maintain a steady LD influx in the CNS, the infusion rate can be adjusted to peak at about 1.7× the base rate following consumption of a protein-rich meal.

The system may also be programmed to adjust the flow rate to accommodate the user's sleep pattern. For example, if the user prefers not to use the infusion pump while asleep, the user can start the awake period with a higher than basal infusion rate (i.e., a bolus), optionally delivered over 10-60 minutes. The system may include a diurnal program that is user specific, varied for different users to account for the times of the day when they have meals, the protein-content of the individual meals, and their sleep/awake hours.

Containers (e.g., Cartridges and Vials)

Numerous approaches are available to storing and combining the formulation components in order to achieve drug stability and convenience.

The drug product or its components (e.g., a LDEE prodrug solution, a LDEE.HCl solution, its neutralizing base, diluents, preservatives, anti-oxidants, viscosity modifiers, and/or solutions of co-administered drugs like carbidopa prodrugs) may be stored in one, two, three, four or more containers. The containers may be physically separate or they may be physically connected, e.g., separate chambers in a common housing. One or more of the containers may be configured to be connected to the infusion pump. The containers or chambers may be configured so that their contents are manually combined by the user, or so that they are automatically combined by the infusion pump. For example, a metal foil or a plastic barrier separating the two chambers may be pierced or crushed when an actuator is pressed; the actuator may be automatically pressed when the container is inserted into the infusion pump. The contents of the containers may be combined outside the pump and then transferred to the pump's drug reservoir. Alternatively, one of the containers or chambers may serve as the pump's drug reservoir. The containers may be disposable or reusable. Exemplary forms of the containers are vials and syringes.

In one preferred embodiment, the storage container includes two or more sealed chambers, each chamber including a precursor solution of an infusible LD prodrug pharmaceutical composition. One chamber includes an acidic LD prodrug or LD prodrug and carbidopa prodrug solution. A second chamber includes a solution with a basic pH. Optionally, the storage container may include a means for combining or mixing the two or more solutions to form the infusible LD prodrug pharmaceutical composition. Examples of such a storage container are a multi-chamber syringe, and a multi-chamber drug reservoir of an infusion pump.

In a second preferred embodiment, the storage container includes two or more sealed chambers, the first chamber including the solid LD prodrug and optionally the carbidopa prodrug. The second chamber includes a solution of two acids, one being preferably HCl and the second being a polybasic acid, such as phosphoric acid. Optionally, the storage container may include a means for combining or mixing the two or more solutions to form the infusible LD prodrug pharmaceutical composition. Examples of such a storage container are a multi-chamber syringe, and a multi-chamber drug reservoir of an infusion pump.

The container or chamber may contain the LD prodrug (e.g., LDA, LDE, LDC or LDS) in liquid form or in dry solid form. It may also contain the carbidopa prodrug, e.g., its ester or amide.

When the LD-prodrug and/or carbidopa-prodrug are dissolved, the container or chamber is preferably impermeable to oxygen, e.g., constructed of glass; a non-porous ceramic; a relatively water vapor and oxygen impermeable polymer, such as polyacrylonitrile, polyvinylidene chloride, or filler loaded butyl rubber (poly(isobutylene-co-isoprene)); filler loaded chlorobutyl rubber; chlorobutyl rubber, bromobutyl rubber, butyl rubber, chlorosulfonated polyethylene (Hypalon), or amorphous polyethylene terephthalate; and metalized polymers (e.g., metalized polypropylene or polyester)). Typically the container or chamber has a wall thickness of from about 0.25 mm to about 1.5 mm (e.g., 0.25 to 0.5, 0.5 to 1.0, or 1.0 to 1.5 mm).

Materials may be selected for their compatibility with the formulation components (e.g., glycerol does not attack plastics, and polymers can be selected for their compatibility with water, alcohol, and mixed solvent systems). For example, polymers that do not increase their weight by more than 5% when soaked for 24 hours in the formulation components at 25° C. would be deemed compatible.

The container or chamber may include a vial made of glass, preferably of colored glass absorbing light of wavelengths shorter than about 450 nm. The vial may include a septum, made of a rubber, preferably inorganic filler loaded rubber, in which the permeability of oxygen is low, such as butyl rubber (poly(isobutylene-co-isoprene)); or chlorobutyl rubber or bromobutyl rubber.

The container may be hard-sided or flexible, such as a polymeric bag. The LD prodrug (e.g., LDA, LDE, LDC or LDS) can be placed into the container or chamber in such a manner that the contents of the container or chamber are substantially free of water and optionally, but not necessarily, also of oxygen. Methods of accomplishing this are well known in the art. They may include storing the composition under an inert gas. Alternatively, they may include using a vacuum to remove most gases from the container prior to or after pumping or injecting the dry solid LD ester into the container, and then sealing the container.

The containers of the invention can include a connector for connection to an ambulatory infusion pump. The connector can be as simple as a septum, which is punctured to place the container in fluid communication with the pump cannula. More complex male-female components for establishing the connection can be used to achieve the same purpose and are well known in the art.

The container can include multiple, individually sealed chambers containing the LD prodrug (e.g., LDA, LDE, LDC or LDS) formulation or its solid and/or liquid components and/or the carbidopa prodrug. Individual chambers may be opened and, if necessary, combined to provide the infused formulation. For example, two, three, four, five or more separate chambers containing dry solid LD prodrug (e.g., LDA, LDE, LDC or LDS) formulation may be employed. It can also include multiple chambers containing aqueous solvent. Such an approach permits the drug from one chamber to be used for infusion, while the drug in the other chamber remains stable in its sealed chamber. As the infusion solution made from the drug in one chamber is approaching depletion, is depleted, or is nearing the end of its stable lifetime, the drug in another chamber may be used to create a fresh infusion solution. In this manner a single container can provide infusion solution for significantly longer than the stable lifetime of a single infusion solution. Similar manufacturing methods and methods of use taught herein may be used to make and use a container including multiple chambers containing dry solid or liquid LD prodrug and/or carbidopa prodrug.

Dry Solid Form

In one embodiment, the LD prodrug with or without the carbidopa prodrug is stored in dry solid form. The present invention includes a method of preparing the infusion solution for use. Prior to use the dry solid LD prodrug (e.g., LDA, LDE, LDC or LDS) formulation is mixed with water or with an aqueous solution, such as an HCl and polybasic acid including aqueous solution, to create the infusion solution. The LD prodrugs and optional carbidopa prodrugs can be rapidly hydrolyzed in the body, and can be stored in the solid prodrug form at 25° C. for 6 months, 12 months, 18 months, or 24 months. They form infusible solutions that can be stable at about 25° C. for at least 16 hours, 1 day, 2 days, 3 days, 4 days or 7 days.

The present invention includes a process for manufacturing a container or chamber containing the LD prodrug (e.g., LDA, LDE, LDC or LDS) formulation by placing the dry solid LD prodrug (e.g., LDA, LDE, LDC or LDS) formulation into the container. In a first embodiment, the container may include a material that is substantially oxygen and water vapor impermeable, eliminating substantially all of the water vapor and oxygen from the compartment, and the process may include sealing the container, and subsequently combining the dry LD prodrug (e.g., LDA, LDE, LDC or LDS) formulation with an aqueous solution to create an infusion solution. In a second embodiment the container of the solid prodrug is stored in a second desiccated container and the process may include combining the dry LD prodrug (e.g., LDA, LDE), optionally containing a carbidopa prodrug, with an aqueous solution to create an infusion solution. Typically, the aqueous solution includes HCl and a polybasic acid.

Optionally, the process may also include the step of adding water or an aqueous solution to a second, optional, chamber in the container and sealing the second chamber. Optionally, the water or the aqueous solution is substantially free of dissolved oxygen and the material of the second chamber is substantially impermeable to oxygen. Optionally, the manufacturing process includes the step of the subject, or his caregiver, adding aqueous solution to the dry solid LD prodrug (e.g., LDA, LDE, LDC or LDS) formulation. The step of adding the aqueous solution may include combining the dry solid LD ester with an aqueous, for example HCl and polybasic acid including, solution stored in a second chamber of the container, or it may include adding the aqueous solution to the container from a separate source.

For the user of the solid prodrug its rapid dissolution is advantageous. Because the concentrations of the subcutaneously infused prodrugs are in the range between about 0.25 M and about 1.5 M, e.g., between 0.3 M and 1.0 M, or between 0.4 M and 0.8 M, or between 0.4 M and 0.6 M the dissolution may require several minutes. To accelerate the dissolution, the prodrug particles would require a high surface-to-volume ratio, in which case the mole % of surface adsorbed-water, not removed under acceptable drying conditions, could be high. The adsorbed water could hydrolyze the LDE or LDA or LDC or carbidopa ester or carbidopa amide upon its extended storage. Resolving the conflict between fast dissolution and water content, in a particular group of embodiments of this approach the solid stored in one container or chamber may contain LDE or LDA or carbidopa prodrug crystallites, their amines or hydrazines mostly or completely un-protonated, i.e, not their salt form. The large basic crystallites would be, generally, advantageously less hygroscopic than the salts formed of the protonated LDE or LDA cation and the chloride, bisulfate or sulfate anion. The chamber containing the LDE or LDA (with or without the carbidopa ester or amide) may optionally also contain a buffer-forming base, such as trisodium citrate or trisodium phosphate, in a molar amount typically less than 2 mole %, 1 mole % of the LDE or LDC. The second chamber would contain an about equivalent amount of the salt-forming acid solution, such as the hydrochloric acid solution or a slight excess of the acid, typically of about 1% of the equivalent amount or less. The stored basic LDE or LDC with or without the carbidopa ester or amide in one chamber and would be neutralized mostly by acid in the second chamber, e.g. 0.25 M-1.5 M HCl with typically 0.005 M-0.15 M of polybasicacid, e.g., about 0.3-0.8 M HCl, 0.01-0.08 M polybasic acid, or 0.4-0.8 M HCl, 0.01-0.06 M polybasic acid. Upon adding the acid to the solid base, it can dissolve in 5 minutes or less.

The LD prodrug (e.g., LDA, LDE, LDC or LDS or respective salt) with or without the carbidopa ester or amide solid dosage form can include one or more of the following: (i) a polycarboxylic acid (with the number of carboxylic acid functions exceeding the number of amines of the LD prodrug (i.e., LDA, LDC) and when a carbidopa prodrug is added the number of LD amines plus the number of carbidopa prodrug hydrazines. The environment of the LD prodrug molecules is thereby made acidic. In the acid environment, the catechol functions of the LD prodrug (e.g., LDA, LDE, LDC or LDS) or carbidopa prodrug molecules are less prone to oxidation, and the prodrugs are less prone to hydrolysis; (ii) a viscosity enhancing agent, which may also inhibit crystallization resulting in precipitation of large particles, in an amount such that, reconstituted the infusible formulation has a viscosity of between about 1.2 cp and about 10² cp at about 25° C.; (iii) a physiologically acceptable antioxidant (e.g., bisulfite, ascorbic acid (such as sodium ascorbate), p-aminophenol, acetamol, a t-butyl ortho-substituted phenol, or any antioxidant described herein); (iv) a physiologically acceptable crystal growth inhibitor (e.g., a polycarboxylic acid, collagen, albumin, polyethylene glycol, hydroxyethyl starch, dextran, glucose, glycerol, or mannitol); and (v) an enzyme inhibitor or agonist, such a DDC inhibitor or its prodrug like a carbidopa ester or amide, MAO-B agonist, and/or COMT inhibitor.

The solid dosage form can be packaged, for example, in a container (e.g., in a cartridge designed for insertion into an infusion pump, or a vial, the contents of which may be transferred to an infusion pump) of the invention for use in an infusion pump of the invention.

Aqueous Liquid Form

In a preferred embodiment, the LD prodrug is stored in liquid form, which may be aqueous. In one approach, a concentrated, i.e., >0.3 M, >0.5 M, >0.65 M, >1.0 M, >1.5 M, >2.0 M, >2.5 M aqueous liquid LD prodrug formulation, such as an LDE or LDC including formulation, exemplified by a solution including LDEE.HCl, is stored in a first container or chamber without substantial LD precipitation for >3 months, >6 months, >12 months, >18 months, >24 months, >36 months, or >48 months. The stored concentrated solution is acidic, of about pH 1.0-2.0, pH 2.0-3.0 (e.g., about pH 2.3), or pH 3.0-4.0, or pH 4.0-5.0. The preferred pH of the stored solution is 2.5±0.5. The concentration of an exemplary LDEE.HCl solution is 0.3 M to 0.35 M; 0.35 M to 0.45 M; 0.45 M to 0.55 M; 0.55 M to 0.65 M; 0.65 M to 0.75 M; 0.75 M to 1.0 M; 1.0 M to 2.0 M; 2.0 M to 3.0 M, 3.0 M to 3.5 M, or greater than 3.5 M. To this solution, a carbidopa prodrug, such as carbidopa ethyl ester hydrochloride may be optionally added in a molar amount of between about 10% and about 40% of the molar amount of the LDEE.HCl. The preferred molar amount of the carbidopa prodrug can be about 15% and 30% of the molar amount of LDEE.HCl, for example ¼ of the molar amount LDEE.HCl. The first container or chamber can be impermeable to oxygen and may include the materials previously identified in this application. A second container or chamber contains a basic solution, such as a concentrated solution of a base, optionally forming a buffer. While simple bases like sodium hydroxide or potassium hydroxide may be used, the preferred bases include a pharmaceutically acceptable potassium and/or a sodium salt of a dibasic, tribasic or tetrabasic acid. Exemplary salts include those of citric acid; pyrophosphoric acid; succinic acid or phosphoric acid, like trisodium citrate, tetrasodium pyrophosphate, disodium succinate or trisodium phosphate. Prior to use, enough of the solution in the second container is transferred to, or otherwise combined with, that in the first container to increase the pH e.g., from about 2.5±0.5 to about pH 4.8±0.8, for example to pH 4.2±0.3 or pH 5.0±0.5. When the concentration of the base, e.g., trisodium citrate, is for example about 1 M or greater, the volume of the basic solution added to increase the pH can be between 10⁻²- and 0.1 mL per mL of the exemplary LDEE.HCl solution; when its concentration is 0.1 M, between 0.1 mL and 1 mL may be added per mL of the exemplary LDEE.HCl solution. When its concentration is 0.02 M, between 0.5 mL and 5 mL may be added per mL of the exemplary LDEE.HCl solution.

The present invention includes a process for manufacturing a container containing the LD prodrug (e.g., LDE or LDC) formulation by placing the solution of the LD prodrug (e.g., LDE or LDC) formulation into a container or chamber, the container or chamber including material that is substantially oxygen impermeable, eliminating substantially all of the water vapor and oxygen from the container or chamber, and sealing the container or chamber. Optionally, the manufacturing process includes the step combining the aqueous LDE or LDC solution with a basic solution, optionally stored in a second chamber of the container.

Alternatively, the aqueous liquid formulation is an oil-in-water emulsion, where the prodrug is in the oil phase. When the liquid formulation is an emulsion (e.g., includes a lipid and/or an alcohol (e.g., glycerol)), an LDE and/or LDA including formulation can be stored in a container of the type taught hereinabove.

The LD prodrug (e.g., LDA, LDE, LDC or LDS or their respective salt) aqueous liquid dosage form can include one or more of the following (i) a physiologically acceptable buffer (e.g., disodium succinate or trisodium citrate); (ii) a physiologically acceptable antioxidant (e.g., bisulfite, a salt of ascorbic acid (such as sodium ascorbate), p-aminophenol, acetamol, a t-butyl ortho-substituted phenol, or any antioxidant described herein); (iii) a physiologically acceptable crystal growth inhibitor (e.g., a polycarboxylic acid, collagen, albumin, polyethylene glycol, hydroxyethyl starch, dextran, glucose, glycerol, or mannitol); (iv) a viscosity enhancing agent in an amount such that, reconstituted the infusible formulation has a viscosity of between about 1.2 cp and about 10² cp at about 25° C.; and (v) an enzyme inhibitor or agonist, such a DDC inhibitor, exemplified by the carbidopa prodrugs e.g., carbidopa ester or carbidopa amide, MAO-B agonist, and/or COMT inhibitor.

The LD prodrug can be dissolved in a lipid or in an emulsion-forming, preferably oil-in-water emulsion-forming, mixture prior to infusion. The present invention includes a method of preparing the infusion solution for use. The lipid and/or alcohol or oil-in-water emulsion including liquid may be stored in an oxygen-impermeable container or chamber, as taught hereinabove.

The invention includes a method of preparing the lipid and/or alcohol-based or emulsion-based infusion solution for use, as well as a process for manufacturing a container containing the lipid and/or alcohol or emulsion based LD prodrug formulation by placing the lipid and/or alcohol-dissolved or emulsified LD prodrug formulation into a container or chamber, the container or chamber including material that is substantially oxygen impermeable, eliminating most of the oxygen from the container or chamber and sealing the container or chamber. Alternatively, the lipid and/or alcohol or the emulsion forming mixture may be added to the LD prodrug containing container or chamber prior to use.

The invention also features a disposable, optionally skin adhered drug container including a pharmaceutical composition of the invention. In particular embodiments the container, or a chamber of the container, includes an inert atmosphere, is substantially free of water, or substantially free of oxygen.

The formulations of the invention are placed into an infusion pump drug reservoir prior to use or may come pre-loaded in a pump reservoir. Reservoir volumes are typically equal to or less than 1, 2, 3, 4, 5, 7.5, 10, 12.5, 15, 17.5, or 20 mL. The reservoir may be reusable or disposable.

The liquid dosage form can be packaged, for example, in a container of the invention for use in an infusion pump of the invention, or can be prepared just prior to infusion.

Non-Aqueous Liquid Compositions and Emulsions

The dosage forms of the invention can be liquid dosage forms or solid dosage forms, which can be reconstituted in a lipid and/or alcohol solution. The liquid dosage form can be a non-aqueous solution containing a liquid selected from ethanol, sesame oil, castor oil, cottonseed oil, benzyl benzoate, or a mixture thereof, or an emulsion. Typically the no-aqueous compositions are infused intragastrically, intraduodenally or intrajejunally.

The formulations of the invention are placed into a container or an infusion pump drug reservoir prior to use or may come pre-loaded in a reservoir. Reservoir volumes are typically equal to or less than 1, 2, 3, 4, 5, 7.5, 10, 12.5, 15, 17.5, or 20 mL. The reservoir may be reusable or disposable. In one embodiment the formulation is both stored and used in solution form. In a second embodiment the formulation is stored as a solid and is combined with the lipid and/or alcohol-based excipient, typically a mixture of lipids and/or alcohols, prior to use. In one embodiment the container includes two separate, sealed chambers. The first chamber contains the solid formulation in a substantially dry, optionally oxygen free environment. The second chamber contains the lipid, such as sesame oil, castor oil, or cottonseed oil and optionally water and an emulsifier. The contents of the two chambers may be combined immediately prior to use either by the user or by a mechanism in the infusion pump itself. For example, a metal foil and/or alcohol or plastic barrier separating the two chambers may be pierced or crushed when an actuator is pressed in the container. The actuator may be automatically pressed when the container is inserted into the infusion pump.

Some of the excipients used for the solid dosage forms can likewise be included in the liquid dosage forms of the invention. For example, certain lipid and/or alcohol-soluble anti-oxidants, such as vitamin E or tert-butyl substituted phenols may be co-dissolved in the lipid, e.g., the oil.

The melting points and the liquidus temperatures of mixtures are lower than those of at least some of their pure components. Mixtures of lipids (e.g., those including triglycerides that are crystalline solids at ambient temperatures) are often liquids at ambient temperatures. For example, the solidus temperature of palm or coconut oil is 20-24° C. At this temperature the oil starts scattering light because of formation of a solid phase. The major constituent (44 weight %) of palm oil is the triglyceride glycerol tripalmitate melting at 65° C.; its second most abundant constituent (38 weight %) is glycerol trioleate, melting at −4° C. One may say that the glycerol tripalmitate dissolves in the glycerol trioleate; alternatively one may say that the glycerol trioleate suppresses the melting point of the glycerol tripalmitate. LD prodrugs and carbidopa prodrugs can also be soluble in alcohols like glycerol, ethylene glycol, propylene glycol, and ethanol, which can be used as solvents or co-solvents. The preferred alcohol solvent is glycerol.

The lipids serve in general to lower the liquidus temperature of the mixture including the LD prodrug, the combined concentrations of which is at least 0.65 moles per liter. The added lipid or lipids lower the liquidus temperature to below about 30° C., preferably to below about 20° C. and most preferably to below about 15° C. The lipids may include, for example, triglycerides of carboxylic acids. The preferred carboxylic acids of the triglycerides have an even number of carbon atoms. The number of carbon atoms is typically between 2 and 22; it is preferably between 12 and 20, e.g. 12 and/or 14 and/or 16 and/or 18 and/or 20. The triglycerides can be saturated, mono-unsaturated or polyunsaturated. Mono and polyunsaturated triglycerides are preferred, as are unsaturated oils with cis double bonds in their carboxylic acids, preferred over those having trans double bonds. The lipids may also include a low-melting cholesterol ester, such as cholesterol arachidonate, ricinoleate, linoleate, palmitoleate, and/or oleate. Typically the viscosity of the lipids is greater than about 1.2 cP, usually it is greater than about 20 cP. While at higher viscosity the power consumption for pumping is greater, the rate of oxidation of the LD prodrugs, and when added, also of the carbidopa prodrugs by dissolved oxygen is reduced.

An LD prodrug from a single container (e.g., a cartridge or vial) may be infused by the pump, e.g., intragastrically, intraduodenally or intrajejunally, optionally through a nasogastric, nasoduodenal, or nasojejunal tube of less than about 4 mm, 3 mm, 2 mm, 1.5 mm, 1.0 outer diameter, and/or an internal diameter of less than 1 mm, 0.7 mm, 0.35 mm for a period of greater than or equal to about 8 hours, 12 hours, 24 hours, 48 hours, 72 hours and most preferably 96 hours. The container may contain between about 1 millimole and about 60 millimoles of LDE, LDC, and/or LDA. It may optionally additionally contain between about 0.2 millimoles and about 24 millimoles of carbidopa prodrug. These values correspond, in the exemplary case of LDEE, to about 225 mg and about 13.5 g of the compound. Preferably the container may contain between about 1 g and about 5 g of the LDEE and, if added, between about 0.2 g and about 1 g carbidopa ethyl ester. For an exemplary 2 M lipid and/or alcohol based solution, the corresponding volume contained in the container is between about 2.2 mL and about 11 mL.

The liquid dosage form of the invention can be a lipid solution, such as a solution including sesame oil, or castor oil, or cottonseed oil. It may also include an alcohol like glycerol or ethanol to modify the viscosity and/or to decrease the liquidus temperature.

Therapy

The formulations can be administered to subjects in therapeutically effective amounts. For example, an amount is administered which prevents, delays, reduces, or eliminates the symptoms of PD. Typical infused dose ranges are from about 20 μmole/kg to about 140 μmole/kg of LD prodrug (e.g., LDA, LDE, LDC or LDS or a salt thereof), per day. The typical daily dose of the optionally co-infused carbidopa prodrug is between about 5 μmole/kg and about 35 μmole/kg. For example, the typical daily dose for a subject weighing 75 kg is from about 1.5 millimoles to about 10 millimoles of LD prodrug (e.g., LDA, LDE, LDC or LDS or a salt thereof). The exemplary dosage of LD prodrug (e.g., LDA, LDE, LDC or LDS) to be administered is likely to depend on such variables as the stage of the PD patient (e.g., the dose/kg being higher for patients in more advanced stages of the disease), and the particular formulation of LD prodrug (e.g., LDA, LDE, LDC or LDS) being used. Optionally, a molar amount of a carbidopa prodrug between about 10% and about 40% of the molar amount of the LD prodrug, for example between 15% and 30%, may be added.

In order to avoid a local rise in the decarboxylation, de-amination or transamination product near the infused site that can cause local swelling, inflammation, erythema or nodule formation or other local adverse effects an enzyme inhibitor or agonist, such a DDC inhibitor, e.g., carbidopa, or carbidopa prodrug, a MAO-B agonist, and/or a COMT inhibitor can be co-infused in a systemically sub-therapeutic amount. The molar amount of co-infused carbidopa, carbidopa prodrug, MAO-B agonist, and/or COMT inhibitor can be between 0.1% and 10% of the molar amount of the infused LD-prodrug. For the typically infused LD-prodrug dose range from about 20 mole/kg to about 140 mole/kg, the dose range of the co-infused enzyme inhibitor or agonist can be between about 20 picomole/kg and about 14 mole/kg. For example, for local DDC inhibition the typical daily dose of the optionally co-infused carbidopa or carbidopa prodrug in a subject weighing about 75 kg can be between about 1.5 mole and about 1 millimole.

Modes of delivery of the aqueous formulations are via fixed flow rate or programmed infusion, preferably continuous infusion, and for formulations in which the prodrug concentration is between 0.25M and 1.5 M, e.g., between 0.25 M and 0.8 M, or 0.4 M and 0.6 M most preferably by continuous, subcutaneous infusion. The preferred route of delivery of the higher concentration or non-aqueous formulations is intra-gastric, intraduodenal or intra-jejunal, e.g., via a tube, which can be optionally a nasogastric, nasoduodenal or nasojejunal tube, of less than 4 mm, 3 mm, 2 mm, 1.5 mm, 1.0 mm outer diameter.

The LD prodrug concentration range in the subcutaneously infused solution is between 0.25 M and 1.5 M. At lesser concentrations than about 0.25 M the daily subcutaneously infused volume in a patient requiring daily 5 millimoles of LD or of the prodrug may exceed 20 mL and may cause edema or excessive swelling. Subcutaneous infusion of a solution of a concentration greater than about 1.5 M can cause the formation of subcutaneous nodules. The preferred concentration of the LD prodrug in the subcutaneously infused solution can be between 0.3 M and 1.0 M, more preferably 0.3 M and 0.8 M, for example, 0.4±0.1 M, 0.5±0.1 M, 0.6±0.1 M or 0.7±0.1 M, or 0.8±0.1 M. The pH of the infused solution is typically between 4.0 and 6.0, for example, 4.0±0.5, 4.5±0.5, or 5.0±0.5. The infused solution is typically stable, meaning clear and free of precipitated LD, for at least about 8 hrs at about 37° C., for example for at least about 16 hrs, 24 hrs, or 48 hrs.

Potential adverse effects can be ameliorated by infusing LD prodrug (e.g., LDA, LDE, LDC or LDS) in combination with an orally taken or co-infused enzyme inhibitor or agonist, such a DDC inhibitor, e.g., a carbidopa prodrug, MAO-B agonist, and/or COMT inhibitor; and/or anti-emetic agent, such as nicotine, lobeline sulfate, pipamazine, oxypendyl hydrochloride, ondansetron, buclizine hydrochloride, cyclizine hydrochloride, dimenhydrinate, scopolamine, metopimazine, or diphenidol hydrochloride. In certain instances it may be desirable to incorporate the anti-emetic into the formulation for simultaneous infusion in combination with the LD prodrug (e.g., LDA, LDE, LDC or LDS).

Preferred Sites and Depths of the Infusion

The preferred route of administration of the aqueous formulations is subcutaneous infusion with a cannula or two or more cannulae, and/or with a needle or two or more needles, preferably infusion below the dermis. Typical depths below the surface of the skin where the solutions may be infused are between about 4 mm and about 15 mm, the preferred depth being between about 5 mm and about 11 mm. In order to accelerate the dispersion of the infused solution from the tip of the inserted cannula or needle, flow-retarding, e.g., dermal or connective tissue hyaluronic acid/hyaluronate can be locally, transiently hydrolyzed by hyaluronidase added to the infusate. Depolymerization of the hyaluronic acid/hyaluronate by hyaluronidase can accelerate the flux of the prodrug or the LD produced of the prodrug by the action of esterases to blood venules and through these to the circulatory system. The hyaluronidase can be optionally recombinant, i.e., human hyalorunidase-like, but bacterially produced.

Because the concentrations of the subcutaneously or intramuscularly infused LD prodrug solutions generally are >0.3 M, 0.4 M, >0.5 M, >0.65 M, >1.0 M, it is desired that the solution be rapidly diluted following its infusion. Rapid dilution reduces the likelihood and magnitude of unwanted side effects at or near the infused site or sites. It is preferred to infuse the aqueous LD prodrug solution subcutaneously or intramuscularly at sites where the tissue-fluid is not stagnant, i.e., it flows because of abundance of arterioles and venules and/or movement of voluntary muscles or involuntary muscles; and/or proximal to major lymphatic vessels. The distance from the infusion site at which the concentration of the infused solution is halved decreases with flow, meaning it increases with the residence time, which is the inverse of the volumetric flow-rate of the tissue's fluid. Table 2, below, shows the estimated distance from the infusing orifice over which the concentration drops to ½ of the initial when the diffusion coefficient is 3×10⁻⁶ cm²s⁻¹ and the infusion rate of 3 μL min⁻¹.

TABLE 2

| | Residence time, min | | | | | | | | | | |
|---|---|---|---|---|---|---|---|---|---|---|---|
| | 1 | 2 | 3 | 4 | 5 | 6 | 7 | 8 | 9 | 10 | ∞ |
| Distance, mm | 0.45 | 0.61 | 0.73 | 0.82 | 0.9 | 0.97 | 1.04 | 1.1 | 1.15 | 1.2 | 26.5 |

For a stagnant solution the distance from the orifice to points at which the concentration drops to ½ the initial is as long as 26.5 mm. Even slight flow reduces the distance. For a residence time as long as 10 min, the distance already drops to 1.2 mm. For a 1 min residence time it is as short as 450 μm. During daytime and near a large and frequently used muscle or near the diaphragm, the residence time is typically less than 4 min and the radius of the most affected zone is less than about 820 μm. The desired flow of the infused tissue-fluid, for example either the subcutaneous fluid or the intramuscular fluid, is effectively induced by movement of proximal large voluntary muscles that are exercised during periods in which the subject is awake. Examples of such large muscles include the trapezius, deltoid, pectoralis major, triceps brachii, biceps, gluteus maximus, sartorius, biceps femoris, rectus femoris, and gastrocnemius muscles. The desired flow of the infused subcutaneous tissue-fluid is also induced by movement of proximal large involuntary muscles exercised during periods in which the subject is either awake or asleep, such as the diaphragm. It is therefore preferred to infuse the concentrated LD prodrug solution subcutaneously near these muscles. Some preferred infusion zones, for example diaphragm-moved upper/central abdominal zones, can be recognized by visible movement of the skin upon the movement of the proximal muscle, e.g., of the diaphragm upon inhalation or exhalation of air.

Multiple Point Infusion

Because concentrated and/or acidic subcutaneously infused drug solutions can damage cells near the tip of the infusing cannula or needle, it is advantageous to infuse through multiple orifices, i.e., cannulae and/or needles. Their infusion orifices are spaced preferably at distances greater than about 1 cm, 2 cm, 3 cm, 5 cm, 10 cm, 15 cm, 20 cm or 30 cm. A skin-adhered elongated strip, of a length to width ratio of 2 or more, with two cannulae or needles typically separated by more than 1 cm, 2 cm, 3 cm, 5 cm or 10 cm can be advantageously used.

Multiple point infusion can be carried out by a pump driving the fluid in multiple tubings, and/or cannulae, and/or needles; and/or by multiple pumps, each pump driving the fluid in one or more tubing and/or cannula and/or needle. The infusion can be through 2 or more, 4 or more, 9 or more cannulae or needles, the tips of which may be horizontally and/or vertically separated.

Optionally, two drug pumps can be used for the subcutaneous infusion, one infusing, for example in the left arm, the second in the right arm or in the abdominal region. Multiple point infusion can be also carried out with a perforated plastic cannula having one or more orifices along its length. The orifices may have similar diameters or they may differ in their diameter, for example such that the flow through the orifices will be about the same. This can be accomplished, for example, by making orifices distal from the pump larger than orifices proximal to the pump.

The prodrug containing aqueous solution may be delivered alternatively with a skin patch including a microneedle array in the dermis, typically at a depth of between 1 mm and about 3 mm below the epidermis. Microneedle arrays for drug delivery are described, for example, in U.S. Pat. Nos. 6,256,533, 6,379,324, 6,689,100, 6,980,555, 6,931,277, 7,115,108, 7,530,968, 7,556,821, 7,914,480, 7,785,301, 7,658,728, and 7,588,552 and in U.S. Patent Publication No. 20080269666.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention. The pH values refer, unless otherwise mentioned, to their values at the start of the experiments at about 23±2° C.

### Example 1

### Preparation of LDEE

LDEE of >99.5% purity (as determined by HPLC) was prepared according to Scheme 1, in general as described in U.S. Pat. No. 5,354,885.

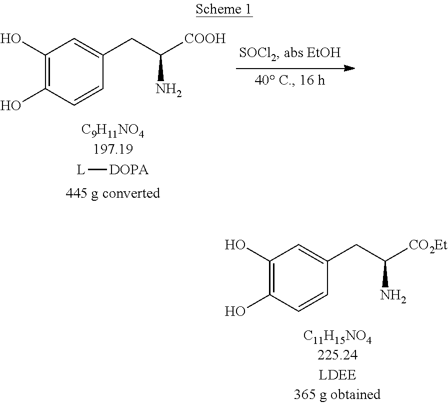

The LDEE was colorless, crystalline, melting in the temperature range of 84.5-86.5° C. and contained no HPLC-UV-vis detected L-DOPA. The hydrolysis of LDEE was monitored by HPLC (Agilent SB C18, 4.6 mm×150 mm, 3.5 μm; Mobile Phase A: H₂O/0.05% methanesulfonic acid, Mobile Phase B: 50% acetonitrile/50% H₂O/0.05% methanesulfonic acid; A:B 95%/5% (t=0 minutes), 95%/5% (t=3 minutes), 0%/100% (t=10 minutes), 0%/100% (t=14 minutes), 95%/5% (t=15 minutes), 95%/5% (t=20 minutes)). The observed retention time for LD was about 3.3 minutes and the observed retention time of LDEE was about 7.8 minutes.

### Example 2

### Precipitation of LD from a 0.25 M Physiological Saline Solution of LDEE Held at 37° C. For 16 Hours

150 mg of LDEE was dissolved in 3 mL of physiological saline (0.90% weight/volume of NaCl/water) at about 23° C., then at about 37° C. an additional amount of 22 mg of LDEE was added for a total LDEE concentration of about 57.3 mg/mL or about 0.25M. After holding the initially clear solution for 16 hours at 37° C. extensive precipitation of LD was observed.

### Example 3

### Precipitation of LD from a pH 6.75, 1.3 M LDEE/LDEE.HCl Solution Held at 37° C. For 16 Hours

At the ambient temperature of about 23° C., 226 mg of LDEE was dissolved in 1 mL of 1 M HCl. To the formed aqueous LDEE.HCl solution an additional amount of about 184 mg LDEE was added, followed by about 0.1 mL deionized water. The pH of the resulting clear, precipitate-free, solution was about 6.75 and its temperature was about 26.8° C. The estimated sum of the LDEE and LDEE.HCl concentrations was about 1.3 M. After the solution was held at 37° C. for 3 hours, there was no precipitation, but there was extensive precipitation of LD after 16 hours at 37° C.

### Example 4

### Precipitation of LD from a pH 6.87, 0.7 M LDEE/LDEE.HCl Solution Held at 37° C. For 16 Hours

At the ambient temperature of about 23° C., about 113 mg of LDEE was dissolved in 1 mL of 1 M HCl. To the formed aqueous LDEE.HCl solution an additional amount of about 87 mg LDEE was added, followed by about 0.1 mL distilled water. The temperature of the resulting clear, precipitate-free, solution was about 27.2° C. pH and its pH was about pH 6.87. The estimated total concentration of LDEE and LDEE.HCl was about 0.7 M. After the solution was held at 37° C. for 3 hours there was no precipitation, but there was extensive precipitation of LD after 16 hours at 37° C.

### Example 5

### Precipitation of LD from a pH 7.01, 1.2 M LDEE/LDEE Acetate Solution Held at 37° C. For 16 Hours

At the ambient temperature of about 23° C., about 226 mg of LDEE was dissolved in 1 mL of 1 M acetic acid. To the formed aqueous LDEE acetate salt solution an additional amount of about 168 mg LDEE was added. A white suspension was formed. After adding about 0.1 mL distilled water most, but not all, of the suspended particles dissolved. The pH of the resulting suspension was about 7.01 at about 26.4° C. The sum of the concentrations of LDEE and LDEE acetate was about 1.2 M. After being held at about 37° C. for 3 hours the suspension remained cloudy, but there was no heavy precipitation. There was, however, extensive precipitation of LD after 16 hours at 37° C.

### Example 6

### Preparation of a 2.4 M LDEE Acetate Solution and Rapid Hydrolysis of the LDEE Acetate at pH 4.7

The LDEE acetate salt, meaning the salt formed of LDEE and acetic acid, was prepared by adding under nitrogen 2.25 g (10 millimoles) LDEE in small portions to 2 mL of a magnetically stirred 5.0 M solution of acetic acid. The pH of the resulting colorless, clear (meaning precipitate free) solution was 5.3. After adding 0.175 mL glacial acetic acid, the pH was 4.7. The total volume was about 4.1 mL, i.e., the concentration of the acetate salt of LDEE was about 2.4 M. At this concentration the LDEE acetate equivalent molar amount of 1 g LD is dissolved in about 2.1 mL. HPLC of the solution showed that the initial % LD was between 0.22% and 0.25% meaning that the LDEE:LD ratio was about 99.8:0.2. After standing at the ambient temperature of about 23±2° C. for 24 h, 4.5% of the LDEE was hydrolyzed and after 96 hours 14.5% was hydrolyzed to LD. Nevertheless the solution was clear, i.e., precipitate free. The initial rate of hydrolysis was about 4.0±0.4% per day.

### Example 7

### Preparation of LDEE Hydrochloride (LDEE.HCl) and its High Solubility (2.5 M Solution) at pH 4.6 at Ambient Temperature

To 2 mL of aqueous 5.0 M HCl in a vial, solid, pure LDEE (2.25 g, 10 millimoles) were added in portions under nitrogen with magnetic stirring. After about 3 hours of stirring at ambient temperature of about 23±2° C. the solution was clear and the pH was 0.28. The pH was adjusted to 4.60 by adding 160 mg of sodium acetate. The volume was about 4 mL and the concentration of the LDEE.HCl solution was about 2.5 M. The experiment shows that LDEE.HCl in a molar amount equivalent to the molar amount of the typical daily dose of 1 g LD can be dissolved in about 2 mL of an aqueous solution.

### Example 8

### Precipitation of LD from 2.6 M LDEE.HCl at pH 5.5 after about 30 h

1.7 g of LDEE (7.5 millimoles) was dissolved in 1.405 mL of aqueous 5M HCl, then enough trisodium citrate was added to increase the pH to 5.5. LD precipitated after stirring at ambient temperature solution for about 30 hours.

### Example 9

### LD-Supersaturation in a 2.6 M LDEE.HCl Solution and Precipitation of LD after 7 Days of Storage at 23±2° C. at pH 5.1

An about 1 M trisodium citrate solution was prepared by dissolving in 1.353 g of trisodium citrate dihydrate in 4.0 mL water. 1.928 g (8.57 millimoles) of LDEE were dissolved in 1.35 mL of aqueous 6.0 M HCl, then 0.25 mL of the about 1.0 M citrate solution was added to form a pH 5.1 solution of about 3.2 mL volume, in which the concentration of LDEE and its salt(s) was about 2.65 M.

After flushing with nitrogen, the solution was kept for 7 days at 23±2° C. in a vial sealed with a grey elastomeric, substantially oxygen impermeable septum. After 7 days, 6.2% of the LDEE was hydrolyzed to LD. Although the corresponding LD concentration was about 35 g per liter, twice the concentration at saturation according to Example 16 (at pH 4.5 and at about the same temperature), precipitation of LD started only after small LD seed crystals were added. One hour after its seeding with LD crystals and stirring, LD precipitated. Because about 0.88% of the LDEE is hydrolyzed daily LD, precipitation from a constant pH 5.1 solution is expected only after about 4 days. An operational life of 4 days suffices for infusion.

### Example 10

### Rapid Hydrolysis of 2.5 M LDEE.HCl at pH 0.12

1.12 g of LDEE were dissolved in about 1 mL of aqueous 5 M HCl to produce an about 2.5 M LDEE.HCl solution of pH 0.12. After flushing with nitrogen, the solution was kept for 6 days at 23±2° C. in a vial sealed with a grey, oxygen impermeable septum. After 6 days at 23° C., about 4.3% of the LDEE.HCl was hydrolyzed, i.e., about 0.71% of the LDEE.HCl was converted daily to LD at pH 0.12. This shows that the hydrolysis is rapid not only at pH>4 but also at pH<1.0.

### Example 11

### Slow Hydrolysis of LDEE.HCl at pH 3.3 at 23±2° C.

An about 2.6 M LDEE.HCl solution was prepared of 1.5 g LDEE and 1.24 mL of aqueous 5 M HCl plus trisodium citrate and citric acid to adjust the pH to 3.3. After flushing with nitrogen, the solution was kept for 6 days at 23±2° C. in a vial sealed with a grey elastomeric, oxygen impermeable septum. The volume was about 2.5 mL. After 6 days at 23±2° C., 1.8% of the LDEE was hydrolyzed to LD (and ethanol and HCl which were not assayed). At this pH and temperature about 0.30% of the LDEE.HCl was hydrolyzed daily.

### Example 12

### After 24 Hours at pH 2.1 and at 23° C., No HPLC-Detected Hydrolysis of 2.6 M LDEE.HCl

An about 2.6 M LDEE.HCl pH 2.1 aqueous solution was prepared by dissolving 1.506 g LDEE in 1.3 mL of aqueous 5M HCl and adjusting the pH by adding 0.24 μL of 1 M HCl. After flushing with nitrogen, the solution was kept for 24 h at 23±2° C. in a vial sealed with an elastomeric grey, oxygen impermeable septum. After 24 hours, the pH was 2.0. HPLC-UV-vis assay of the solution did not detect LD in the solution.

### Example 13

### Estimate of the Operational Life of the pH 4.5 Citrate Buffered 2.5 M LDEE.HCl Solution at 23° C.

A solution of 294 mg of trisodium citrate dihydrate (1 millimole) in 0.864 mL of water was prepared; its weight was 1.136 g. 0.135 g this solution were added to the LDEE.HCl solution of Example 8 to produce a 2.5 M LDEE.HCl aqueous solution of pH 4.5. After flushing with nitrogen, the solution was kept in a vial sealed with a grey, elastomeric oxygen impermeable septum. After 24 hours at the ambient temperature of 23±2° C., only 0.39% of the LDEE.HCl was hydrolyzed to LD. The experiment, in combination with the solubilities of Example 16, suggests an operational life of about 9 days, meaning that the time to LD saturation leading to possible precipitation is about 9 days at about 23° C.

### Example 14

### Estimate of the Operational Life of the pH 4.5 Citrate Buffered 2.5 M LDEE.HCl Solution at 29-30° C.

The pH 4.5, 2.5 M LDEE.HCl aqueous solution was prepared as in Example 13. After flushing with nitrogen, the solution was kept in a vial sealed with a grey elastomeric, oxygen impermeable septum. After 4 days at 29° C.-30° C., 3.4% of the LDEE.HCl was hydrolyzed, showing that the solution has an operational life, meaning time to LD saturation that might lead to some LD precipitation, of 4 days, well in excess of the required operational life of 24 hours. Projection to 37° C. suggests that at body temperature the operational life is about 2 days. If the solution would be stored, for example, for one year at 4° C., where its storage (shelf) life is projected to be greater than 4 years (per Example 18), then warmed to the 23° C. ambient temperature, its residual operational life would still be longer than 4 days. If it were stored at 4° C. for 18 months, then warmed to its operational ambient temperature, the solution would still have a residual operational life of more than 3 days, sufficient for infusion.

### Example 15

### Hydrolysis of Acetate Buffered 2.5 M LDEE.HCl of pH 4.6 at 23° C.

To 2 mL of aqueous 5.0 M HCl in a vial, 2.25 g (10 millimoles) of LDEE was added in small portions under nitrogen with magnetic stirring at about 23±2° C. After about 3 hours the solution was clear and its pH was 0.28. Its pH was raised to 4.60 by adding 160 mg of sodium acetate. The volume was about 4 mL and the concentration of the LDEE.HCl solution was about 2.5 M. After flushing with nitrogen, the solution was kept in a vial sealed with a grey elastomeric, oxygen impermeable septum. HPLC assay showed that after 72 hours at about 23±2° C. about 2.4% of the LDEE.HCl was hydrolyzed to LD (plus ethanol and HCl which were not assayed), i.e., that 0.82% of the LDEE.HCl and/or LDEE acetate and/or LDEE was hydrolyzed per day. This is about ⅕^{th} of the daily rate of the acetate salt of Example 6. The five-fold slower rate than that of acetate shows that the hydrolysis rate is anion-dependent and is substantially slower for the chloride of LDEE than it is for its acetate.

### Example 16

### High Solubility of LD in Concentrated Aqueous LDEE.HCl Solutions at pH 4.5

Three solutions of a pH near 4.5 were prepared by adding 1 M trisodium citrate to increase the pH and 1 M HCl to decrease the pH. The about 1 M trisodium citrate solution was prepared by dissolving 1.353 g of trisodium citrate dihydrate in 4.0 g of water. The solubility of LDEE.HCl was determined at about 25±1° C. The first solution was prepared by dissolving 1.52 g LDEE in 1.3 mL 5 M HCl and adding 0.05 mL of about 1M citrate solution. The volume was about 2.5 mL and the LDEE.HCl concentration was about 2.7 M. A second solution was prepared by diluting the first solution with an equal volume of water, to form an about 1.35 M solution of LDEE.HCl. The third solution was made by adding to 0.6 mL of the about 1.0 M citrate solution 0.8 mL of 1.0 M HCl. The solubility of LD was measured in 1 mL samples of the three stirred solutions by adding sequentially small amounts of LD and watching their dissolution. In absence of LDEE.HCl, the solubility of LD was about 5.8 g per liter. In the 1.35 M LDEE.HCl solution it was about 10.2 g per liter; and in the 2.7 M LDEE.HCl solution it was as high as about 17 g per liter.

The experiments show that the solubility of LD increases with the concentration of LDEE.HCl. It is in 2.7 M LDEE.HCl about three times more soluble than it is in the absence of LDEE.HCl.

### Example 17

### Temperature Dependence of LDEE.HCl Hydrolysis at pH 2.3, 3.0, 4.0 and 5.0

An aqueous LDEE.HCl solution was prepared of 6.002 g of LDEE and 5.2 mL of aqueous 5.0M

HCl. An about 1 M trisodium citrate solution was prepared of 1.353 g of trisodium citrate dihydrate and 4.0 g of water. A pH 2.03 solution was prepared by adding 0.321 mL of 1.0 M HCl and 25 μL of the 1.0 M trisodium citrate solution. The volume was about 10.6 mL and the LDEE.HCl concentration was about 2.5 M. To about 9.8 mL of this solution 25 μL of a 1.0 M trisodium citrate solution were added to produce the pH 3.0 solution, having a concentration of LDEE.HCl of about 2.5 M. To about 8.2 mL of this solution 90 μL of a 1.0 M trisodium citrate solution were added to produce the pH 4 solution, the LDEE.HCl concentration of which was about 2.5 M. To about 6.7 mL of this solution 1.11 mL of the trisodium citrate solution were added to produce the pH 5 solution in which the concentration of LDEE.HCl was about 2.1 M. After flushing with nitrogen, each solution was kept in a vial sealed with a grey, oxygen impermeable septum.

The observed percentages of LDEE.HCl hydrolyzed per hour at 4° C., 37° C. and 55° C. are summarized in Table 3. For example, at pH 2.3 and 55° C., 1.9% of the LDEE.HCl was hydrolyzed per hour.

TABLE 3

| pH | 4° C. | 37° C. | 55° C. |
|---|---|---|---|
| 2.3 | 0.0000 | 0.004 | 0.019 |
| 3.0 | 0.0001 | 0.003 | 0.02 |
| 4.0 | 0.0005 | 0.014 | 0.027 |
| 5.0 | 0.0029 | | |

### Example 18

### Shelf Life of 2.5 M LDEE.HCl at pH 2.3 and at 4° C.

An aqueous LDEE.HCl solution was prepared by dissolving 6.002 g of LDEE in 5.2 mL of 5.0M HCl. An about 1 M trisodium citrate solution was prepared of 1.353 g of trisodium citrate dihydrate and 4.0 g of water. Of this 1 M trisodium citrate solution 0.3 mL were added to the LDEE.HCl solution. The volume was about 10.6 mL and the LDEE.HCl concentration was about 2.5 M. The pH of the solution was 2.3. After flushing with nitrogen, each solution was kept in a vial sealed with a grey, oxygen impermeable septum. The solution was kept in a refrigerator at about 4° C. and its LD content was determined initially daily then weekly for 21 weeks. In the 21 week refrigerated storage period the LD concentration increased from 0.17% of that of the LDEE.HCl (i.e., from 0.0043 M) to 0.47% of that of the LDEE.HCl (i.e., to 0.0117 M), a change of 0.0074 M. With the solubility of LD being (per Example 16) about 17 g/L or 0.086 M, saturation in LD is expected after about 244 weeks or about 4.7 years of storage at 4° C. The projected shelf life of the 2.5 M solution at about 4° C. is about 4.7 years. In this period the solution is expected to remain free of LD precipitate.

### Example 19

### Shelf Life of 2.7 M LDEE.HCl at pH 2.3 and at 22.6±1.0° C.

An about 1 M trisodium citrate solution was prepared by dissolving in 1.353 g of trisodium citrate dihydrate in 4.0 mL water. 1.501 g (6.67 millimoles) of LDEE were dissolved in 1.11 mL of aqueous 6 M HCl to form a pH 0.3 solution, the pH of which was increased to 2.3 by adding 0.055 mL of the 1 M trisodium citrate solution. The volume was about 2.4 and the LDEE.HCl concentration was about 2.7 M. After flushing with nitrogen, each solution was kept in a vial sealed with an elastomeric grey, oxygen impermeable septum. There was a measurable, but small, increase in LD concentration in the 9 day period of storage at the ambient temperature of 22.6±1.0° C. as is shown in Table 4. In the 8 day period following Day 1, about 0.026% of the LDEE.HCl was hydrolyzed daily to LD.

TABLE 4

| | Elapsed time (days) | | | | | | |
|---|---|---|---|---|---|---|---|
| | 1 | 2 | 5 | 6 | 7 | 8 | 9 |
| % of LDEE•HCl | 0.18 | 0.18 | 0.27 | 0.32 | 0.32 | 0.36 | 0.38 |
| hydrolyzed to LD | | | | | | | |

LD precipitation may occur from the 2.7 M LDEE.HCl solution at the saturation point of LD, which is according to Example 16 is greater than about 17 grams per liter at about 23±2° C. and at a pH of 4.5. Such a concentration is reached when about 3.4%, i.e., an additional 3.2% of the LDEE.HCl is hydrolyzed. Considering that about 0.024% of the LDEE.HCl is hydrolyzed daily, the projected shelf life at ambient temperature is about 128 days.

### Example 20

### Shelf Life of 2.5 M LDEE.HCl at pH 3.0 at 4° C.

An aqueous LDEE.HCl solution was prepared of 6.002 g of LDEE and 5.2 mL of 5.0 M HCl. An about 1 M trisodium citrate solution was prepared of 1.353 g of trisodium citrate dihydrate and 4.0 g of water. 300 μL of the 1 M trisodium citrate were added to the LDEE.HCl solution. The volume was about 10.6 mL and the LDEE.HCl concentration was about 2.5 M. To 9.1 mL of this solution 80 μL of the 1M citrate solution were added to produce about 9.2 mL of the about 2.5 M pH 3.0 solution. The solution was distributed in three vials. After flushing with nitrogen, each solution was kept in a vial sealed with a grey, oxygen impermeable septum. The solution was kept in a refrigerator at about 4° C. and the percentage of its LD hydrolyzed was about weekly for 21 weeks following its preparation. The results are tabulated in Table 5.

TABLE 5

| | Elapsed Time (weeks) | | | | | | |
|---|---|---|---|---|---|---|---|
| | 0 | 3 | 7 | 11 | 15 | 19 | 21 |
| % LDEE•HCl | 0.15 | 0.23 | 0.31 | 0.37 | 0.43 | 0.49 | 0.51 |
| hydrolyzed | | | | | | | |

LD precipitation from an LDEE.HCl solution may occur at the saturation point, which is according to Example 16 is about 17 grams per liter at about 23±2° C. and at pH 4.5. This concentration is reached in a 2.5 M LDEE.HCl solution when about 3.4% of the LDEE.HCl is hydrolyzed. In the 21 week period of the experiment 0.36% of the 2.5 M LDEE.HCl was hydrolyzed in addition of the initially present 0.15%. It is projected therefore that the solution should be free of LD precipitate for about 195 weeks or 3.8 years.

### Example 21

### Slow Hydrolysis of 2.4 M LDEE.HCl at pH 4.0 and at 4° C.

An aqueous LDEE.HCl solution was prepared of 6.002 g of LDEE and 5.2 mL of 5.0M HCl. An about 1 M trisodium citrate solution was prepared of 1.353 g of trisodium citrate dihydrate and 4.0 g of water. 300 μL of the 1 M trisodium citrate were added to the LDEE.HCl solution. The volume was about 10.6 mL and the LDEE.HCl concentration was about 2.5 M. To 9.1 mL of this solution 80 μL of the 1M citrate solution were added to produce about 9.2 mL of an about 2.5 M solution. To 7.7 mL of this solution 180 μL of the 1 M citrate solution were added. The volume of the resulting solution was about 7.9 mL and its pH was about 4.0. The LDEE.HCl concentration was about 2.4 M. The solution was distributed in three vials. After flushing with nitrogen, each solution was kept in a vial sealed with a grey, oxygen impermeable septum. The solution was kept in a refrigerator at about 4° C. and its LD content was tracked for 21 weeks. The results are tabulated in Table 6.

TABLE 6

| | Elapsed time (weeks) | | | | | |
|---|---|---|---|---|---|---|
| | 0 | 3 | 7 | 11 | 15 | 21 |
| % of LDEE•HCl hydrolyzed | 0.16 | 0.39 | 0.76 | 1.02 | 1.22 | 1.50 |
| to LD | | | | | | |

LD precipitation from an LDEE.HCl solution may occur at the saturation point, which is according to Example 16 is about 17 grams per liter at about 23±2° C. and at pH 4.5. This concentration is reached in an about 2.6 M LDEE.HCl solution when about 3.4% of the LDEE.HCl (3.2% above that at the start of the experiment) of is hydrolyzed. In the 21 week period of the experiment 1.34% of the 2.4 M LDEE.HCl was hydrolyzed in addition to the initially present 0.16%. It is projected therefore that the refrigerated pH 4.0 solution should be free of LD precipitate for about 50 weeks.

### Example 22

### Rapid Hydrolysis of 2.1 M LDEE.HCl at 4° C. And at pH 5.0

An aqueous LDEE.HCl solution was prepared of 6.002 g of LDEE and 5.2 mL of 5.0M HCl. An about 1 M trisodium citrate solution was prepared of 1.353 g of trisodium citrate dihydrate and 4.0 g of water. 300 μL of the 1 M trisodium citrate were added to the LDEE.HCl solution. The volume was about 10.6 mL and the LDEE.HCl concentration was about 2.5 M. To 9.1 mL of this solution 80 μL of the 1M citrate solution were added to produce about 9.2 mL of an about 2.5M solution. To 7.7 mL of this solution 180 μL of the 1 M citrate solution were added. The volume of the resulting solution was about 7.9 mL and its LDEE.HCl concentration was about 2.4 M. To 6.4 mL of this solution 1.14 mL of the 1M citrate solution were added to produce an about 2.1 M LDEE.HCl solution of pH 5. The solution was distributed in three vials. After flushing with nitrogen, each solution was kept in a vial sealed with a grey, oxygen impermeable septum. The solution was kept in a refrigerator at about 4° C. and its LD content was tracked for 15 days. The results are tabulated in Table 7.

TABLE 7

| | Elapsed time, days | | | | |
|---|---|---|---|---|---|
| | 1 | 4 | 8 | 12 | 15 |
| % of LDEE•HCl hydrolyzed to LD | 0.19 | 0.72 | 0.97 | 1.24 | 1.42 |

As seen in Table 7, as the hydrolysis progressed the rate of hydrolysis slowed. The slowing of the rate of hydrolysis is attributed to the decrease in pH upon substantial hydrolysis. This decrease results of the increase in carboxylic acid functions as the LDEE.HCl hydrolyzes to LD.HCl and ethanol.

### Example 23

### Longer than 48 h Operational Life at Body Temperature of an about pH 4.5 Solution Containing the Molar Equivalent of about 0.532 g L-DOPA Per mL

A 2.9 M LDEE.HCl solution of about pH 2.41 was prepared. It was formed by mixing at 0-4° C. (a) 20 weight % HCl; (b) LDEE and (c) trisodium citrate dihydrate at the ratio 44.9559 weight %:54.3967 weight %:0.0065 weight %. 2.5 mL or about 3.0 g of this solution were transferred to each vial. To a second vial 0.25 mL of 1.5 M trisodium citrate was added. 0.20 mL of the trisodium citrate solution in the second vial were then transferred with a syringe to the 2.9 M LDEE.HCl solution containing vial. Upon the transfer, an infusible, about 2.7 M, about pH 4.49 LDEE.HCl solution, containing the molar equivalent of about 0.532 g L-DOPA per mL was obtained. The typical daily dose of 1.0 g L-DOPA can be delivered by infusion of about 1.88 mL of this solution. Because the solution may be near body temperature when worn in a skin-contacting container, its operational life at 40° C. was determined. Initially, the LD:LDEE molar ratio was 0.1:99.9. After 24 h at 40° C. the LD:LDEE molar ratio increased to 1.8:98.2 and after 48 h to 2.8:97.2. Because of the hydrolysis, the pH dropped after 24 h to 4.2 and after 48 h to 4.0. Because LD does not precipitate from 2.7 M LDEE.HCl at a pH between 4.0 and 4.6 until the LD:LDEE molar ratio exceeds 3.4:96.6, the operational life at 40° C. suffices for more than the targeted 16 h, 24 h and 48 h long infusions.

### Example 24

### Operational Stability of Subcutaneously Infusible Solutions

To determine whether hydrolysis of subcutaneously infusible solutions of 0.95 M and 0.48 M LDEE.HCl concentrations excessively lowers their pH or drives their LD concentration beyond the saturation point where LD could precipitate, their starting pH of less <3 adjusted by adding an about 1.5 M trisodium citrate solution, were kept at 40° C. for 24 hrs. While their pH did drop, the decrease was small, and the LD concentration remained below the estimated saturation concentration up to about pH 5.5. The results are shown in Tables 8 and 9.

TABLE 8

| pH of about 0.95M | | | | pH of about 0.48M | | | |
|---|---|---|---|---|---|---|---|
| LDEE•HCl Solution | | | | LDEE•HCl Solution | | | |
| 0 h | 2 h | 6 h | 24 h | 0 h | 2 h | 6 h | 24 h |
| 4.2 | 4.19 | 4.02 | 3.86 | 4.19 | 4.43 | 4.34 | |
| 4.4 | 4.38 | 4.28 | 4.03 | 4.45 | 4.58 | 4.54 | 4.19 |
| 4.6 | 4.61 | 4.53 | 4.22 | 4.59 | 4.68 | 4.6 | 4.32 |
| 4.82 | 4.74 | 4.69 | 4.36 | 4.78 | 4.85 | 4.71 | 4.52 |
| 5.01 | 5.01 | 4.84 | 4.58 | 5.01 | 5.01 | 4.9 | 4.6 |
| 5.3 | 5.34 | 5.21 | 4.8 | 5.31 | 5.42 | 5.23 | 4.91 |
| 5.6 | 5.68 | 5.53 | 5.19 | 5.61 | 5.65 | 5.53 | 5.23 |

In the same experiment, also the time dependence of the LD/LDEE HPLC peak ratios was measured. The results are shown in Table 9. Division of the ratios by 0.94 provides the actual LD/LDEE molar ratio.

TABLE 9

| Elapsed | Initial | 0.95M | 0.48M |
|---|---|---|---|
| time | pH | LDEE•HCl | LDEE•HCl |
| 0 hrs | 4.2 | 0.1238 | 0.1292 |
| | 4.4 | 0.1252 | 0.1309 |
| | 4.6 | 0.128 | 0.133 |
| | 4.8 | 0.1354 | 0.1352 |
| | 5 | 0.1467 | 0.1418 |
| | 5.3 | 0.1628 | 0.1547 |
| | 5.6 | 0.1909 | 0.1902 |
| 2 hrs | 4.2 | 0.2073 | 0.2171 |
| | 4.4 | 0.2404 | 0.2636 |
| | 4.6 | 0.2994 | 0.2998 |
| | 4.8 | 0.3995 | 0.3758 |
| | 5 | 0.5443 | 0.5202 |
| | 5.3 | 0.8361 | 0.8609 |
| | 5.6 | 1.3007 | 1.4028 |
| 6 hrs | 4.2 | 0.2872 | 0.3079 |
| | 4.4 | 0.3618 | 0.3994 |
| | 4.6 | 0.4867 | 0.4804 |
| | 4.8 | 0.7068 | 0.6546 |
| | 5 | 1.0212 | 0.9805 |
| | 5.3 | 1.6832 | 1.7239 |
| | 5.6 | 2.7417 | 2.8727 |
| 24 hrs | 4.2 | 0.7792 | 0.8294 |
| | 4.4 | 1.0438 | 1.1405 |
| | 4.6 | 1.4684 | 1.415 |
| | 4.8 | 2.2128 | 1.9804 |
| | 5 | 3.2875 | 3.0727 |
| | 5.3 | 5.6263 | 5.6312 |
| | 5.6 | 9.5612 | 9.6563 |

### Example 25

### Subcutaneous Infusion in Minipigs

Citrate buffered LDEE.HCl solutions having a pH of 4.5-5.0 were infused into the shoulders of juvenile minipigs weighing about 10 kgs. The results are provided in Table 10.

TABLE 10

| | | | Total | | Symptoms |
|---|---|---|---|---|---|
| Mini | Exper. | Conc., | Dose, | Infusion, | at end of |
| Pig | # | mg/mL | mg | hrs | infusion |
| A | 1 | 608 | 163 | 16 | None |
| B | 1 | 608 | 163 | 16 | small hard |
| | | | | | mass |
| A | 2 | 214 | 489 | 16 | none |
| B | 2 | 214 | 489 | 16 | none |
| A | 3 | 214 | 1142 | 16 | diffuse soft |
| | | | | | mass, 2 × 4 cm |
| B | 3 | 214 | 1142 | 16 | diffuse soft |
| | | | | | mass, 2 × 4 cm |
| A | 4 | 108 | 1142 | 16.5 | none |
| B | 4 | 108 | 1142 | 16.5 | none |

The concentrations and doses listed in Table 10 are LDEE (MW 225) based.

### Example 26

### High Rate Intramuscular Infusion of 2.3 M LDEE.HCl of pH 4.5 in Human

About 0.45 mL of the 2.3 M LDEE.HCl citrate buffered to pH 4.5 of Example 23 were intramuscularly infused over a 45 min period in a 78 year old male volunteer using a Medronic Minimed Paradigm 522 insulin pump with the Quickset Paradigm 23 inch (60 cm) infusion set. The cannula was inserted intramuscularly in the right upper arm's side facing away from the chest at about 5 mm depth. The infused solution was colorless and clear (meaning precipitate-free) after being kept for about 3 days at room temperature and for about 3 months in a refrigerator. The infused solution contained about 1 millimoles of LDEE.HCl, the equivalent of about 0.2 g LD. The flow rate was 0.6 mL/h. After the infusion there was a small nodule which spread after 4 hours into an about 1 mm high, 2-3 cm radius protrusion. There was no pain, inflammation, color change or sensitivity at the protruding site. The protrusion disappeared after 2 days.

### Example 27

### Painless Subcutaneous Infusion 2.3 M LDEE.HCl of pH 4.5 in Human

0.24 mL of the 2.3 M LDEE.HCl citrate buffered to pH 4.5 of Example 23 was subcutaneously infused in a 78 year old male volunteer over an about 90 min period in 8 boluses spaced 12 min apart. Each bolus was about 3 min long; during the 8 infusion periods of 3 min the flow rate was about 10 μL/min. A Medronic Paradigm insulin pump with the Quickset Paradigm 23 inch (60 cm) infusion set was used for infusion. The set had an about 1 cm long needle and cannula, which was used to insert the cannula. The cannula was inserted with its tip about 9 mm deep vertically just left of the center of the abdomen, about 5 cm below the diaphragm, probably in the abdominal fat. The infused solution was colorless and clear (meaning precipitate-free) after being kept for 2.5 days at room temperature and for 7 weeks in a refrigerator. Even though the pH was mildly acidic and the solution was hypertonic, the volunteer felt no pain or irritation during or after the infusion, and the infusion did not change the appearance of the skin, nor did it cause the formation of a nodule. The 240 μL infused over the 90 min period contained the equivalent of about 110 mg of LD, meaning that the dose rate was equivalent of 73 mg/hr LD.

### Example 28

### Abdominal Subcutaneous Infusion of 2.7 M LDEE.HCl in Human

A healthy 49 year old male volunteer took one Lodosyn (carbidopa), 25 mg pill, at each of the morning, midday and evening of the day preceding the infusion, and in the morning, midday and evening of the day of the infusion. After adjusting the pH of a stored solution of pH 2.4 and 2.5 mL volume of 2.9 M LDEE.HCl by adding to it 0.16 mL of 1.5 M trisodium citrate solution to about pH 4.6±0.1, a 2.7 M LDEE.HCl solution was obtained. Using a Medtronic Minimed Paradigm 723, a Medtronic Minimed Silhouette, 43 inches, 17 mm cannula infusion set, a Medtronic Minimed Sil-Serter infusion set insertion system, the volunteer infused 0.67 mL of the 2.7 M LDEE.HCl solution into his abdomen over a period of 9 hours. The infusion site was 7-8 cm below the ribs. The initial 4 hr infusion was at a rate of 0.062 mL/hr (37.5 mg of LDEE/hr), followed by 0.082 mL/hr (50 mg of LDEE/hr) in the next 5 hours. The total infused volume contained 406 mg LDEE, the equivalent to 356 mg L-DOPA. There was virtually no pain or redness during or after the infusion. Mild swelling was observed and an induration of approximately 2.5 cm diameter developed under the infusion site. After 24 hours the induration became diffuse and partly, but not completely, subsided. There were no other adverse events.

### Example 29

### Subcutaneous Infusion of 2.7 M LDEE.HCl in the Exercised Upper Arm of a Volunteer

A healthy 49 year old male volunteer took one Lodosyn (carbidopa), 25 mg pill, at each of the evening preceding the infusion, and in the morning, midday and evening of the day of the infusion. After adjusting the pH to about pH 4.7±0.3 of a stored solution of pH 2.4 and 2.5 mL volume of 2.9 M LDEE.HCl by adding 0.16 mL of 1.5 M trisodium citrate solution, a 2.7 M LDEE.HCl solution was obtained. Using a Medtronic Minimed Paradigm 723, a Medtronic Minimed Silhouette, 43 inches, 17 mm cannula infusion set, and a Medtronic Minimed Sil-Serter infusion set insertion system, the a volunteer infused 0.67 mL of the 2.7 M LDEE.HCl solution into the frequently exercised and occasionally massaged outside of the upper arm over a period of 9 hours. The initial 4 hr infusion was at a rate of 0.062 mL/hr (37.5 mg of LDEE/hr), followed by 0.082 mL/hr (50 mg of LDEE/hr) in the next 5 hours. The total infused volume contained 400 mg LDEE, the equivalent to 351 mg L-DOPA. There was virtually no pain or redness during or after the infusion, nor was there an obvious induration, but an about 7 cm long elongated mildly swollen region was observed at the infusion site at the end of the infusion. The swelling dissipated gradually and disappeared after a week. There were no other adverse events of any kind.

### Example 30

### Subcutaneous Infusion of 0.95 M LDEE.HCl in the Exercised Upper Arm of a Volunteer

A healthy 49 year old male volunteer took one Lodosyn (carbidopa), 25 mg pill, at each of the evening of the day preceding the infusion, and in the morning, mid-day and evening of the day of the infusion. 1 mL of the 2.9 M LDEE.HCl stored solution of pH 2.4, 0.04 mL of a 1.5 M trisodium citrate solution and 2 mL sterile water were syringed into the reservoir of a Medtronic Minimed Paradigm 723 pump. This pump and a Medtronic Minimed Silhouette, 43 inches, 17 mm cannula infusion set inserted with a Medtronic Minimed Sil-Serter infusion set insertion system were used. The volunteer infused 1.85 mL, or 396 mg LDEE (equivalent to 347 mg L-DOPA) of the now 3× diluted 0.95 M LDEE solution into his upper arm over a period of 9 hours at two rates: First near 37.5 mg/hr (0.175 mL/hr) for 4 hours then near 50 mg/hr (0.233 mL/hr) for 5 hours. During the course of the infusion the volunteer periodically exercised his arm and gently massaged the infusion site. There was no pain or redness during or inflammation after the infusion. At the infusion site there was a very slightly swollen region of about 4 cm diameter at the end of the infusion, which disappeared gradually in about three days. There were no other adverse events.

### Example 31

### Subcutaneous Infusion of 0.58 M LDEE.HCl in the Upper Arm of a Volunteer

A healthy 78 year old male volunteer took one Lodosyn (carbidopa), 25 mg pill, at each of the evening of the day preceding the infusion, and in the morning, mid-day and evening of the day of the infusion. The reservoir of a Medtronic Minimed Paradigm 723 pump was primed (to assure that all of the 3 mL in the reservoir will be infused) then the reservoir was filled with 0.6 mL of the 2.9 M LDEE.HCl stored solution of pH 2.4, 0.024 mL of a 1.5 M trisodium citrate solution and 2.4 mL sterile water by syringing. The pump, a Medtronic Minimed Silhouette, 43 inches, 17 mm cannula infusion set, and a Medtronic Minimed Sil-Serter infusion set insertion system were used. The volunteer infused 3 mL, or 400 mg LDEE (equivalent to 350 mg L-DOPA) of the now 5× diluted (i.e. 0.58 M) LDEE solution into his upper arm over a period of 9 hours at a flow rate of 0.33 mL/hr. The volunteers felt no pain during the infusion. At the end of the infusion the volunteer had no pain and only very slight reddening of a 2×4 cm skin area and a very slightly harder than normal 2 cm diameter zone under the skin at the infusion site. The infusion ended at 7:30 pm. On the following morning at 5:30 am, the skin and infused tissue appeared normal, i.e., they could not be distinguished from those of the contra-lateral, non-infused arm. There were no adverse events.

### Example 32

### Subcutaneous Infusion of 5.1 Millimoles of 0.48 M LDEE.HCl in the Upper Arm

A healthy 78 year old male volunteer took 1 Lodosyn (carbidopa), 25 mg pill, at each of the early afternoon and late evening of the day preceding the infusion, and in the morning, mid-day and evening of the day of the infusion. The reservoir of a Medtronic Minimed Paradigm 723 pump was primed, the reservoir was filled then refilled three times over the course of the infusion with a total of 10.7 mL of the infused 0.48 M LDEE.HCl, pH 4.8-5.0 citrate buffered solution. The pump, a Medtronic Minimed Silhouette, 43 inches, 17 mm cannula infusion set, and a Medtronic Minimed Sil-Serter infusion set insertion system were used. The volunteer infused 1,154 mg LDEE (5.1 millimoles, equivalent to 1,011 mg L-DOPA) of the 0.48 M LDEE.HCl solution into his upper arm over a period of 18 hours at an average flow rate of about 0.6 mL/hr. The volunteer felt no pain during the infusion. At the end of the infusion the volunteer had no pain, reddening or inflammation but observed swelling most of which subsided within 8 hours after the end of the infusion and was no longer visible after 12 hrs. There were no adverse events.

### Example 33

### Long Shelf Life Two Vial System, One with Solid LDEE, the Other with HCl

A first glass vial would contain 1.5 g (6.67 millimoles) of dry LDEE and 20 mg (0.0.78 millimoles) of anhydrous trisodium citrate. A second vial would contain aqueous 0.5 M HCl (13.4 mL, 06.67 millimoles). The expected LDEE.HCl concentration in the solution resulting of mixing the contents of the vials would be about 0.5M and the pH of the solution would be about 4.7±0.5. A shelf life of at least 1 year at 25° C. is expected for the unmixed constituents of the vials. After mixing the operational life is expected to exceed 24 hrs at 37° C.

### Example 34

### Exemplary Two-Vial Embodiment for Subcutaneous Infusion

The user is to be provided with two vials. Vial A contains 3 mL of the 2.5 M LDEE.HCl aqueous solution. The LDEE.HCl solution is acidic (has an about 0.45 millimole excess of HCl). Enough 1 M trisodium citrate is added to increase the pH to about 2.5±0.5. The solution can be stored when refrigerated, according to Example 18, for several years. The 7.5 millimoles of LDEE.HCl contained in Vial A are the equivalent of 1.5 g of LD. Vial B contains a trisodium citrate solution of about 0.1 M concentration in the amount needed for raising the pH in Vial A from about pH 2.5±0.5 to about 4.5, typically about 10 micromoles or about 0.1 mL. Prior to infusion the solutions stored in the two vials are made operational by their mixing.

### Example 35

### Exemplary Single-Vial Embodiment for Subcutaneous Infusion

The vial may contain 10 mL of a 0.5 M LDEE.HCl pH 4 citrate buffered aqueous solution. The 5 millimoles of LDEE.HCl contained in the vial are the equivalent of about 1 g of LD. For storage, the vial would be refrigerated at 5±3° C. The user or caregiver would be instructed to infuse the solution, as is, within about 6 months of its production and refrigerated storage.

### Example 36

### 2.9 M LDEE.HCl of pH 2.5±0.5 for Intragastric, Intraduodenal or Intrajejunal Infusion

For intragastric, intraduodenal or intrajejunal infusion, a vial may contain 3.7 mL of 2.9 M LDEE.HCl, its pH adjusted by monosodium citrate and HCl to 3.0±0.2. The vial would contain about 10.7 millimoles of LDEE.HCl, enough for 2 days for a patient requiring about 1 g of LD per day. The vial may be stored refrigerated for at least 12 month and additionally kept at the body temperature of about 37° C. for at least 2 days. Its content may be infused as is, without raising the pH, using a tubing of less than 1 mm internal diameter and less than 2 mm outer diameter.

### Example 37

### Water Free LDEE Linoleate Salt for Intragastric, Intraduodenal or Intrajejunal Infusion

To 28 g (MW 280, 0.02 moles) of linoleic acid maintained at 20° C. in a water bath and stirred under a dry nitrogen atmosphere, a solution of 19.5 g of LDEE (0.0173 moles) in 50 mL dry de-aerated methanol was added drop-wise at a rate slow enough to prevent the temperature from rising above 25° C. After completion of the addition, the methanol was flash evaporated in vacuo (at about 1 mm pressure) at 30° C. The resulting oil was collected and stored in a refrigerator in vials under dry nitrogen, each vial containing about 3 g of the colorless viscous liquid. The concentration of LDEE linoleate is about 2 M, i.e. 2.5 mL contain the pharmaceutical equivalent of 1 g LD. The viscous solution could be stored refrigerated at 5±3° C. for at least 3 months. It could be intragastrically or intrajejunally infused for managing PD. To reduce its viscosity it could be diluted with an edible oil or with an edible liquid carboxylic acid, such as linoleic acid or oleic acid.

### Example 38

### LDE Solid Formulation

A solid formulation of the invention can include the components tabulated in Table 11 below.

TABLE 11

| | Compound | Description |
|---|---|---|
| Levodopa ester | LDEE | |
| Acid | Hyaluronic acid | Its number of carboxylic |
| | | acid functions exceeding |
| | | the number of LDEE |
| | | molecules |
| Buffer | Sodium hyaluronate | |
| Anti-oxidant | Acetaminophen, | Less than 5 wt % |
| | p-aminophenol, or | |
| | t-butyl | |
| | ortho-substituted phenol | |
| Crystal growth | Hyaluronic acid/salt | As above |
| inhibitor | | |
| Additional PD | Carbidopa | |
| therapeutic | | |
| Anti-pain/anti- | Propofol, ibuprofen, | Less than 5 wt % |
| inflammatory | lidocaine | |
| Container | Aluminized high density | |
| | polypropylene or polyester. | |

### Example 39

### Non-Aqueous Liquid Salt of LDEE

A non-aqueous liquid salt of LDEE for subcutaneous or intragastric or intrajejunal infusion can include a liquid or liquid crystalline ricinoleic acid, or oleic acid, or palmitic acid addition salt of L-DOPA ethyl ester (0-10 mole % excess acid), and may also include

(i) Vitamin E, 1.5 weight % (antioxidant).

(ii) Lidocaine, 0.1 weight % (analgesic).

The components are placed in a 5 mL container and stored under nitrogen and refrigerated to retard precipitation of the addition salt.

### Example 40

### Non-Aqueous Glycerol Solution of LDEE

A non-aqueous glycerol solution of LDEE for subcutaneous or intragastric or intrajejunal infusion can include 25 weight % LDEE, and some or all of the components below.

(i) Vitamin E, 0.5 weight % (antioxidant),

(ii) Lidocaine, 0.1 weight % (analgesic), and

(iii) Glycerol.

The components are placed in a 10 mL container and stored at ambient temperature under nitrogen.

### Example 41

### Co-Infusion of Concentrated Aqueous Solutions Containing Prodrugs of Carbidopa and LD

A vial containing 2.5 mL of an aqueous solution having a pH of 2.5±0.5 and containing the hydrochloride salts of ethyl esters of both LDEE and carbidopa at respective molar concentrations of 2.4±0.6 M and 0.6±0.2 M could be stored refrigerated at 5±3° C. for at least 6 months. It would be infused as is intraduodenally or intrajejunally or intragastrically. Prior to subcutaneous infusion its pH could be raised to between 4 and 6 by adding 0.3±0.03 millimoles of trisodium dissolved in 12.5 mL water.

### Example 42

### Pharmacokinetic Study of Infusion of LDEE.HCl Formulation in Minipigs

Citrate buffered LDEE.HCl solutions having a pH of 4.5-5.0 were infused into the neck/shoulder regions of juvenile minipigs weighing about 10 kgs. Two minipigs were treated. On each minipig, the solutions were simultaneously infused at two infusion sites, one on the left neck/shoulder and one on the right neck/shoulder. A total of 5.24 mL of solution containing 218 mg/mL LDEE was infused into each minipig over a period of 16 hours, delivering a total dose of 1,142 mg LDEE to each minipig. The dose was split equally between the two infusion sites, with each infusion site receiving 571 mg LDEE in 2.62 mL over 16 hours.

Blood samples were collected at times 0 (pre-dose), 30 minutes, and 1, 2, 4, 8 and 16 hours after the start of the infusion. Approximately 3 mL whole blood was collected from the jugular vein. The whole blood was collected into tubes containing K2 EDTA as the anticoagulant. Immediately after collection the samples were centrifuged for ˜15 minutes at ˜3000 RPM, at ˜4° C. Each plasma sample was then transferred to a plastic cryovial containing sodium metabisulfite and sodium fluoride as preservatives. After the samples were processed they were placed on dry ice until movement to storage at ˜−70 degrees Celsius. The samples were analyzed for LDEE using LC-MS.

The minipigs experienced mild swelling in an area of about 1×2 cm at the infusion sites at the end of the infusion that resolved over 24 hours, and subsequent development of small subcutaneous nodules of about 1×1 cm that resolved over a period of about one week. The measured concentrations of LDEE and LD in the two minipigs are shown in Table 12.

TABLE 12

| | | LDEE Concentration | | LD Concentration | |
|---|---|---|---|---|---|
| | | (ng/mL) | | (ng/mL) | |
| Sampling Time | | Minipig #1 | Minipig #2 | Minipig #1 | Minipig #2 |
| Pre-dose | | 3.6 | 3.3 | 9.0 | 31.0 |
| 30 | minutes | 6.2 | 6.2 | 716.2 | 1180.7 |
| 1 | hour | 6.4 | 4.8 | 693.6 | 1052.2 |
| 2 | hour | 6.3 | 6.3 | 1535.2 | 1371.9 |
| 4 | hour | 6.8 | 7.9 | 1585.2 | 2489.7 |
| 8 | hour | 10.0 | 10.9 | 3184.9 | 2374.2 |
| 16 | hour | 10.0 | 11.5 | 1549.8 | 1830.9 |

In this study the minipigs were administered a dose of 1,142 mg LDEE over 16 hours, equivalent to the administration of 1.0 g of LD. This is the size of a typical LD oral dose administered to a patient with advanced PD. This dosing equates to 114.2 mg/kg for the 10 kg minipigs, versus 16.3 mg/kg for a typical 70 kg patient. The dose received by the minipigs is therefore a factor of seven higher, on a mg/kg basis, than a typical dose administered to patients with advanced PD.

The data demonstrate that even when the LDEE formulation was administered at a high dose of 114.2 mg/kg, the circulating plasma LDEE concentration did not exceed 15 ng/mL. It may be expected that at the lower doses (on a mg/kg basis) typically administered to PD patients, the circulating plasma LDEE concentration during the infusion will not exceed 100, 50, 30, 15, 10 or 5 ng/mL.

The data demonstrate that a circulating plasma LD concentration greater than 1,600 ng/mL was continuously maintained for a period of at least 8 hours during the infusion. This demonstrates that a circulating plasma LD concentration exceeding 400, 800, 1200, or 1600 ng/mL can be continuously maintained for a period of at least 8 hours during the infusion. The plasma LD concentrations achieved is sufficient to turn on a patient.

The data demonstrate that a circulating plasma LD concentration less than 5,000 ng/mL was continuously maintained for a period of at least 8 hours during said infusion. It may be expected that at the lower doses (on a mg/kg basis) typically administered to PD patients, the circulating plasma LD concentration during the infusion will not exceed 5000, 2500, or 2000 ng/mL.

The data demonstrate that a circulating plasma LD concentration greater than 700 ng/mL was achieved within 30 minutes of the initiation of the infusion, and that a circulating plasma LD concentration greater than 400 ng/mL was achieved within 60 minutes of the initiation of the infusion.

The circulating plasma LD concentrations obtained in this example are sufficient to turn a typical patient with advanced PD from the off to the on state.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

This application claims benefit of and priority to U.S. Provisional Application No. 61/421,902, filed Dec. 10, 2010, U.S. Provisional Application No. 61/431,256, filed Jan. 10, 2011, U.S. Provisional Application No. 61/492,227, filed Jun. 1, 2011, and U.S. Provisional Application No. 61/538,449, filed on Sep. 23, 2011, each of which is incorporated by reference herein in its entirety. Other embodiments are within the claims.

1-264. (canceled)

265. A method for treating treating Parkinson's disease in a subject, said method comprising subcutaneously infusing into the subject an LD prodrug solution comprising an LD prodrug, or a salt thereof, at such a rate that:
(a) the circulating plasma concentration of said LD prodrug during said infusion does not exceed 100 ng/mL; and
(b) a circulating plasma LD concentration greater than 400 ng/mL is continuously maintained for a period of at least 8 hours during said infusion.

266. The method of claim 265, wherein during said infusion the circulating LD plasma concentration varies by less than +/−20% from its mean for a period of at least 1 hour.

267. A pharmaceutical composition comprising an aqueous LD prodrug solution, wherein said LD prodrug solution comprises a sterile aqueous solution containing about 0.3 M to 4.0 M LDEE hydrochloride salt and having a pH of from 1.0 to 3.5.

268. A container comprising a material that is substantially impermeable to oxygen, said container containing a reconstitutable solid comprising an LD prodrug, or a salt thereof, wherein said container is substantially free of oxygen and wherein said reconstitutable solid, when reconstituted, is suitable for subcutaneous infusion.

269. A pharmaceutical composition comprising an aqueous LD prodrug solution containing greater than 0.3 M LD prodrug, or a salt thereof, wherein said pharmaceutical composition is substantially free of precipitated solid LD when stored at 5±3° C. for a period of 6 months, or at about 37° C. for a period of 24 hours, or when thawed after being stored frozen for at least 3 months.

270. The pharmaceutical composition of claim 269, wherein said pharmaceutical composition comprises a container, said container comprising a material that is substantially impermeable to oxygen and containing said aqueous liquid, wherein said container is substantially free of oxygen and wherein said aqueous liquid is suitable for subcutaneous infusion.

271. The pharmaceutical composition of claim 269, wherein said LD prodrug solution has a pH of from 4.0-6.0 or 4.0±0.5.

272. The pharmaceutical composition of claim 269, wherein said LD prodrug is LDEE, LDME, or a salt thereof.

273. The pharmaceutical composition of claim 272, wherein said LD prodrug is a hydrochloride salt.

274. The pharmaceutical composition of claim 269, wherein said LD prodrug solution comprises a buffer comprising citrate, succinate, pyrophosphate, or phosphate.

275. The pharmaceutical composition of claim 274, wherein said pharmaceutical composition is substantially free of oxygen.

276. The pharmaceutical composition of claim 269, wherein said pharmaceutical composition is supersaturated in LD.

277. The pharmaceutical composition of claim 269, wherein the solubility of LD in said LD prodrug solution is at least 5 g per liter at about 25° C.

278. A method for treating treating Parkinson's disease in a subject, said method comprising subcutaneously infusing into said subject a pharmaceutical composition of claim 269 in an amount sufficient to treat Parkinson's disease.

279. The method of claim 278, wherein said LD prodrug is subcutaneously infused at two or more infusion sites per day.

280. The method of claim 278, wherein said LD prodrug is subcutaneously infused intermittently at one or more infusion sites.

281. A method for treating treating Parkinson's disease in a subject, said method comprising subcutaneously infusing into said subject a pharmaceutical composition of claim 269 in an amount sufficient to treat Parkinson's disease, wherein said pharmaceutical composition is administered intragastrically, intraduodenally or intrajejunally.

282. An ambulatory infusion pump system for the treatment of Parkinson's disease comprising:
(i) a pharmaceutical composition of claim 269 in a drug reservoir; and
(ii) at least one cannula or needle in fluid communication with the drug reservoir for infusing said pharmaceutical composition into a subject.
